(19) United States
(12) Patent Application Publication
(10) Pub. No.: US 2017/0247756 A1
(43) Pub. Date: Aug. 31, 2017

(54) GENETIC SEQUENCE VERIFICATION COMPOSITIONS, METHODS AND KITS

(71) Applicant: Life Technologies Corporation, Carlsbad, CA (US)

(72) Inventors: Edgar Schreiber, Carlsbad, CA (US); Kamini Varma, Carlsbad, CA (US); Mark Andersen, Carlsbad, CA (US)

(21) Appl. No.: 15/515,758

(22) PCT Filed: Sep. 28, 2015
(86) PCT No.: PCT/US2015/052675

§ 371 Date: Mar. 30, 2017

Related U.S. Application Data
(60) Provisional application No. 62/059,821, filed on Oct. 3, 2014.
(60) Provisional application No. 62/059,824, filed on Oct. 3, 2014.

(51) Int. Cl. C12Q 1/68

(52) U.S. Cl. CPC C12Q 1/6874; C12Q 1/6806; C12Q 1/6853; C12Q 1/6858

(57) ABSTRACT

Methods, compositions and kits are described for resequencing, confirming or verifying Next Generation Sequencing (NGS) results with Sanger Sequencing. These methods are particularly useful for samples having very limited quantities such as formalin-fixed, paraffin-embedded (FFPE), Laser Capture Microdissection (LCM), fine needle biopsies or aspirates.

### CROSS REFERENCE

This application claims priority under 35 U.S.C. §119(e) to U.S. Provisional Application Nos. 62/059,821, filed Oct. 3, 2014, and 62/059,824, filed Oct. 3, 2014, the disclosures of which are hereby incorporated by reference in its entirety for all purposes.

### FIELD

Throughout this application various publications, patents, and/or patent applications are referenced. The disclosures of the publications, patents and/or patent applications in their entireties are hereby incorporated by reference into this application in order to more fully describe the state of the art to which this invention pertains.

The present teachings pertain to chemically modified oligonucleotide sequence primer compositions and methods for sequencing DNA and fragment analysis. The teachings also relate to compositions for preparing, fragment analysis and sequencing of nucleic acids such as cDNA and DNA. In particular, methods, compositions, systems, apparatuses and kits for amplifying one or more target sequences within a sample containing a plurality of target sequences are described. Optionally, a plurality of target sequences, for example at least 10, 50, 100, 500, or 1000, are amplified within a single amplification reaction. In some embodiments, the disclosure relates generally to methods, compositions, systems, apparatuses and kits for amplifying one or more target sequences from a single source, such as genomic DNA or formalin-fixed paraffin-embedded (FFPE) DNA. Methods of amplification and sequencing and compositions and kits thereof are described in this disclosure which can be used to verify the nucleotide sequence of the one or more target sequences from a single source by Sanger sequencing, in combination with/consecutively or after, sequencing by NGS methods.

### SUMMARY

In one aspect of the invention, a method for sequencing at least one amplicon is provided which includes the steps of: providing at least one amplicon, wherein the at least one amplicon comprises a sequence of interest and a preceding sequence 5′ to the sequence of interest incorporated from a first priming sequence; amplifying the at least one amplicon in a first reaction mixture which includes a plurality of nuclease-sensitive amplification primers to form an amplified DNA product; contacting the first reaction mixture containing the amplified DNA product with a second reaction mixture comprising a nuclease and at least one chemically-enhanced primer causing the plurality of nuclease sensitive amplification primers to be degraded by the nuclease; inactivating the nuclease; priming the amplified DNA product with the at least one chemically-enhanced primer in a sequencing reaction; and producing extension products of the at least one chemically enhanced primer. In some embodiments, the extension products may be fluorescently labeled. In various embodiments, the first priming sequence may have been used to produce the amplicon. In various embodiments, the first priming sequence may include at least one cleavable moiety. In some embodiments, the preceding sequence may be a portion of the first priming sequence. In various embodiments of the method, the steps of contacting the first reaction mixture with the second reaction mixture, inactivating the nuclease, and producing the extension products of the chemically enhanced primer may be performed in the same reaction vessel. In various embodiments, the steps of amplifying the at least one amplicon, contacting the first reaction mixture with the second reaction mixture, inactivating the nuclease, and producing the extension products of the chemically enhanced primer may be performed without intermediate purification steps.

In various embodiments of the method, the at least one amplicon further includes a succeeding sequence 3′ to the sequence of interest wherein the succeeding sequence is complementary to a second priming sequence used to produce the at least one amplicon. The at least one amplicon may have a length of about 100 nucleotides to about 400 nucleotides. The sequence of interest of the at least one amplicon may have a length of about 100 nucleotides to about 300 nucleotides. In other embodiments, the sequence of interest of the at least one amplicon may have a length of about 125 nucleotides to about 275 or about 250 nucleotides. The at least one amplicon may be a plurality of amplicons. In some embodiments, the plurality of amplicons may include at least two different amplicons, a first having a sequence of interest that is a major variant sequence and a second amplicon having a minor variant sequence from the same region of a sample nucleic acid.

In some embodiments, the method further includes the steps of obtaining sequencing results based on the sequencing reaction; and determining a nucleotide base sequence of at least the sequence of interest based on the results. The sequencing results may be obtained via a mobility based separation method. In some embodiments, the mobility based separation method may be capillary electrophoresis. In some embodiments, the determined nucleotide base sequence of at least the sequence of interest may be compared to a second nucleotide base sequence of at least the sequence of interest obtained from a NGS method of sequencing. The NGS method of sequencing may include massively parallel sequencing techniques like sequencing by synthesis using fluorophore or semiconductor detection and pyrosequencing, to name a few. In some embodiments, the NGS method of sequencing may be semiconductor sequencing.

In various embodiments, amplifying DNA may include polymerase chain reaction amplification. In selected embodiments, the sequencing reaction may include cycle sequencing.

In various embodiments, the first reaction mixture may also include a polymerase. The polymerase may be a thermostable polymerase. In some embodiments, the polymerase may be Taq polymerase. The first reaction mixture may further include deoxynucleotide triphosphates.

In various embodiments, the second reaction mixture further comprises a polymerase, deoxynucleotide triphosphates, and dye-labelled dideoxynucleotide triphosphates. The polymerase of the second reaction mixture may be a thermostable polymerase. In some embodiments, the polymerase is Taq polymerase.

In various embodiments of the method for sequencing at least one amplicon, the nuclease may be selected from exonuclease I, Exo III, Pfu and DNA pol I.

In various embodiments of the method for sequencing at least one amplicon,the chemically-enhanced primer may include an oligonucleotide sequence, a NCM and none or at least one nuclease-resistant linkage. In some embodiments, the chemically-enhanced primer may include one nuclease-resistant linkage at a terminal 3′ end. The chemically-enhanced primer may include a plurality of NCMs either at a terminal 5′ end or within a oligonucleotide sequence of the chemically-enhanced primer. In some embodiments the plurality of NCMs may be at a terminal 5′ end. In various embodiments, the NCM may be a (Cn) spacer wherein n is any integer from 1 to 9. The NCM may include a plurality of (Cn) spacers. In various embodiments, the chemically-enhanced primer may have a structure of the formula: (Cn)ₓ-OLIGO , where (Cn)ₓ has a structure of the following formula:

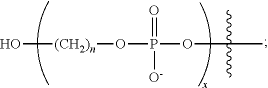

where each instance of n may independently be an integer of 1 to 9; and x may be an integer of 1 to about 30;
OLIGO has a structure of the following formula:

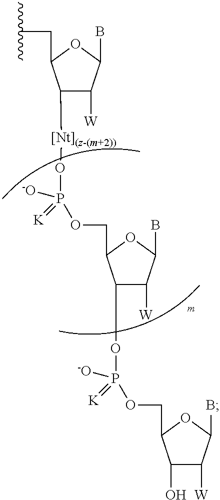

where B is a nucleobase; K is S or O; m is 0 or 1; z is an integer of 3 to about 100; W is OH, F, OMe, or H; and Nt is a moiety having a formula:

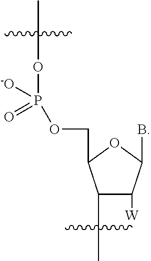

In some embodiments, the chemically enhanced primer may have any structure as described in this disclosure.

In various embodiments of the method for sequencing at least one amplicon, each of the plurality of nuclease- sensitive amplification primers may be configured to prime a sequence of interest of a specific disease state. In some embodiments, the plurality of nuclease-sensitive amplification primers may prime a set of sequences connected to a specific disease state.

In another aspect of the invention, a method is provided for confirming a DNA sequence, which includes the steps of: amplifying a sample comprising nucleic acid using at least a first priming sequence to provide a plurality of amplicons, where each of the plurality of amplicons includes a sequence of interest and a preceding sequence 5′ to the sequence of interest incorporated from a first priming sequence; amplifying a first aliquot of the plurality of amplicons in a first reaction mixture including a plurality of nuclease-sensitive amplification primers to form an amplified DNA product; contacting the first reaction mixture containing the amplified DNA product with a second reaction mixture that includes a nuclease and at least one chemically-enhanced primer, where by contacting the nuclease with the first reaction mixture, the nuclease sensitive amplification primers are degraded by the nuclease; inactivating the nuclease; priming the amplified DNA product with the at least one chemically-enhanced primer in a sequencing reaction; and producing extension products of the chemically enhanced primer. In some embodiments, the extension products may be fluorescently labeled. In various embodiments, the first priming sequence may have been used to produce the amplicon. The first priming sequence may include at least one cleavable moiety. In some embodiments, the preceding sequence may be a portion of the first priming sequence.

In various embodiments of the method, the steps of contacting the first reaction mixture with the second reaction mixture, inactivating the nuclease, and producing the extension products of the chemically enhanced primer may be performed in the same reaction vessel. In various embodiments, the steps of amplifying the plurality of amplicons, contacting the first reaction mixture with the second reaction mixture, inactivating the nuclease, and producing the extension products of the chemically enhanced primer may be performed without intermediate purification steps.

In various embodiments of the method, each of the plurality of amplicons further includes a succeeding sequence 3′ to the sequence of interest wherein the succeeding sequence is complementary to a second priming sequence used to produce the amplicon. Each of the plurality of amplicons may have a length of about 100 nucleotides to about 400 nucleotides. The sequence of interest of each of the plurality of amplicons may have a length of about 100 nucleotides to about 300 nucleotides. In other embodiments, the sequence of interest of each of the plurality of amplicons may have a length of about 125 nucleotides to about 275 or about 250 nucleotides. In some embodiments, the plurality of amplicons may include at least two different amplicons, a first having a sequence of interest that is a major variant sequence and a second amplicon having a minor variant sequence from the same region of a sample nucleic acid.

In some embodiments, the method further includes the steps of obtaining sequencing results based on the sequencing reaction; and determining a nucleotide base sequence of at least the sequence of interest based on the results. The sequencing results may be obtained via a mobility based separation method. In some embodiments, the mobility based separation method may be capillary electrophoresis. In some embodiments, the determined nucleotide base sequence of at least the sequence of interest may be compared to a second nucleotide base sequence of at least the sequence of interest obtained from a NGS method of sequencing performed on a second aliquot of the plurality of amplicons. The NGS method of sequencing may include massively parallel sequencing techniques like sequencing by synthesis using fluorophore or semiconductor detection and pyrosequencing, to name a few. In some embodiments, the NGS method of sequencing may be semiconductor sequencing.

In various embodiments, amplifying DNA may include polymerase chain reaction amplification. In selected embodiments, the sequencing reaction may include cycle sequencing.

In various embodiments, the first reaction mixture may also include a polymerase. The polymerase may be a thermostable polymerase. In some embodiments, the polymerase may be Taq polymerase. The first reaction mixture may further include deoxynucleotide triphosphates.

In various embodiments, the second reaction mixture further comprises a polymerase, deoxynucleotide triphosphates, and dye-labelled dideoxynucleotide triphosphates. The polymerase of the second reaction mixture may be a thermostable polymerase. In some embodiments, the polymerase is Taq polymerase.

In various embodiments of the method for confirming a DNA sequence, the nuclease may be selected from exonuclease I, Exo III, Pfu and DNA pol I.

In various embodiments of the method for confirming a DNA sequence, the chemically-enhanced primer may include an oligonucleotide sequence, a NCM and none or at least one nuclease-resistant linkage. In some embodiments, the chemically-enhanced primer may include one nuclease-resistant linkage at a terminal 3′ end. The chemically-enhanced primer may include a plurality of NCMs either at a terminal 5′ end or within a oligonucleotide sequence of the chemically-enhanced primer. In some embodiments the plurality of NCMs may be at a terminal 5′ end. In various embodiments, the NCM may be a (Cn) spacer wherein n is any integer from 1 to 9. The NCM may include a plurality of (Cn) spacers. In various embodiments, the chemically-enhanced primer may have a structure of the formula: (Cn)ₓ-OLIGO, where (Cn)ₓ has a structure of the following formula:

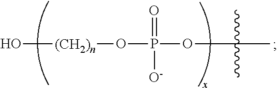

where each instance of n may independently be an integer of 1 to 9; and x may be an integer of 1 to about 30;
   OLIGO has a structure of the following formula:

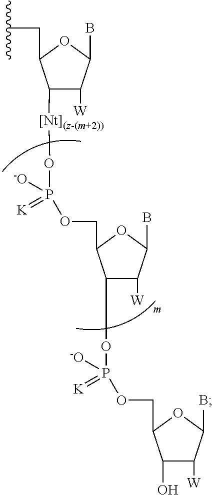

where B is a nucleobase; K is S or O; m is 0 or 1; z is an integer of 3 to about 100; W is OH, F, OMe, or H; and Nt is a moiety having a formula:

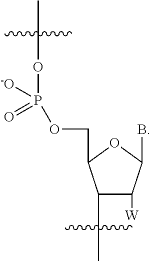

In some embodiments, the chemically enhanced primer may have any structure described in this disclosure.

In various embodiments of the method for confirming a DNA sequence, each of the plurality of nuclease-sensitive amplification primers may be configured to prime a sequence of interest of a specific disease state. In some embodiments, the plurality of nuclease-sensitive amplification primers may prime a set of sequences connected to a specific disease state.

In yet another aspect of the invention, a method is provided for preparing DNA for sequencing, including the steps of: amplifying a sample comprising nucleic acid using at least a first priming sequence to provide a plurality of amplicons, where each of the plurality of amplicons includes a sequence of interest and a preceding sequence 5′ to the sequence of interest incorporated from a first priming sequence; amplifying an aliquot of the plurality of amplicons in a first reaction mixture which includes nuclease-sensitive amplification primers to form an amplified DNA product; contacting the first reaction mixture containing the amplified DNA product with a second reaction mixture comprising a nuclease and a chemically-enhanced primer whereby the nuclease sensitive amplification primers are degraded by the nuclease; inactivating the nuclease; priming the amplified DNA product with the chemically-enhanced primer in a sequencing reaction; and producing extension products of the chemically enhanced primer.

In a further aspect of the invention, a method is provided for sequencing and verifying a variant nucleic acid sequence of interest, including the steps: amplifying a sample which includes nucleic acid using at least a first priming sequence to provide a plurality of amplicons, where each of the plurality of amplicons includes a sequence of interest and a preceding sequence 5′ to the sequence of interest incorporated from a first priming sequence; splitting the plurality of amplicons into a first aliquot and a second aliquot; amplifying the first aliquot of the plurality of amplicons in a first reaction mixture including nuclease-sensitive amplification primers to form a first amplified DNA product; contacting the first reaction mixture containing the first amplified DNA product with a second reaction mixture which includes a nuclease and a chemically-enhanced primer where by contacting the nuclease with the first reaction mixture, the nuclease sensitive amplification primers are degraded by the nuclease; inactivating the nuclease; priming the first amplified DNA product with the chemically-enhanced primer in a sequencing reaction; producing extension products of the chemically enhanced primer; obtaining sequencing results of at least the sequence of interest of the extended chemically enhanced primer using a mobility dependent separation; and determining a nucleotide base sequence of at least the sequence of interest of the extended chemically enhanced primer; amplifying the second aliquot of the amplicons to form a second DNA product; obtaining sequencing results of at least the sequence of interest of the second DNA product using a NGS sequencing method; and verifying a nucleotide sequence of the second DNA product by comparing it to the nucleotide base sequence of at least the sequence of interest of the extended chemically enhanced primer. In various embodiments of the method, the step of amplifying the second aliquot of the plurality of amplicons to form a second DNA product further comprises at least one of ligating adaptors, binding to beads, and ligating barcodes.

In another aspect of the invention, a composition for sequencing nucleic acid is provided that includes: a PCR amplification reaction product that comprises: a DNA product amplified from at least one amplicon, wherein the amplicon comprises a sequence of interest and a preceding sequence 5′ to the sequence of interest incorporated from a first priming sequence; non-nuclease-resistant amplification primer(s); and a chemically enhanced primer wherein the chemically enhanced primer comprises an oligonucleotide sequence, a NCM and none or at least one nuclease-resistant linkage.

The chemically-enhanced primer may include a plurality of NCMs either at a terminal 5′ end or within a oligonucleotide sequence of the chemically-enhanced primer. The NCM may be a (Cn) spacer wherein n can be any integer from 1 to 9. In various embodiments, the NCM comprises a plurality of (Cn) spacers. In various embodiments, the chemically-enhanced primer may have a structure of Formula I:

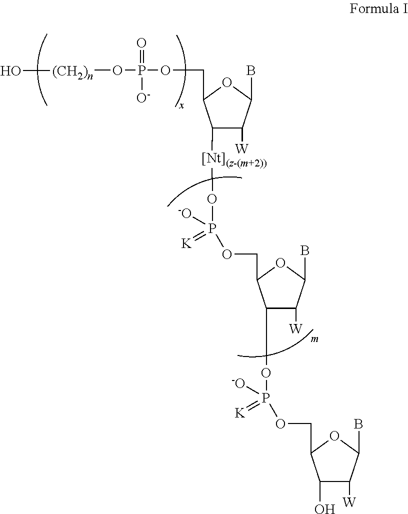

wherein B is a nucleobase; K is S or O; each n is independently an integer of 1 to 9; m is 0 or 1; × is an integer of 1 to about 30; z is an integer of 3 to about 100; W is OH, F, OMe, or H; and Nt is a moiety having a formula:

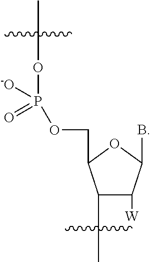

In some embodiments of the compositions of the invention, the chemically enhanced primer may be any chemically enhanced primer described in this disclosure.

In various embodiments, the oligonucleotide portion of the chemically-enhanced primer may include a universal primer. The universal primer may be selected from M13, US1, T7, SP6, and T3. The universal primer may be M13. In some embodiments, the chemically-enhanced primer may include one nuclease-resistant linkage.

In some embodiments of the compositions of the invention, the composition may further include a nuclease. In other embodiments, the composition may further include a polymerase, deoxynucleotide triphosphates, dideoxynucleotide triphosphates and a dye-label. In some embodiments, the dideoxynucleotide triphosphates may include dideoxynucleotide triphosphates labeled with the dye-label. The dye-labeled dideoxynucleotide triphosphates may include fluorescent dye-labeled dideoxynucleotide triphosphates. In some embodiments, the dye-label may be attached to the NCM or the oligonucleotide sequence.

In some embodiments of the compositions of the invention, the nuclease may be selected from exonuclease I, Exo III, Pfu and DNA pol I. In some embodiments, the polymerase may be Taq polymerase.

In some embodiments, the PCR amplification reaction product further includes an amplified DNA product where the DNA product is the amplification product of a plurality of amplicons.

In yet another aspect of the invention, a chemically enhanced primer is provided that includes an oligonucleotide sequence, at least one NCM and none or at least one nuclease-resistant linkage, and where at least 10 of the nucleotides at a 3′ terminus of the chemically enhanced primer are complementary to at least 10 of the nucleotides at the 5′ terminus of an amplicon, wherein the amplicon includes a sequence of interest and a preceding sequence 5′ to the sequence of interest incorporated from a first priming sequence.

In some embodiments, the chemically-enhanced primer comprises one nuclease-resistant linkage at the terminal 3′ end. The chemically-enhanced primer may include a plurality of NCMs either at a terminal 5′ end or within an oligonucleotide sequence of the chemically-enhanced primer. The NCM may be a (Cn) spacer wherein n may be any integer from 1 to 9. The NCM may include a plurality of (Cn) spacers.

In various embodiments of the chemically enhanced primer of the invention, the chemically-enhanced primer may have a structure of the formula: (Cn)ₓ-OLIGO , wherein (Cn)ₓ has a structure of the following formula:

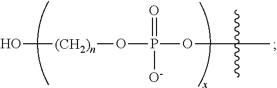

wherein each instance of n is independently an integer of 1 to 9; and x is an integer of 1 to about 30; OLIGO has a structure of the following formula:

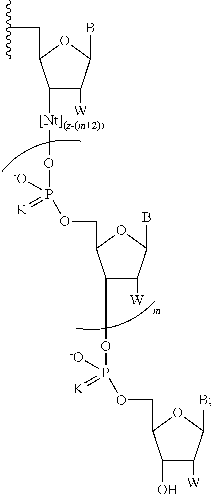

wherein B is a nucleobase; K is S or O; m is 0 or 1; z is an integer of 3 to about 100; W is OH, F, OMe, or H; and Nt is a moiety having a formula:

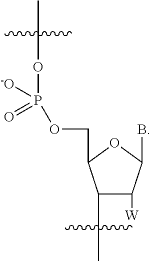

In another aspect of the invention, a kit is described which includes: a polymerase, a nuclease, at least one deoxynucleotide triphosphate, and dideoxynucleotide triphosphates. The dideoxynucleotide triphosphates may be dideoxynucleotide triphosphates labeled with a dye-label. The dye-labeled dideoxynucleotide triphosphates may be fluorescent dye-labeled dideoxynucleotide triphosphates.

In some embodiments, the nuclease may be selected from exonuclease I, Exo III, Pfu and DNA pol I. In various embodiments, the kit may include a chemically enhanced primer as described in this disclosure.

In other embodiments, the kit may further include a plurality of nuclease sensitive amplification primers. The plurality of nuclease- sensitive amplification primers may be configured to prime a sequence of interest of a specific disease state. The plurality of nuclease-sensitive amplification primers of the kit may be configured to prime a set of sequences connected to a specific disease state.

Various patents, patent applications, and other publications are referred to herein, all of which are incorporated herein in their entireties by reference. In addition, the following standard reference works are incorporated herein by reference: Current Protocols in Molecular Biology, John Wiley & Sons, N.Y., edition as of October 2007; Sambrook, Russell, and Sambrook, Molecular Cloning: A Laboratory Manual, 3rd ed., Cold Spring Harbor Laboratory Press, Cold Spring Harbor, 2001. In the event of a conflict between the instant specification and any document incorporated by reference, the specification shall control, it being understood that the determination of whether a conflict or inconsistency exists is within the discretion of the inventors and can be made at any time.

Additional features and advantages of the present teachings will be evident from the description that follows, and in part will be apparent from the description, or can be learned by practice of the present teachings. It is to be understood that both the foregoing summary and the following detailed description are exemplary and explanatory only and are intended to provide further explanation of the present teachings without limiting the present teachings.

### BRIEF DESCRIPTION OF THE DRAWINGS

The accompanying drawings, which are incorporated in and constitute a part of this specification exemplify embodiments disclosed and, together with the description, serve to explain and illustrate principles of disclosed embodiments. Specifically:

FIG. 1 is a graphical representation of the workflow for verifying a variant sequence via a capillary electrophoresis separation, using a small aliquot of preamplified sample. This permits the use of size-limited sample to be analyzed both in a NGS method, for example, but not limited to Ion AmpliSeq™ semiconductor sequencing, as well as confirmatory analysis via an efficient orthogonal capillary electrophoresis analysis pathway.

FIG. 2 is an annotated description of the analysis steps for processing the preamplified sample in a method of the invention.

FIG. 3 is a flowchart of the data analysis of the sequence data obtained via a method of the invention.

FIG. 4 is a schematic representation of the samples tested and the type of data. CE represents capillary electrophoresis (Sanger sequencing data) and PGM™ represents Ion Personal Genome Machine® (data is semiconductor sequencing data). CHP v2 is pre-amplification material derived from the Ion Torrent AmpliSeq™ Cancer Hot Spot Panel v2 and OCP is pre-amplificate from a proprietary Ion Torrent AmpliSeq OncoMine™.

FIG. 5 is a schematic representation of the specific targets of the verification assays performed by Sanger re-sequencing and in particular BigDye® Direct sequencing techniques. CHP v.2 indicates that those loci are part of the Ion AmpliSeq™ Cancer Hotspot Panel v.2 and OCP indicates that the indicated loci are part of the Ion Oncomine™ cancer panel.

FIG. 6 is a schematic representation of the variants found arising from three samples, using Ion AmpliSeq methodology on the Ion PGM™ (318 chip). The second column indicates the number of variants found in the specific sample. The remaining columns to the right indicate, for a specific loci, percentage observed for a variant sequence.

FIG. 7 is a schematic representation of the variant sequences found from the same three samples, upon resequencing using the methods of the invention, via Sanger sequencing. The same loci are interrogated and variants are confirmed.

FIGS. 8A-8B are schematic representations of the Quality Grid (as seen in Applied Biosystems Variant Reporter ™ software) for Target Sanger CE Test Set A for CHP v2 PA of FIG. 5. The lower panel of FIG. 8A is reproduced in larger scale in FIG. 8B, and demonstrates for each of four very limited originating samples taken through the workflow from AmpliSeq to Sanger Sequencing, that 88 out of 96 resulting amplicons have 2× coverage (fwd/rev), and 8 have 1× coverage. There are no drop outs. Right facing arrow indicates successful forward extension product production and left facing arrow indicates successful reverse extension product production.

FIGS. 9A-9B are schematic representations of the Quality Grid (as seen in Applied Biosystems Variant Reporter ™ software) for Target Sanger CE Test Set B for CHP v2 PA of FIG. 5. The lower panel of FIG. 9A is reproduced in larger scale in FIG. 9B, and demonstrates for each of four very limited originating samples taken through the workflow from AmpliSeq to Sanger Sequencing, that 93 out of 96 amplicons have 2× coverage (fwd/rev), and 3 have 1× coverage. There are no drop outs. Right facing arrow indicates successful forward extension product production and left facing arrow indicates successful reverse extension product production.

FIG. 10 is a graphical representation of the electropherogram demonstrating the sequencing results detecting a minor variant in ALK-2 for sample FFPE-5. The arrows in the left panel (forward sequence) and right panel (reverse sequence) clearly show a significant amount of minor variant under the major variant signal peak, which can be called by KB™ basecaller as a mixed base. This visual ratio can be compared to the ratio provided for the AmpliSeq derived results obtained by use of Ion Torrent Suite™ software to analyze the ratio of minor to major, which assigns a 26.8% ratio for the minor variant.

FIG. 11 is a graphical representation of the electropherogram demonstrating the sequencing results detecting a minor variant in EGFR-6 for sample NA 8020. The arrows in the left panel (forward sequence) and right panel (reverse sequence) clearly show a detectable amount of minor variant under the major variant signal peak, while it could not be called by KB™ basecaller as a mixed base. This visual ratio can be compared to the ratio provided for the AmpliSeq derived results obtained by use of Ion Torrent Suite™ software to analyze the ratio of minor to major, which assigns a 9.6% ratio for the minor variant.

FIG. 12 is a schematic representation of the frequency of TP53 mutations found from sequencing of three samples using OCP AmpliSeq™ on the Ion PGM™ (318 chip).

FIG. 13 is a schematic representation of the resequenced samples of FIG.12, using the methods of the invention to verify the TP53 mutations shown in FIG. 12.

FIGS. 14A-14B are graphical representations of the Quality Grid (as seen in Applied Biosystems Variant Reporter™ software) for 24 TP53 Individual Amplicons from OCP Ampliseq™, for four samples. The lower panel of FIG. 14A is reproduced in larger scale in FIG. 14B, and demonstrates for each of four very limited originating samples taken through the workflow from AmpliSeq™ to Sanger Sequencing, that 94 of 96 amplicons have complete 2× coverage (fwd/rev). There are no drop outs. Right facing arrow indicates successful forward extension product production and left facing arrow indicates successful reverse extension product production.

FIG. 15 is a graphical representation of the electropherogram of the sequencing results detecting a minor variant in TP53 for sample FFPE 5. The arrows in the left panel (forward sequence) and right panel (reverse sequence) clearly show a detectable amount of minor variant under the major variant signal peak. The use of Ion Torrent Suite™ software to analyze the ratio of minor (C) to major (T) assigns a 17.9% ratio for the minor variant.

FIG. 16 is a graphical representation of the electropherogram of the sequencing results detecting a minor variant in TP53 at a different position from that shown in FIG. 15, for sample FFPE 5. The arrows in the left panel (forward sequence) and right panel (reverse sequence) clearly show a detectable amount of minor variant under the major variant signal peak. The use of Ion Torrent Suite™ software to analyze the ratio of minor (T) to major (C) assigns a 21.8% ratio for the minor variant.

FIG. 17 is a graphical representation of the electropherogram of the sequencing results detecting a minor variant in TP53 at yet a third position from that shown in FIG. 15, for sample FFPE 5. The arrows in the left panel (forward sequence) and right panel (reverse sequence) clearly show a detectable amount of minor variant under the major variant signal peak. The use of Ion Torrent Suite™ software to analyze the ratio of minor (C) to major (G) assigns a 20.2% ratio for the minor variant.

### DETAILED DESCRIPTION

To facilitate understanding of the present teachings, the following definitions are provided. It is to be understood that, in general, terms not otherwise defined are to be given their ordinary meanings or meanings as generally accepted in the art.

As used herein, “amplify”, “amplifying” or “amplification reaction” and their derivatives, refer generally to any action or process whereby at least a portion of a nucleic add molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic: acid molecule. The template nucleic acid molecule can be single-stranded or double-stranded and the additional nucleic add molecule can independently be single-stranded or double-stranded. In some embodiments, amplification includes a template-dependent in vitro enzyme-catalyzed reaction for the production of at least one copy of at least some portion of the nucleic add molecule or the production of at least one copy of a nucleic acid sequence that is complementary to at least some portion of the nucleic add molecule. Amplification optionally includes linear or exponential replication of a nucleic add molecule. In some embodiments, such amplification is performed using isothermal conditions; in other embodiments, such amplification can include thermocycling. In some embodiments, the amplification k a multiplex amplification that includes the simultaneous amplification of a plurality of target sequences in a single amplification reaction. At least some of the target sequences can be situated on the same nucleic add molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes polymerase chain reaction (PCR).

As used herein, “amplification conditions” and its derivatives, generally refers to conditions suitable for amplifying one or more nucleic acid sequences. Such amplification can be linear or exponential. In some embodiments, the amplification conditions can include isothermal conditions or alternatively can include thermocycling conditions, or a combination of isothermal and thermocycling conditions. In some embodiments, the conditions suitable for amplifying one or more nucleic acid sequences includes polymerase chain reaction (PCR) conditions. Typically, the amplification conditions refer to a reaction mixture that is sufficient to amplify nucleic acids such as one or more target sequences, or to amplify an amplified target sequence ligated to one or more adapters, e.g., an adapter-ligated amplified target sequence. Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending and separating are repeated. Typically, the amplification conditions include cations such as Mg⁺⁺ or Mn⁺⁺ (e.g., MgCl₂, etc) and can also include various modifiers of ionic strength.

As used herein, “target sequence” or “sequence of interest” and its derivatives, refers generally and interchangeably to any single or double-stranded nucleic acid sequence that can be amplified or synthesized according to the disclosure, including any nucleic acid sequence suspected or expected to be present in a sample. In some embodiments, the sequence of interest is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

As defined herein, a “cleavable group” generally refers to any moiety that once incorporated into a nucleic acid can be cleaved under appropriate conditions. For example, a cleavable group can be incorporated into a target-specific primer, an amplified sequence, an adapter or a nucleic acid molecule of the sample. In an exemplary embodiment, a target-specific primer can include a cleavable group that becomes incorporated into the amplified product and is subsequently cleaved after amplification, thereby removing a portion, or all, of the target-specific primer from the amplified product. The cleavable group can be cleaved or otherwise removed from a target-specific primer, an amplified sequence, an adapter or a nucleic acid molecule of the sample by any acceptable means. For example, a cleavable group can be removed from a target-specific primer, an amplified sequence, an adapter or a nucleic acid molecule of the sample by enzymatic, thermal, photo-oxidative or chemical treatment. In one aspect, a cleavable group can include a nucleobase that is not naturally occurring. For example, an oligodeoxyribonucleotide can include one or more RNA nucleobases, such as uracil that can be removed by a uracil glycosylase. In some embodiments, a cleavable group can include one or more modified nucleobases (such as 7-methylguanine, 8-oxo-guanine, xanthine, hypoxanthine, 5,6-dihydrouracil or 5-methylcytosine) or one or more modified nucleosides (i.e., 7-methylguanosine, 8-oxo-deoxyguanosine, xanthosine, inosine, dihydrouridine or 5-methylcytidine). The modified nucleobases or nucleotides can be removed from the nucleic acid by enzymatic, chemical or thermal means. In one embodiment, a cleavable group can include a moiety that can be removed from a primer after amplification (or synthesis) upon exposure to ultraviolet light (i.e., bromodeoxyuridine). In another embodiment, a cleavable group can include methylated cytosine. Typically, methylated cytosine can be cleaved from a primer for example, after induction of amplification (or synthesis), upon sodium bisulfite treatment. In some embodiments, a cleavable moiety can include a restriction site. For example, a primer or target sequence can include a nucleic acid sequence that is specific to one or more restriction enzymes, and following amplification (or synthesis), the primer or target sequence can be treated with the one or more restriction enzymes such that the cleavable group is removed. Typically, one or more cleavable groups can be included at one or more locations with a target-specific primer, an amplified sequence, an adapter or a nucleic acid molecule of the sample.

As used herein, “cleavage step” and its derivatives, generally refers to any process by which a cleavable group is cleaved or otherwise removed from a target-specific primer, an amplified sequence, an adapter or a nucleic acid molecule of the sample. In some embodiments, the cleavage steps involves a chemical, thermal, photo-oxidative or digestive process.

The terms “complementary” and “complement” and their variants, as used herein, refer to any two or more nucleic acid sequences (e.g., portions or entireties of template nucleic acid molecules, target sequences and/or primers) that can undergo cumulative base pairing at two or more individual corresponding positions in antiparallel orientation, as in a hybridized duplex. Such base pairing can proceed according to any set of established rules, for example according to Watson-Crick base pairing rules or according to some other base pairing paradigm. Optionally there can be “complete” or “total” complementarity between a first and second nucleic acid sequence where each nucleotide in the first nucleic acid sequence can undergo a stabilizing base pairing interaction with a nucleotide in the corresponding antiparallel position on the second nucleic acid sequence. “Partial” complementarity describes nucleic acid sequences in which at least 20%, but less than 100%, of the residues of one nucleic acid sequence are complementary to residues in the other nucleic acid sequence. In some embodiments, at least 50%, but less than 100%, of the residues of one nucleic acid sequence are complementary to residues in the other nucleic acid sequence. In some embodiments, at least 70%, 80%, 90%, 95% or 98%, but less than 100%, of the residues of one nucleic acid sequence are complementary to residues in the other nucleic acid sequence. Sequences are said to be “substantially complementary” when at least 85% of the residues of one nucleic acid sequence are complementary to residues in the other nucleic acid sequence. In some embodiments, two complementary or substantially complementary sequences are capable of hybridizing to each other under standard or stringent hybridization conditions. “Non-complementary” describes nucleic acid sequences in which less than 20% of the residues of one nucleic acid sequence are complementary to residues in the other nucleic acid sequence. Sequences are said to be “substantially non-complementary” when less than 15% of the residues of one nucleic acid sequence are complementary to residues in the other nucleic acid sequence. In some embodiments, two non-complementary or substantially non-complementary sequences cannot hybridize to each other under standard or stringent hybridization conditions. A “mismatch” is present at any position in the two opposed nucleotides are not complementary. Complementary nucleotides include nucleotides that are efficiently incorporated by DNA polymerases opposite each other during DNA replication under physiological conditions. In a typical embodiment, complementary nucleotides can form base pairs with each other, such as the A-T/U and G-C base pairs formed through specific Watson-Crick type hydrogen bonding, or base pairs formed through some other type of base pairing paradigm, between the nucleobases of nucleotides and/or polynucleotides in positions antiparallel to each other. The complementarity of other artificial base pairs can be based on other types of hydrogen bonding and/or hydrophobicity of bases and/or shape complementarity between bases.

As used herein, “DNA barcode” or “DNA tagging sequence” and its derivatives, refers generally to a unique short (6-14 nucleotide) nucleic acid sequence within an adapter that can act as a ‘key’ to distinguish or separate a plurality of amplified target sequences in a sample. For the purposes of this disclosure, a DNA barcode or DNA tagging sequence can be incorporated into the nucleotide sequence of an adapter.

As used herein, “contacting” and its derivatives, when used in reference to two or more components, refers generally to any process whereby the approach, proximity, mixture or commingling of the referenced components is promoted or achieved without necessarily requiring physical contact of such components, and includes mixing of solutions containing any one or more of the referenced components with each other. The referenced components may be contacted in any particular order or combination and the particular order of recitation of components is not limiting.

As used herein, the term “determining a nucleotide base sequence” or the term “determining information about a sequence” encompasses “sequence determination” and also encompasses other levels of information such as eliminating one or more possibilities for a sequence. It is noted that performing sequence determination of a polynucleotide typically yields equivalent information regarding the sequence of a perfectly complementary (100% complementary) polynucleotide and thus is equivalent to sequence determination performed directly on a perfectly complementary polynucleotide.

As used herein, the term “end” and its variants, when used in reference to a nucleic acid molecule, for example a target sequence or amplified target sequence, can include the terminal 30 nucleotides, the terminal 20 and even more typically the terminal 15 nucleotides of the nucleic acid molecule. A linear nucleic acid molecule comprised of linked series of contiguous nucleotides typically includes at least two ends. In some embodiments, one end of the nucleic acid molecule can include a 3′ hydroxyl group or its equivalent, and can be referred to as the “3′ end” and its derivatives. Optionally, the 3′ end includes a 3′ hydroxyl group that is not linked to a 5′ phosphate group of a mononucleotide pentose ring. Typically, the 3′ end includes one or more 5′ linked nucleotides located adjacent to the nucleotide including the unlinked 3′ hydroxyl group, typically the 30 nucleotides located adjacent to the 3′ hydroxyl, typically the terminal 20 and even more typically the terminal 15 nucleotides. Generally, the one or more linked nucleotides can be represented as a percentage of the nucleotides present in the oligonucleotide or can be provided as a number of linked nucleotides adjacent to the unlinked 3′ hydroxyl. For example, the 3′ end can include less than 50% of the nucleotide length of the oligonucleotide. In some embodiments, the 3′ end does not include any unlinked 3′ hydroxyl group but can include any moiety capable of serving as a site for attachment of nucleotides via primer extension and/or nucleotide polymerization. In some embodiments, the term “3′ end” for example when referring to a target-specific primer, can include the terminal 10 nucleotides, the terminal 5 nucleotides, the terminal 4, 3, 2 or fewer nucleotides at the 3′end. In some embodiments, the term “3′ end” when referring to a target-specific primer can include nucleotides located at nucleotide positions 10 or fewer from the 3′ terminus.

As used herein, “5′ end”, and its derivatives, generally refers to an end of a nucleic acid molecule, for example a target sequence or amplified target sequence, which includes a free 5′ phosphate group or its equivalent. In some embodiments, the 5′ end includes a 5′ phosphate group that is not linked to a 3′ hydroxyl of a neighboring mononucleotide pentose ring. Typically, the 5′ end includes to one or more linked nucleotides located adjacent to the 5′ phosphate, typically the 30 nucleotides located adjacent to the nucleotide including the 5′ phosphate group, typically the terminal 20 and even more typically the terminal 15 nucleotides. Generally, the one or more linked nucleotides can be represented as a percentage of the nucleotides present in the oligonucleotide or can be provided as a number of linked nucleotides adjacent to the 5′ phosphate. For example, the 5′ end can be less than 50% of the nucleotide length of an oligonucleotide. In another exemplary embodiment, the 5′ end can include about 15 nucleotides adjacent to the nucleotide including the terminal 5′ phosphate. In some embodiments, the 5′ end does not include any unlinked 5′ phosphate group but can include any moiety capable of serving as a site of attachment to a a 3′ hydroxyl group, or to the 3′end of another nucleic acid molecule. In some embodiments, the term “5′ end” for example when referring to a target-specific primer, can include the terminal 10 nucleotides, the terminal 5 nucleotides, the terminal 4, 3, 2 or fewer nucleotides at the 5′end. In some embodiments, the term “5′ end” when referring to a target-specific primer can include nucleotides located at positions 10 or fewer from the 5′ terminus. In some embodiments, the 5′ end of a target-specific primer can include only non-cleavable nucleotides, for example nucleotides that do not contain one or more cleavable groups as disclosed herein, or a cleavable nucleotide as would be readily determined by one of ordinary skill in the art.

As used herein, the term “hybridization” is consistent with its use in the art, and generally refers to the process whereby two nucleic acid molecules undergo base pairing interactions. Two nucleic acid molecule molecules are said to be hybridized when any portion of one nucleic acid molecule is base paired with any portion of the other nucleic acid molecule; it is not necessarily required that the two nucleic acid molecules be hybridized across their entire respective lengths and in some embodiments, at least one of the nucleic acid molecules can include portions that are not hybridized to the other nucleic acid molecule. The phrase “hybridizing under stringent conditions” and its variants refers generally to conditions under which hybridization of a target-specific primer to a target sequence occurs in the presence of high hybridization temperature and low ionic strength. In one exemplary embodiment, stringent hybridization conditions include an aqueous environment containing about 30 mM magnesium sulfate, about 300 mM Tris-sulfate at pH 8.9, and about 90 mM ammonium sulfate at about 60-68° C., or equivalents thereof. As used herein, the phrase “standard hybridization conditions” and its variants refers generally to conditions under which hybridization of a primer to an oligonucleotide (i.e., a target sequence), occurs in the presence of low hybridization temperature and high ionic strength. In one exemplary embodiment, standard hybridization conditions include an aqueous environment containing about 100 mM magnesium sulfate, about 500 mM Tris-sulfate at pH 8.9, and about 200 mM ammonium sulfate at about 50-55° C., or equivalents thereof.

As used herein, the terms “ligating”, “ligation” and their derivatives refer generally to the act or process for covalently linking two or more molecules together, for example, covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, for example embodiments wherein the nucleic acid molecules to be ligated include conventional nucleotide residues, the litigation can include forming a covalent bond between a 5′ phosphate group of one nucleic acid and a 3′ hydroxyl group of a second nucleic acid thereby forming a ligated nucleic acid molecule. In some embodiments, any means for joining nicks or bonding a 5′phosphate to a 3′ hydroxyl between adjacent nucleotides can be employed. In an exemplary embodiment, an enzyme such as a ligase can be used. Generally for the purposes of this disclosure, an amplified target sequence can be ligated to an adapter to generate an adapter-ligated amplified target sequence.

As used herein, “ligase” and its derivatives, refers generally to any agent capable of catalyzing the ligation of two substrate molecules. In some embodiments, the ligase includes an enzyme capable of catalyzing the joining of racks between adjacent nucleotides of a nucleic add. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases may include, but not limited to, T4 DNA ligase, T4 RNA ligase, and E. coli DNA ligase.

As used herein, “blunt-end ligation” and its derivatives, refers generally to ligation of two blunt-end double-stranded nucleic acid molecules to each other. A “blunt end” refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion greater than two nucleotides in length, referred to herein as an “overhang”. In some embodiments, the end of nucleic acid molecule does not include any single stranded portion, such that every nucleotide in one strand of the end is based paired with opposing nucleotides in the other strand of the same nucleic acid molecule. In some embodiments, the ends of the two blunt ended nucleic acid molecules that become ligated to each other do not include any overlapping, shared or complementary sequence. Typically, blunted-end ligation excludes the use of additional oligonucleotide adapters to assist in the ligation of the double-stranded amplified target sequence to the double-stranded adapter, such as patch oligonucleotides as described in Mitra and Varley, US2010/0129874, published May 27, 2010. In some embodiments, blunt-ended ligation includes a nick translation reaction to seal a nick created during the ligation process.

As used herein, the terms “adapter” or “adapter and its complements” and their derivatives, refers generally to any linear oligonucleotide which can be ligated to a nucleic acid molecule of the disclosure. Optionally, the adapter includes a nucleic acid sequence that is not substantially complementary to the 3′ end or the 5′ end of at least one target sequences within the sample. In some embodiments, the adapter is substantially non-complementary to the 3′ end or the 5′ end of any target sequence present in the sample. In some embodiments, the adapter includes any single stranded or double-stranded linear oligonucleotide that is not substantially complementary to an amplified target sequence. In some embodiments, the adapter is substantially non-complementary to at least one, some or all of the nucleic acid molecules of the sample. In some embodiments, suitable adapter lengths are in the range of about 10-100 nucleotides, about 12-60 nucleotides and about 15-50 nucleotides in length. Generally, the adapter can include any combination of nucleotides and/or nucleic acids. In some aspects, the adapter can include one or more cleavable groups at one or more locations. In another aspect, the adapter can include a sequence that is substantially identical, or substantially complementary, to at least a portion of a primer, for example a universal primer. In some embodiments, the adapter can include a barcode or tag to assist with downstream cataloguing, identification or sequencing. In some embodiments, a single-stranded adapter can act as a substrate for amplification when ligated to an amplified target sequence, particularly in the presence of a polymerase and dNTPs under suitable temperature and pH.

As used herein, the term “polymerase chain reaction” (“PCR”) refers to the method of K. B. Mullis U.S. Pat. Nos. 4,683,195 and 4,683,202, hereby incorporated by reference, which describe a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a precise sequence of thermal cycling in the presence of a DNA polymerase. The two primers are complementary to their respective strands of the double stranded polynucleotide of interest. To effect amplification, the mixture is denatured and the primers then annealed to their complementary sequences within the polynucleotide of interest molecule. Following annealing, the primers are extended with a polymerase to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified”. As defined herein, target nucleic acid molecules within a sample including a plurality of target nucleic acid molecules are amplified via PCR. In a modification to the method discussed above, the target nucleic acid molecules can be PCR amplified using a plurality of different primer pairs, in some cases, one or more primer pairs per target nucleic acid molecule of interest, thereby forming a multiplex PCR reaction. Using multiplex PCR, it is possible to simultaneously amplify multiple nucleic acid molecules of interest from a sample to form amplified target sequences. It is also possible to detect the amplified target sequences by several different methodologies (e.g., quantitation with a bioanalyzer or qPCR, hybridization with a labeled probe; incorporation of biotinylated primers followed by avidin-enzyme conjugate detection; incorporation of ³²P-labeled deoxynucleotide triphosphates, such as dCTP or dATP, into the amplified target sequence). Any oligonucleotide sequence can be amplified with the appropriate set of primers, thereby allowing for the amplification of target nucleic acid molecules from genomic DNA, cDNA, formalin-fixed paraffin-embedded DNA, fine-needle biopsies and various other sources. In particular, the amplified target sequences created by the multiplex PCR process as disclosed herein, are themselves efficient substrates for subsequent PCR amplification or various downstream assays or manipulations.

As defined herein “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexy” or “plex” of a given multiplex amplification refers generally to the number of different target-specific sequences that are amplified during that single multiplex amplification. In some embodiments, the plexy can be about 12-plex, 24-plex, 48-plex, 96-plex, 192-plex, 384-plex, 768-plex, 1536-plex, 3072-plex, 6144-plex or higher.

“Cycle sequencing” as used herein, refers to a process that includes adding to a target nucleic acid or an amplification product thereof, sequencing primer, deoxynucleotide triphosphates (dNTPs), dye-labeled chain terminating nucleotides (e.g.,dideoxynucleotide triphosphates (ddNTPs-dyes)), and DNA polymerase, followed by thermal cycle sequencing. Standard cycle sequencing procedures are well established. Cycle sequencing procedures are described in more detail, for example, in U.S. Pat. No. 5,741,676, and U.S. Pat. No. 5,756,285, each hereby incorporated by reference in its entirety. In certain embodiments, “cycle sequencing” comprises dNTPS, a sequencing primer (labeled or not), ddNTPs (labeled or not) and DNA polymerase as known to one of skill in the art. It is noted that a labeled sequencing primer can provide fragment analysis information and/or determination of the sequence of a target nucleic acid or amplification product thereof.

As used herein, the term “PCR/cycle sequencing” refers to a method for determining a nucleotide sequence of DNA by PCR amplifying the DNA, followed by sequencing reactions repeated (or cycled) several times. This cycling is similar to PCR because the sequencing reaction is allowed to proceed at a preselected temperature where polymerase extension may occur, i.e. 42° C.-55° C., then extension is stopped by heating to 95° C., and finally the cycle is started again at 42° C.-55° C. Cycle sequencing uses a thermostable DNA polymerase.

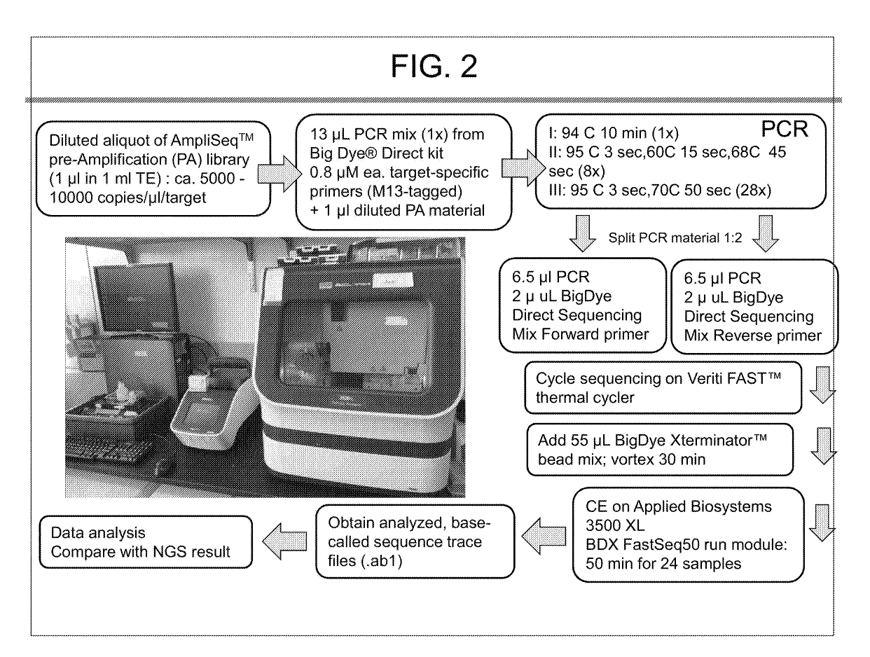

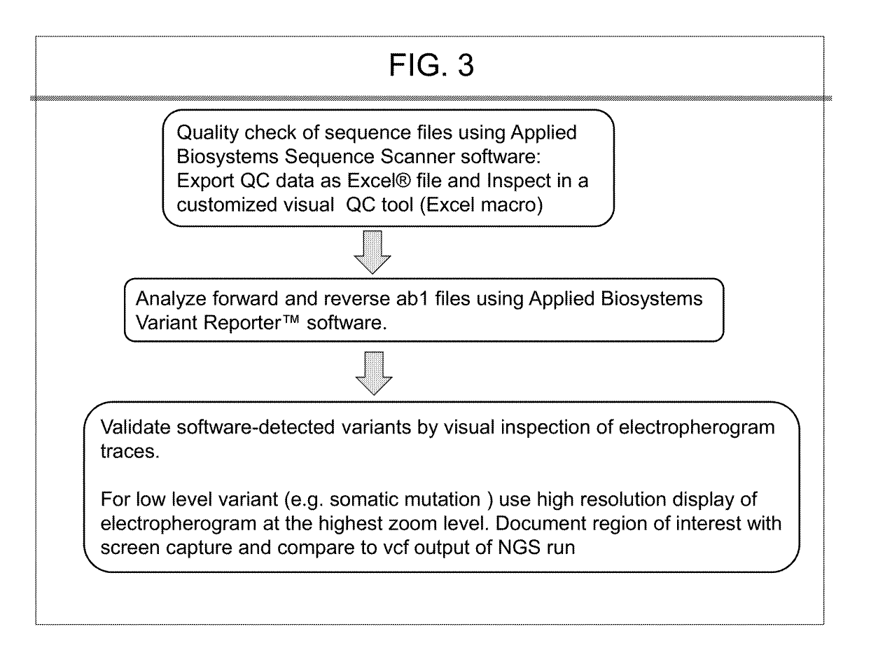

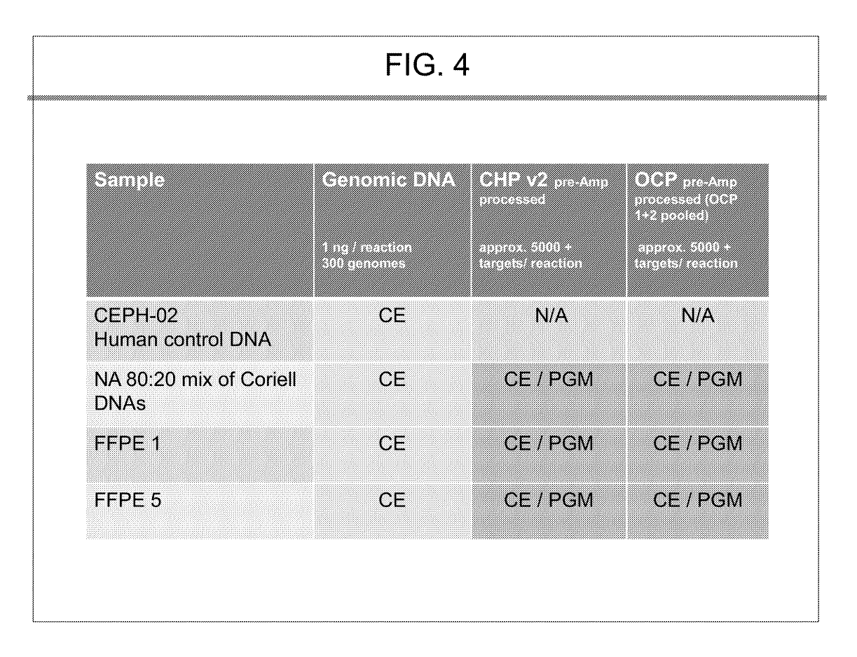

As used herein, the term “phosphorothioate linkage” refers to an inter-nucleotide linkage comprising a sulfur atom in place of a non-bridging oxygen atom within the phosphate linkages of a sugar phosphate backbone. The term phosphorothioate linkage refers to both phosphorothioate inter-nucleotide linkages and phosphorodithioate inter-nucleotide linkages. A “phosphorothioate linkage at a terminal 3′ end” refers to a phosphorothioate linkage at the 3′ terminus, that is, the last phosphate linkage of the sugar phosphate backbone at the 3′ terminus. A phosphorothioate linkage at a terminal 3′ end is illustrated in FIG. 2.

As used herein, the term “phosphodiester linkage” may refer to the linkage—PO₄—which is used to link nucleotide monomers, such as the inter-nucleotide linkages found in naturally-occurring DNA. Additionally, “phosphodiester linkage” may refer to portions of the NCMs or NCM linkers of the chemically-enhanced primers of the present disclosure.

As used herein, the term “nuclease-resistant linkage” refers to an oligonucleotide sequence, such as a primer, that is resistant to digestion in the 3′ to 5′ direction by nuclease. Phosphorothioate and boronophosphate linkages are two examples of nuclease-resistant linkages. The examples are not to be construed as limiting to just these examples.

As used herein, the term “primer” and its derivatives refer generally to any polynucleotide that can hybridize to a target sequence of interest. In some embodiments, the primer can also serve to prime nucleic acid synthesis. Typically, the primer functions as a substrate onto which nucleotides can be polymerized by a polymerase; in some embodiments, however, the primer can become incorporated into the synthesized nucleic acid strand and provide a site to which another primer can hybridize to prime synthesis of a new strand that is complementary to the synthesized nucleic acid molecule. The primer may be comprised of any combination of nucleotides or analogs thereof, which may be optionally linked to form a linear polymer of any suitable length. In some embodiments, the primer is a single-stranded oligonucleotide or polynucleotide. (For purposes of this disclosure, the terms ‘polynucleotide” and “oligonucleotide” are used interchangeably herein and do not necessarily indicate any difference in length between the two). In some embodiments, the primer is single-stranded but it can also be double-stranded. The primer optionally occurs naturally, as in a purified restriction digest, or can be produced synthetically. In some embodiments, the primer acts as a point of initiation for amplification or synthesis when exposed to amplification or synthesis conditions; such amplification or synthesis can occur in a template-dependent fashion and optionally results in formation of a primer extension product that is complementary to at least a portion of the target sequence. Exemplary amplification or synthesis conditions can include contacting the primer with a polynucleotide template (e.g., a template including a target sequence), nucleotides and an inducing agent such as a polymerase at a suitable temperature and pH to induce polymerization of nucleotides onto an end of the target-specific primer. If double-stranded, the primer can optionally be treated to separate its strands before being used to prepare primer extension products. In some embodiments, the primer is an oligodeoxyribonucleotide or an oligoribonucleotide. In some embodiments, the primer can include one or more nucleotide analogs. The exact length and/or composition, including sequence, of the target-specific primer can influence many properties, including melting temperature (Tm), GC content, formation of secondary structures, repeat nucleotide motifs, length of predicted primer extension products, extent of coverage across a nucleic acid molecule of interest, number of primers present in a single amplification or synthesis reaction, presence of nucleotide analogs or modified nucleotides within the primers, and the like. In some embodiments, a primer can be paired with a compatible primer within an amplification or synthesis reaction to form a primer pair consisting or a forward primer and a reverse primer. In some embodiments, the forward primer of the primer pair includes a sequence that is substantially complementary to at least a portion of a strand of a nucleic acid molecule, and the reverse primer of the primer of the primer pair includes a sequence that is substantially identical to at least of portion of the strand. In some embodiments, the forward primer and the reverse primer are capable of hybridizing to opposite strands of a nucleic acid duplex. Optionally, the forward primer primes synthesis of a first nucleic acid strand, and the reverse primer primes synthesis of a second nucleic acid strand, wherein the first and second strands are substantially complementary to each other, or can hybridize to form a double-stranded nucleic acid molecule. In some embodiments, one end of an amplification or synthesis product is defined by the forward primer and the other end of the amplification or synthesis product is defined by the reverse primer. In some embodiments, where the amplification or synthesis of lengthy primer extension products is required, such as amplifying an exon, coding region, or gene, several primer pairs can be created than span the desired length to enable sufficient amplification of the region. In some embodiments, a primer can include one or more cleavable groups. In some embodiments, primer lengths are in the range of about 10 to about 60 nucleotides, about 12 to about 50 nucleotides and about 15 to about 40 nucleotides in length. Typically, a primer is capable of hybridizing to a corresponding target sequence and undergoing primer extension when exposed to amplification conditions in the presence of dNTPS and a polymerase. In some instances, the particular nucleotide sequence or a portion of the primer is known at the outset of the amplification reaction or can be determined by one or more of the methods disclosed herein. In some embodiments, the primer includes one or more cleavable groups at one or more locations within the primer.

As used herein, “target-specific primer” and its derivatives, refers generally to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the target sequence itself; in other embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the nucleic acid molecule other than the target sequence. In some embodiments, the target-specific primer is substantially non-complementary to other target sequences present in the sample; optionally, the target-specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target-specific primer is designed to include a nucleotide sequence that is substantially complementary to at least a portion of its corresponding target sequence. In some embodiments, a target-specific primer is at least 95% complementary, or at least 99% complementary, or identical, across its entire length to at least a portion of a nucleic acid molecule that includes its corresponding target sequence. In some embodiments, a target-specific primer can be at least 90%, at least 95% complementary, at least 98% complementary or at least 99% complementary, or identical, across its entire length to at least a portion of its corresponding target sequence. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In some embodiments, the target-specific primer can be substantially non-complementary at its 3’ end or its 5′ end to any other target-specific primer present in an amplification reaction. In some embodiments, the target-specific primer can include minimal cross hybridization to other target-specific primers in the amplification reaction. In some embodiments, target-specific primers include minimal cross-hybridization to non-specific sequences in the amplification reaction mixture. In some embodiments, the target-specific primers include minimal self-complementarity. In some embodiments, the target-specific primers can include one or more cleavable groups located at the 3′ end. In some embodiments, the target-specific primers can include one or more cleavable groups located near or about a central nucleotide of the target-specific primer. In some embodiments, one of more targets-specific primers includes only non-cleavable nucleotides at the 5′ end of the target-specific primer. In some embodiments, a target specific primer includes minimal nucleotide sequence overlap at the 3′end or the 5′ end of the primer as compared to one or more different target-specific primers, optionally in the same amplification reaction. In some embodiments 1, 2, 3, 4, 5, 6, 7, 8, 9, 10 or more, target-specific primers in a single reaction mixture include one or more of the above embodiments. In some embodiments, substantially all of the plurality of target-specific primers in a single reaction mixture includes one or more of the above embodiments.

As used herein, the term “chemically-enhanced primer” refers to a primer that can have a negatively charged moiety at a terminal 5′ end of the primer or within the primer. The primer can also include a nuclease-resistant linkage at the last phosphate linkage of the sugar phosphate backbone at the 3′ terminus.

As used herein, the term “sequencing primer” refers to an oligonucleotide primer that is used to initiate a sequencing reaction performed on a nucleic acid. The term “sequencing primer” refers to both a forward sequencing primer and to a reverse sequencing primer.

As used herein, the term “extension primer” refers to an oligonucleotide, capable of annealing to a nucleic acid region adjacent a target sequence, and serving as an initiation primer for elongation of the oligonucleotide by using the target sequence as the complementary template for nucleotide extension under suitable conditions well known in the art. Typically, a sequencing reaction employs at least one extension primer or a pair of extension primers. The pair would include an “upstream” or “forward” primer and a “downstream” or “reverse” primer, which delimit a region of the nucleic acid target sequence to be sequenced.

As used herein, the term “amplification primer” refers to an oligonucleotide, capable of annealing to an RNA or DNA region adjacent a target sequence, and serving as an initiation primer for nucleic acid synthesis under suitable conditions well known in the art. Typically, a PCR reaction employs a pair of amplification primers including an “upstream” or “forward” primer and a “downstream” or “reverse” primer, which delimit a region of the RNA or DNA to be amplified.

As used herein, the term “tailed primer” or “tailed amplification primer” or “tailed sequencing primer” refers to a primer that includes at its 3′end a sequence capable of annealing to an RNA or DNA region adjacent a target sequence, and serving as an initiation primer for DNA synthesis under suitable conditions well known in the art. The primer includes at its 5′end a sequence that is not complementary to the target sequence.

The term “extension” and its variants, as used herein, when used in reference to a given primer, comprises any in vivo or in vitro enzymatic activity characteristic of a given polymerase that relates to polymerization of one or more nucleotides onto an end of an existing nucleic acid molecule. Typically but not necessarily such primer extension occurs in a template-dependent fashion; during template-dependent extension, the order and selection of bases is driven by established base pairing rules, which can include Watson-Crick type base pairing rules or alternatively (and especially in the case of extension reactions involving nucleotide analogs) by some other type of base pairing paradigm. In one non-limiting example, extension occurs via polymerization of nucleotides on the 3′OH end of the nucleic acid molecule by the polymerase.

The term “nucleic acid sequence” as used herein can refer to the nucleic acid material itself and is not restricted to the sequence information (i.e. the succession of letters chosen among the five base letters A, C, G, T, or U) that biochemically characterizes a specific nucleic acid, for example, a DNA or RNA molecule. Nucleic acids shown herein are presented in a 5′→3′ orientation unless otherwise indicated.

The term “mobility-dependent separation” as used herein can refer to the separation of nucleic acid fragments due to the charge and size associated with the fragment.

The term “fluorescent dye” as used herein refers to moieties that absorb light energy at a defined excitation wavelength and emit light energy at a different wavelength. Preferably the fluorescent dyes selected for use are spectrally resolvable. As used herein, “spectrally resolvable” means that the dyes can be distinguished on the basis of their spectral characteristics, particularly fluorescence emission wavelength, under conditions of operation. For example, the identity of the one or more terminal nucleotides can be correlated to a distinct wavelength of maximum light emission intensity, or perhaps a ratio of intensities at different wavelengths.

The term “nucleobase” or “base” as used herein refers to a nitrogen-containing heterocyclic moiety capable of forming Watson-Crick type hydrogen bonds with a complementary nucleobase or nucleobase analog, e.g. a purine, a 7-deazapurine, or a pyrimidine. Typical nucleobases are the naturally occurring nucleobases adenine, guanine, cytosine, 5mC, uracil, thymine, and analogs of naturally occurring nucleobases, e.g. 7-deazaadenine, 7-deaza-8-azaadenine, 7-deazaguanine, 7-deaza-8-azaguanine, N6-Δ2 isopentenyl-adenine(6iA), N6-Δ2-isopentenyl-2-methylthioadenine (2ms6iA), N2-dimethyl-guanine(dmG), 7-methylguanine (7mG), inosine, nebularine, nitropyrrole, nitroindole, 2-amino-purine, 2,6-diamino-purine, hypoxanthine, pseudouridine, pseudocytidine, pseudoisocytidine, 5-propynyl-cytidine, isocytidine, isoguanine, 2-thiopyrimidine, 6-thioguanine, 4-thiothymine, 4-thiouracil, O⁶-methylguanine, N⁶-methyl-adenine, O⁴-methylthymine, 5,6-dihydrothymine, 5,6-dihydrouracil, 4-methylindole, pyrazolo[3,4-D]pyrimidines (see, e.g., U.S. Pat. Nos. 6,143,877 and 6,127,121 and PCT Published Application WO 01/38584)and ethenoadenine. Nonlimiting examples of nucleotide bases can be found, e.g., in Fasman, Practical Handbook of Biochemistry and Molecular Biology, pp. 385-394, CRC Press, Boca Raton, Fla. (1989).

As used herein, the term “nucleotide” and its variants comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. In some embodiments, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH₃, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281. In some embodiments, the nucleotide comprises a label and referred to herein as a “labeled nucleotide”; the label of the labeled nucleotide is referred to herein as a “nucleotide label”. In some embodiments, the label can be in the form of a fluorescent dye attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. .alpha.-thio-nucleotide 5′-triphosphates. For a review of nucleic acid chemistry, see: Shabarova, Z. and Bogdanov, A. Advanced Organic Chemistry of Nucleic Acids, VCH, New York, 1994.

As used herein, the terms “polynucleotide”, “nucleic acid”, or “oligonucleotide” refers to a linear polymer of nucleosides (including deoxyribonucleosides, ribonucleosides, or analogs thereof) joined by inter-nucleosidic linkages. Whenever a polynucleotide such as an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes deoxythymidine, unless otherwise noted. The letters A, C, G, and T can be used to refer to the bases themselves, to nucleosides, or to nucleotides comprising the bases, as is standard in the art. In naturally occurring polynucleotides, the inter-nucleoside linkage is typically a phosphodiester bond, and the subunits are referred to as “nucleotides.” Oligonucleotide primers comprising other inter-nucleoside linkages, such as phosphorothioate linkages, are used in certain embodiments of the teachings. It will be appreciated that one or more of the subunits that make up such an oligonucleotide primer with a non-phosphodiester linkage may not comprise a phosphate group. Such analogs of nucleotides are considered to fall within the scope of the term “nucleotide” as used herein, and nucleic acids comprising one or more inter-nucleoside linkages that are not phosphodiester linkages are still referred to as “polynucleotides”, “oligonucleotides”, etc.

As used herein, “polymerase” and its derivatives, generally refers to any enzyme that can catalyze the polymerization of nucleotides (including analogs thereof) into a nucleic acid strand. Typically but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer based polymerase that optionally can be reactivated.

As defined herein, “sample” and its derivatives, is used in its broadest sense and includes any specimen, culture and the like that is suspected of including a target. In some embodiments, the sample comprises DNA, RNA, PNA, LNA, chimeric, hybrid, or multiplex-forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen.

As used herein “sequence determination”, “determining a nucleotide base sequence”, “sequencing”, and like terms includes determination of partial as well as full sequence information. That is, the term includes sequence comparisons, fingerprinting, and like levels of information about a target polynucleotide, as well as the express identification and ordering of each nucleoside of the target polynucleotide within a region of interest. In certain embodiments, “sequence determination” comprises identifying a single nucleotide, while in other embodiments more than one nucleotide is identified. Identification of nucleosides, nucleotides, and/or bases are considered equivalent herein. It is noted that performing sequence determination on a polynucleotide typically yields equivalent information regarding the sequence of a perfectly complementary polynucleotide and thus is equivalent to sequence determination performed directly on a perfectly complementary polynucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, primer set(s), etc. in the appropriate containers) and/or supporting materials (e.g., buffers, written instructions for performing the assay etc.) from one location to another. For example, kits can include one or more enclosures (e.g., boxes) containing the relevant reaction reagents and/or supporting materials. As used herein, the term “fragmented kit” refers to a delivery system comprising two or more separate containers that each contain a subportion of the total kit components. The containers may be purchased and/or delivered to the intended recipient together or separately. For example, a first container may contain an enzyme for use in an assay, while a second container contains oligonucleotides. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As will be appreciated by one of ordinary skill in the art, references to templates, oligonucleotides, primers, etc., generally mean populations or pools of nucleic acid molecules that are substantially identical within a relevant region rather than single molecules. For example, a “template” generally means a plurality of substantially identical template molecules; a “primer” generally means a plurality of substantially identical primer molecules, and the like.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Methods for verification of NGS sequencing results. The introduction of defined Ion AmpliSeq™ panels, amongst other commercially available assays using Next Generation Sequencing (NGS) techniques for detection and characterization of actionable mutations occurring in tumor tissue has the potential to revolutionize translational oncology research. This technology is further described in U.S. Application Ser. Nos. 61/479,952, filed on Apr. 28, 2011; 61/531,583, filed on Sep. 6, 2011; 61/531,574, filed on Sep. 6, 2011; 61/538,079, filed on Sep. 22, 2011; 61/564,763, filed on Nov. 29, 2011; 61/578,192, filed on Dec. 20, 2011; 61/594,160, filed on Feb. 2, 2012; 61/598,881, filed on Feb. 14, 2012; 61/598,892, filed on Feb. 14, 2012; 61/625,596, filed on Apr. 17, 2012; 61/639,017, filed on Apr. 26, 2012; Ser. No. 13/458,739, filed on Apr. 27, 2012; Ser. No. 13/663,334, filed on Oct. 29, 2012; Ser. No. 13/679,706, filed on Nov. 16, 2012; and an application entitled “Detection, Identification, Validation, and Enrichment of Target Nucleic Acids”, Attorney Docket No. LT00974 PRO, filed on even date; and each disclosure is hereby incorporated by reference in its entirety.

The Ion Ampliseq™ cancer hot spot panel version 2 (CHP v2) by Ion Torrent includes 207 actionable mutation targets present in 50 genes and the more comprehensive Ion Oncomine™ cancer panel (OCP) developed by Life Technologies Compendia Bioscience™ contains over 2000 mutations. A hallmark of these Ion Torrent Ampliseq cancer panels is the low amount of input DNA needed which is critical when the clinical specimen material is limited such as with fine needle biopsy, aspirates, LCM or FFPE samples. Typically, 10 ng of DNA obtained from these sources is sufficient to produce informative sequencing data. Often, cancer-causing or promoting mutations are detected at relatively low allele frequencies like 10-20% compared to the major normal allele.

New methods are needed to verify these findings of low frequency mutations by an orthologous method such as traditional dye-fluorescent Sanger sequencing on a capillary electrophoresis (CE) instrument such as the Applied Biosystems 3500 genetic analyzer.

A workflow that enables the amplification and Sanger sequencing of individual Ion AmpliSeq targets directly from the Ampliseq™ library starting material is described here. This workflow can also be used with library starting materials arising out of other Next Generation Sequencing (NGS) methods of massively parallel sequencing.

The method requires a retainer of 1 μl (˜5%) of the original Ampliseq™ preamplification material. A dilution of this aliquot is used as template source for individualized PCR/sequencing reactions. A random selection of 48 targets from the CHPv2 panel may be successfully amplified and Sanger-sequenced from an Ion Torrent Ampliseq™ library originally prepared from 10 ng of FFPE DNA. Furthermore, the successful Sanger-re-sequencing of all individual 24 targets covering the TP53 exons from the same sample processed and pre-amplified with the OncoMine AmpliSeq panel.

Taken together, this method permits reflex-test of potential mutations of interest from very material-limited specimen using Sanger CE sequencing. It provides a reflex solution for verifying and following up NGS results by Sanger sequencing particularly for samples with very limited amounts of available DNA, such as samples obtained from any of fine needle biopsies, aspirates, formalin-fixed, paraffin-embedded (FFPE), and Laser Capture Microdissection (LCM).

Additionally, this workflow offers other advantages over typical Sanger sequencing protocols, removing extra manipulations and purifications. This streamlining also is advantageous when working with quantity limited samples. For example, a typical PCR reaction uses an excess of amplification primers, some primers remain unincorporated upon completion of the PCR reaction. This necessitates removal of the excess primers before proceeding to a sequencing reaction, because the excess amplification primers will interfere with the subsequent sequencing reaction, and may produce aberrant sequence ladders. The PCR reaction furthermore contains an excess of dNTPs that can interfere with the subsequent sequencing reaction. In the current workflow, addition of a nuclease to the sequencing reaction mixture before the start of the cycle sequencing reaction, which nuclease may be but is not limited to exonuclease I, utilizes its hydrolytic properties to degrade single-stranded DNA present in the PCR mixture, thus allowing the amplification product (amplicon) to be used more efficiently in the subsequent sequencing reaction.

Resolution of nucleic acid sequence near the sequencing primer had been difficult in the past to obtain without sacrificing throughput residence time during electrophoresis. Adjustments in the type of mobility system, adjusting denaturing conditions and temperature can improve resolution but always at the expense of increased electrophoresis time. Difficulties in removal of unincorporated reactants and long residence time when performing size-dependent mobility separation contributed to inefficiencies in nucleic acid sequencing. There are several advantages of the improved BigDye® Direct amplification/sequencing workflow that addresses these problems. Only one post synthesis cleanup is needed; it can be performed very easily in the same reactor vessel. Additionally, the nature of the chemically enhanced sequencing primer produces extension products that can be more easily detected without contamination and complicating signal from excess reagent, without slowing the electrophoresis experiment.

What has been surprising discovered by Applicant is that Ampliseq™ primer design is transferable to Sanger CE sequencing. Using the advanced chemistry of BigDye Direct sequencing which streamlines the workflow as described here and in the cross-referenced applications, allows simpler, less time intensive sequencing analysis which also has very high 5′ resolution. Use of M13 tags for target specific nuclease sensitive amplification primers permits the use of M13 chemically enhanced sequencing primers, which survive in situ nuclease degradation of excess PCR amplification primers before the start of sequence fragment production. Additionally, the other modifications of the M13 chemically enhanced sequencing primers allows basecalling to begin at base number 1 of the sequence of interest. Various aspects of the use of chemically-enhanced sequencing primers and the combined steps of the Sanger sequencing workflow are further described in U.S. Application Ser. Nos. 61/026,085, filed Feb. 4, 2008; Ser. No. 12/365,140, filed Feb. 3, 2009; 61/407,899, filed Oct. 28, 2010; 61/408,553, filed Oct. 29, 2010; Ser. No. 13/284,839, filed Oct. 28, 2011; and Ser. No. 13/397,626, filed Feb. 15, 2012, and each disclosure of which is hereby incorporated by reference in its entirety.

Applicant has also surprisingly found that 1 ng of genomic DNA is sufficient for Fwd/Rev pair of sequences from a single target. Further, pre-amplification (PA) material from low complexity Ampliseq™ panels (i.e. CHP v2 and OCP) can be diluted and used as template source for re-PCR and Sanger sequencing.

High complexity Ampliseq™ panels (CCP and whole exome) has not been attempted yet for re-PCR and sequencing, but may afford access.

A method for sequencing at least one amplicon is provided which includes the steps of: providing at least one amplicon, wherein the at least one amplicon comprises a sequence of interest and a preceding sequence 5′ to the sequence of interest incorporated from a first priming sequence; amplifying the at least one amplicon in a first reaction mixture which includes a plurality of nuclease-sensitive amplification primers to form an amplified DNA product; contacting the first reaction mixture containing the amplified DNA product with a second reaction mixture comprising a nuclease and at least one chemically-enhanced primer causing the plurality of nuclease sensitive amplification primers to be degraded by the nuclease; inactivating the nuclease; priming the amplified DNA product with the at least one chemically-enhanced primer in a sequencing reaction; and producing extension products of the at least one chemically enhanced primer. In some embodiments, the extension products may be fluorescently labeled. In various embodiments, the first priming sequence may have been used to produce the amplicon. In various embodiments, the first priming sequence may include at least one cleavable moiety. In some embodiments, the preceding sequence may be a portion of the first priming sequence. In various embodiments of the method, the steps of contacting the first reaction mixture with the second reaction mixture, inactivating the nuclease, and producing the extension products of the chemically enhanced primer may be performed in the same reaction vessel. In various embodiments, the steps of amplifying the at least one amplicon, contacting the first reaction mixture with the second reaction mixture, inactivating the nuclease, and producing the extension products of the chemically enhanced primer may be performed without intermediate purification steps.

In various embodiments of the method, the at least one amplicon further includes a succeeding sequence 3′ to the sequence of interest wherein the succeeding sequence is complementary to a second priming sequence used to produce the at least one amplicon. The at least one amplicon may have a length of about 100 nucleotides to about 400 nucleotides. The sequence of interest of the at least one amplicon may have a length of about 100 nucleotides to about 300 nucleotides. In other embodiments, the sequence of interest of the at least one amplicon may have a length of about 125 nucleotides to about 275 or about 250 nucleotides. The at least one amplicon may be a plurality of amplicons. In some embodiments, the plurality of amplicons may include at least two different amplicons, a first having a sequence of interest that is a major variant sequence and a second amplicon having a minor variant sequence from the same region of a sample nucleic acid.

In some embodiments, the method further includes the steps of obtaining sequencing results based on the sequencing reaction; and determining a nucleotide base sequence of at least the sequence of interest based on the results. The sequencing results may be obtained via a mobility based separation method. In some embodiments, the mobility based separation method may be capillary electrophoresis. In some embodiments, the determined nucleotide base sequence of at least the sequence of interest may be compared to a second nucleotide base sequence of at least the sequence of interest obtained from a NGS method of sequencing. The NGS method of sequencing may include massively parallel sequencing techniques like sequencing by synthesis using fluorophore or semiconductor detection and pyrosequencing, to name a few. In some embodiments, the NGS method of sequencing may be semiconductor sequencing.

In various embodiments, amplifying DNA may include polymerase chain reaction amplification. In selected embodiments, the sequencing reaction may include cycle sequencing.

In various embodiments, the first reaction mixture may also include a polymerase. The polymerase may be a thermostable polymerase. In some embodiments, the polymerase may be Taq polymerase. The first reaction mixture may further include deoxynucleotide triphosphates.

In various embodiments, the second reaction mixture further comprises a polymerase, deoxynucleotide triphosphates, and dye-labelled dideoxynucleotide triphosphates. The polymerase of the second reaction mixture may be a thermostable polymerase. In some embodiments, the polymerase is Taq polymerase.

In various embodiments of the method for sequencing at least one amplicon, the nuclease may be selected from exonuclease I, Exo III, Pfu and DNA pol I.

In various embodiments of the method for sequencing at least one amplicon, each of the plurality of nuclease-sensitive amplification primers may be configured to prime a sequence of interest of a specific disease state. In some embodiments, the plurality of nuclease-sensitive amplification primers may prime a set of sequences connected to a specific disease state.

Another method is described for confirming a DNA sequence, which includes the steps of: amplifying a sample comprising nucleic acid using at least a first priming sequence to provide a plurality of amplicons, where each of the plurality of amplicons includes a sequence of interest and a preceding sequence 5′ to the sequence of interest incorporated from a first priming sequence; amplifying a first aliquot of the plurality of amplicons in a first reaction mixture including a plurality of nuclease-sensitive amplification primers to form an amplified DNA product; contacting the first reaction mixture containing the amplified DNA product with a second reaction mixture that includes a nuclease and at least one chemically-enhanced primer, where by contacting the nuclease with the first reaction mixture, the nuclease sensitive amplification primers are degraded by the nuclease; inactivating the nuclease; priming the amplified DNA product with the at least one chemically-enhanced primer in a sequencing reaction; and producing extension products of the chemically enhanced primer. In some embodiments, the extension products may be fluorescently labeled. In various embodiments, the first priming sequence may have been used to produce the amplicon. The first priming sequence may include at least one cleavable moiety. In some embodiments, the preceding sequence may be a portion of the first priming sequence.

In various embodiments of the method, the steps of contacting the first reaction mixture with the second reaction mixture, inactivating the nuclease, and producing the extension products of the chemically enhanced primer may be performed in the same reaction vessel. In various embodiments, the steps of amplifying the plurality of amplicons, contacting the first reaction mixture with the second reaction mixture, inactivating the nuclease, and producing the extension products of the chemically enhanced primer may be performed without intermediate purification steps.

In various embodiments of the method, each of the plurality of amplicons further includes a succeeding sequence 3′ to the sequence of interest wherein the succeeding sequence is complementary to a second priming sequence used to produce the amplicon. Each of the plurality of amplicons may have a length of about 100 nucleotides to about 400 nucleotides. The sequence of interest of each of the plurality of amplicons may have a length of about 100 nucleotides to about 300 nucleotides. In other embodiments, the sequence of interest of each of the plurality of amplicons may have a length of about 125 nucleotides to about 275 or about 250 nucleotides. In some embodiments, the plurality of amplicons may include at least two different amplicons, a first having a sequence of interest that is a major variant sequence and a second amplicon having a minor variant sequence from the same region of a sample nucleic acid.

In some embodiments, the method further includes the steps of obtaining sequencing results based on the sequencing reaction; and determining a nucleotide base sequence of at least the sequence of interest based on the results. The sequencing results may be obtained via a mobility based separation method. In some embodiments, the mobility based separation method may be capillary electrophoresis. In some embodiments, the determined nucleotide base sequence of at least the sequence of interest may be compared to a second nucleotide base sequence of at least the sequence of interest obtained from a NGS method of sequencing performed on a second aliquot of the plurality of amplicons. The NGS method of sequencing may include massively parallel sequencing techniques like sequencing by synthesis using fluorophore or semiconductor detection and pyrosequencing, to name a few. In some embodiments, the NGS method of sequencing may be semiconductor sequencing.

In various embodiments, amplifying DNA may include polymerase chain reaction amplification. In selected embodiments, the sequencing reaction may include cycle sequencing.

In various embodiments, the first reaction mixture may also include a polymerase. The polymerase may be a thermostable polymerase. In some embodiments, the polymerase may be Taq polymerase. The first reaction mixture may further include deoxynucleotide triphosphates.

In various embodiments, the second reaction mixture further comprises a polymerase, deoxynucleotide triphosphates, and dye-labelled dideoxynucleotide triphosphates. The polymerase of the second reaction mixture may be a thermostable polymerase. In some embodiments, the polymerase is Taq polymerase.

In various embodiments of the method for confirming a DNA sequence, the nuclease may be selected from exonuclease I, Exo III, Pfu and DNA pol I.

In various embodiments of the method for confirming a DNA sequence, each of the plurality of nuclease-sensitive amplification primers may be configured to prime a sequence of interest of a specific disease state. In some embodiments, the plurality of nuclease-sensitive amplification primers may prime a set of sequences connected to a specific disease state.

Another method is provided for preparing DNA for sequencing, including the steps of: amplifying a sample comprising nucleic acid using at least a first priming sequence to provide a plurality of amplicons, where each of the plurality of amplicons includes a sequence of interest and a preceding sequence 5′ to the sequence of interest incorporated from a first priming sequence; amplifying an aliquot of the plurality of amplicons in a first reaction mixture which includes nuclease-sensitive amplification primers to form an amplified DNA product; contacting the first reaction mixture containing the amplified DNA product with a second reaction mixture comprising a nuclease and a chemically-enhanced primer where by contacting the nuclease with the first reaction mixture, the nuclease sensitive amplification primers are degraded by the nuclease; inactivating the nuclease; priming the amplified DNA product with the chemically-enhanced primer in a sequencing reaction; and producing extension products of the chemically enhanced primer.

Yet another method is described for sequencing and verifying a variant nucleic acid sequence of interest, including the steps: amplifying a sample which includes nucleic acid using at least a first priming sequence to provide a plurality of amplicons, where each of the plurality of amplicons includes a sequence of interest and a preceding sequence 5′ to the sequence of interest incorporated from a first priming sequence; splitting the plurality of amplicons into a first aliquot and a second aliquot; amplifying the first aliquot of the plurality of amplicons in a first reaction mixture including nuclease-sensitive amplification primers to form a first amplified DNA product; contacting the first reaction mixture containing the first amplified DNA product with a second reaction mixture which includes a nuclease and a chemically-enhanced primer where by contacting the nuclease with the first reaction mixture, the nuclease sensitive amplification primers are degraded by the nuclease; inactivating the nuclease; priming the first amplified DNA product with the chemically-enhanced primer in a sequencing reaction; producing extension products of the chemically enhanced primer; obtaining sequencing results of at least the sequence of interest of the extended chemically enhanced primer using a mobility dependent separation; and determining a nucleotide base sequence of at least the sequence of interest of the extended chemically enhanced primer; amplifying the second aliquot of the amplicons to form a second DNA product; obtaining sequencing results of at least the sequence of interest of the second DNA product using a NGS sequencing method; and verifying a nucleotide sequence of the second DNA product by comparing it to the nucleotide base sequence of at least the sequence of interest of the extended chemically enhanced primer. In various embodiments of the method, the step of amplifying the second aliquot of the plurality of amplicons to form a second DNA product further comprises at least one of ligating adaptors, binding to beads, and ligating barcodes.

For any of the methods described above and throughout this disclosure, the nucleic acid can also be amplified using other methods such as, for example, multiple strand displacement amplification, helicase displacement amplification, nick translation, Q beta replicase amplification, rolling circle amplification, and other isothermal amplification methods. The nucleic acid to be amplified can comprise, for example, RNA, DNA, cDNA, genomic DNA, viral DNA, plasmid DNA, recombinant DNA, amplicon DNA, synthetic DNA or the like.

For any of the methods described above and throughout this disclosure, templates to be sequenced can be synthesized by PCR in individual aqueous compartments (also called “reactors”) of an emulsion. In some embodiments, the compartments can each contain a particulate support such as a bead having a suitable first amplification primer attached thereto, a first copy of the template, a second amplification primer, and components needed for a PCR reaction (for example nucleotides, polymerase, cofactors, and the like). Methods for preparing emulsions are described, for example, in U.S. Pat. No. 6,489,103 B1, U.S. Pat. No. 5,830,663, and in U.S. Patent Application Publication No. US 2004/0253731. Methods for performing PCR within individual compartments of an emulsion to produce clonal populations of templates attached to microparticles are described, for example, in Dressman, D., et al, Proc. Natl. Acad. Sci., 100(15):8817-8822, 2003, and in PCT publication WO2005010145. All of the patents, applications, publications, and articles described herein are incorporated in their entireties by reference.

According to various embodiments, the amplification primers can comprise tailed primers. The tailed primers can be used, for example, to generate a target specific amplicon that incorporates nucleic acid sequence capable of annealing to a universal primer or a gene specific primer.

For any of the methods described above and throughout this disclosure, nucleases suitable for use in the subject methods preferentially degrade single-stranded polynucleotides over double-stranded polynucleotides, thus destroying excess primers while leaving intact double-stranded amplicons available for sequencing in subsequent steps. In various embodiments, the nuclease enzyme can comprise, for example, exonuclease I. Exonuclease I can be obtained from various commercial suppliers, for example from USB Corp., Cleveland, Ohio. Appropriate reaction conditions can include, for example, optimal time, temperature, and buffer parameters to provide for nuclease enzyme activity. In some embodiments, for example, excess amplification primer can be degraded by adding exonuclease Ito the amplification reaction product and incubating at about 37° C. for about 10 to about 30 min. Exonuclease I can hydrolyze single-stranded DNA in a 3′→5′ direction. The exonuclease I can be sensitive to heat inactivation and can be essentially 100 percent deactivated by heating, for example, heating at about 80° C. for about 15 minutes. Other heat inactivated nucleases may be used in the subject methods and compositions including but not limited to Exo III, Pfu or DNA pol I. In various embodiments, the inactivation of the nuclease can occur within the vesicle and in the same reaction step as the sequencing reaction

The chemically-enhanced sequencing primer can be essentially non-degraded by a reaction mixture comprising a nuclease, for example, exonuclease I, under reaction conditions at which excess amplification primer can be degraded by the nuclease. By “essentially non-degraded” it is intended that any degradation that takes place of the chemically-enhanced sequencing primer is not of a level that significantly interferes with the process employed to generate sequencing and/or fragment analysis data in the subsequent sequencing reactions or fragment analysis reactions. In some embodiments, the chemically-enhanced sequencing primer can comprise one of more nuclease-resistant internucleotide linkage(s). For example, the internucleotide linkage may be a phosphorothioate linkage. In some embodiments, the chemically-enhanced sequencing primer can comprise a nuclease-resistant internucleotide linkage at a terminal 3′ end, at a terminal 5′ end, and/or at one or more internal linkage sites. In some embodiments, the nuclease resistant internucleotide linkage is at least one phosphorothioate linkage. Chemically-enhanced sequencing primers were synthesized having one or two phosphorothioate linkages on the terminal 3′ end to protect the chemically-enhanced sequencing primers from exonuclease I digestion. The Sp stereoisomer can protect the primer from exonuclease I digestion but the Rp steroisomer was found to provide no protection from exonuclease I digestion (data not shown).

For any of the methods described above and throughout this disclosure, the mobility-dependent separation is selected from separation by charge and separation by size, wherein the separation by size plus charge is selected from gel electrophoresis and capillary electrophoresis and separation by size is by a liquid gradient, and a denaturing gradient medium. I he sequencing reaction products can be analyzed on a sieving or non-sieving medium. In some embodiments of these teachings, for example, the PCR products can be analyzed by electrophoresis; e.g., capillary electrophoresis, as described in H. Wenz et al. (1998), GENOME RES. 8:69-80 (see also E. Buel et al. (1998), J. FORENSIC SCI. 43:(1), pp. 164-170)), or slab gel electrophoresis, as described in M. Christensen et al. (1999), SCAND. J. CLIN. LAB. INVEST. 59(3): 167-177, or denaturing polyacrylamide gel electrophoresis (see, e.g., J. Sambrook et al. (1989), in MOLECULAR CLONING: A LABORATORY MANUAL, SECOND EDITION, Cold Spring Harbor Laboratory Press, Cold Spring Harbor, N.Y., pp. 13.45-13.57). The separation of DNA fragments in electrophoresis is based primarily on differential fragment size. Sequencing reaction products can also be analyzed by chromatography; e.g., by size exclusion chromatography (SEC). Likewise, fragment analysis can be carried in a similar manner as would be known to the skilled artisan.

For any of the methods described above and throughout this disclosure, each of the ddNTPs can be labeled with a different fluorescent dye (ddNTP-dye). For example, the ddNTPs can comprise BigDye® ddNTPs, available from Applied Biosystems, Foster City, Calif. In some embodiments, the chemically-enhanced primer can be labeled with a fluorescent dye. The label can be attached to the oligonucleotide sequence and/or the NCM region of the chemically-enhanced primer.

For any of the methods described above and throughout this disclosure, the chemically-enhanced primer may include an oligonucleotide sequence, a NCM and none or at least one nuclease-resistant linkage. In some embodiments, the chemically-enhanced primer may include one nuclease-resistant linkage at a terminal 3′ end. The chemically-enhanced primer may include a plurality of NCMs either at a terminal 5′ end or within a oligonucleotide sequence of the chemically-enhanced primer. In some embodiments the plurality of NCMs may be at a terminal 5′ end. In various embodiments, the NCM may be a (Cn) spacer wherein n is any integer from 1 to 9. The NCM may include a plurality of (Cn) spacers. In various embodiments, the chemically-enhanced primer may have a structure of the formula: (Cn)ₓ-OLIGO , where (Cn)ₓ has a structure of the following formula:

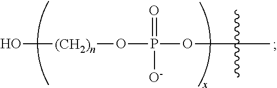

where each instance of n may independently be an integer of 1 to 9; and x may be an integer of 1 to about 30;
OLIGO has a structure of the following formula:

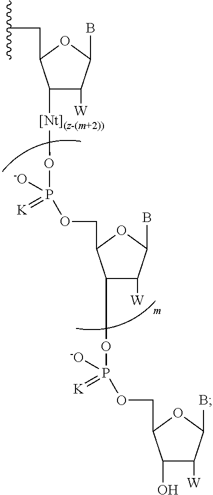

where B is a nucleobase; K is S or O; m is 0 or 1; z is an integer of 3 to about 100; W is OH, F, OMe, or H; and Nt is a moiety having a formula:

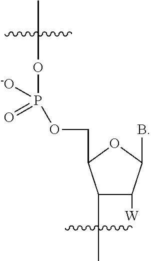

In some embodiments, the chemically enhanced primer may have any structure as described in this disclosure.

Other types of chemically-enhanced primers can be utilized within the scope of the present teachings. For example, a nuclease resistant sequencing primer can comprise an alkyl phosphonate monomer, RO—P(═O)(—Me)(—OR), such as dA-Me-phosphonamidite, and/or a triester monomer, RO—P(═O)(—OR′)(—OR), such as dA-Me-phophoramidite (available from Glen Research, Sterling, Va.), and/or a locked nucleic acid monomer (available from Exiqon, Woburn, Mass.), and/or a boranophosphate monomer, RO—P(—BH₃)(═O)(—OR), as described by Shaw, Barbara Ramsey, et al., in “Synthesis of Boron-Containing ADP and GDP Analogues: Nucleoside 5′-(P-Boranodisphosphates)”, Perspectives in Nucleoside and Nucleic Acid Chemistry, pg. 125-130, (2000), or the like.

In another method, one or more chemically-enhanced primers may be used for ligation extension reactions. In some embodiments, the chemically-enhanced primer for use in a ligation extension reaction is labeled fluorescently. In some embodiments, the ligation extension chemically-enhanced primer is labeled fluorescently at a 3′ terminus.

Polymerases useful in the methods. A variety of nucleic acid polymerases may be used in the methods described herein. For example, the nucleic acid polymerizing enzyme can be a thermostable polymerase or a thermally degradable polymerase. Suitable thermostable polymerases include, but are not limited to, polymerases isolated from Thermus aquaticus, Thermus thermophilus, Pyrococcus woesei, Pyrococcus furiosus, Thermococcus litoralis, and Thermotogo maritima. Suitable thermodegradable polymerases include, but are not limited to, E. coli DNA polymerase I, the Klenow fragment of E. coli DNA polymerase I, T4 DNA polymerase, T5 DNA polymerase, T7 DNA polymerase, and others. Examples of other polymerizing enzymes that can be used in the methods described herein include T7, T3, SP6 RNA polymerases and AMV, M-MLV and HIV reverse transcriptases.

Non-limiting examples of commercially available polymerases that can be used in the methods described herein include, but are not limited to, TaqFS®, AmpliTaq® CS (Applied Biosystems), AmpliTaq FS (Applied Biosystems), AmpliTaq Gold ® (Applied Biosystems), Kentaq1 (AB Peptide, St. Louis, Mo.), Taquenase (ScienTech Corp., St. Louis, Mo.), ThermoSequenase (Amersham), Bst polymerase, Vent_{R}(exo⁻) DNA polymerase, Reader™ Taq DNA polymerase, VENT™ DNA polymerase (New England Biolabs), DEEPVENT™ DNA polymerase (New England Biolabs), PFUTurbo™ DNA polymerase (Stratagene), Tth DNA polymerase, KlenTaq-1 polymerase, SEQUENASE™ 1.0 DNA polymerase (Amersham Biosciences), and SEQUENASE 2.0 DNA polymerase (United States Biochemicals).

Uses for the methods. Optionally, the method further includes detecting and/or identifying mutations present in the sample identified through nucleic acid sequencing of the amplified target sequence.

In some embodiments, target sequences or amplified target sequences are directed to mutations associated with cancer. In some embodiments, the target sequences or amplified target sequences are directed to mutations associated with one or more cancers selected from the group consisting of head and neck cancers, brain cancer, breast cancer, ovarian cancer, cervical cancer, colorectal cancer, endometrial cancer, gallbladder cancer, gastric cancer, bladder cancer, prostate cancer, testicular cancer, liver cancer, lung cancer, kidney (renal cell) cancer, esophageal cancer, pancreatic cancer, thyroid cancer, bile duct cancer, pituitary tumor, wilms tumor, kaposi sarcoma, osteosarcoma, thymus cancer, skin cancer, heart cancer, oral and larynx cancer, leukemia, neuroblastoma and non-hodgkin lymphoma. In one embodiment, the mutations can include substitutions, insertions, inversions, point mutations, deletions, mismatches and translocations. In one embodiment, the mutations can include variation in copy number. In one embodiment, the mutations can include germline or somatic mutations. In one embodiment, the mutations associated with cancer are located in at least one of the genes provided in Tables 1 or 4 of U.S. Patent Publication 20120295819, or provided in Table 7 of U.S. Application No. 61/598,881, each hereby incorporated by reference in its entirety. In some embodiments, the mutations can be any of the genomic coordinates provided in Table 5 of U.S. Patent Publication 20120295819, or provided in Table 7 of U.S. Application 61/598,881, each hereby incorporated by reference in its entirety. In some embodiments, the target sequences directed to mutations associated with cancer can include any one or more of the mutations provided in Table 10 of U.S. Patent Publication 20120295819, hereby incorporated by reference in its entirety. In some embodiments, the mutations can be found within any one or more of the genomic coordinates provided in Table 16 or Table 18 of U.S. Patent Publication 20120295819, hereby incorporated by reference in its entirety.

In some embodiments, the mutations associated with cancer are located in at least one of the genes selected from ABI1; ABL1; ABL2; ACSL3; ACSL6; AFF1; AFF3; AFF4;AKAP9; AKT1; AKT2; ALK; APC; ARHGAP26; ARHGEF12; ARID1A; ARNT; ASPSCR1; ASXL1; ATF1; ATIC; ATM; AXIN2; BAP1; BARD1; BCAR3; BCL10; BCL11A; BCL11B; BCL2; BCL3; BCL6; BCL7A;BCL9; BCR; BIRC3; BLM; BMPR1A; BRAF; BRCA1; BRCA2; BRD3; BRD4; BRIP1; BUB1B; CARD11; CARS; CASC5; CBFA2T3; CBFB; CBL; CBLB; CBLC; CCDC6; CCNB1IP1; CCND1; CCND2; CD74; CD79A; CDC73; CDH1; CDH11; CDK4; CDK6; CDKN2A; CDKN2B; CDKN2C; CDX2; CEBPA; CEP110; CHEK1; CHEK2; CHIC2; CHN1; CIC; CIITA; CLP1; CLTC; CLTCL1; COL1A1; CREB1; CREB3L2; CREBBP; CRTC1; CRTC3; CSF1R; CTNNB1; CXCR7; CYLD; CYTSB; DCLK3; DDB2; DDIT3; DDR2; DDX10; DDX5; DDX6; DEK; DGKG; DICER1; DNMT3A; EGFR; EIF4A2; ELF4; ELL; ELN; EML4; EP300; EPS15; ERBB2; ERBB4; ERC1; ERCC2; ERCC3; ERCC4; ERCC5; ERG; ETV1; ETV4; ETV5; ETV6; EWSR1; EXT1; EXT2; EZH2; FAM123B; FANCA; FANCC; FANCD2; FANCE; FANCF; FANCG; FAS; FBXW7; FCRL4; FGFR1; FGFR1OP; FGFR2; FGFR3; FH; FIP1L1; FLCN; FLI1; FLT1; FLT3; FNBP1; FOXL2; FOXO1; FOXO3; FOXO4; FOXP1; FUS; GAS7; GATA1; GATA2; GATA3; GMPS; GNAQ; GNAS; GOLGA5; GOPC; GPC3; GPHNGPR124; HIP1; HIST1H41; HLF; HNF1A; HNRNPA2B1; HOOK3; HOXA11; HOXA13; HOXA9; HOXC11; HOXC13; HOXD13; HRAS; HSP90AA1; HSP90AB1; IDH1; IDH2; IKZF1; IL2; IL21R; IL6ST; IRF4; ITGA10; ITGA9; ITK; JAK1; JAK2; JAK3; KDM5A; KDM5C; KDM6A; KDR; KDSR; KIAA1549; KIT; KLF6; KLK2; KRAS; KTN1; LASP1; LCK; LCP1; LHFP; LIFR; LMO2; LPP; MAF; MALT1; MAML2; MAP2K1; MAP2K4; MDM2; MDM4; MECOM; MEN1; MET; MITF; MKL1; MLH1; MLL; MLLT1; MLLT10; MLLT3; MLLT4; MLLT6; MN1; MPL; MRE11A; MSH2; MSH6; MSI2; MSN; MTCP1; MTOR; MUC1; MYB; MYC; MYCL1; MYCN; MYH11; MYH9; MYST3; MYST4; NACA; NBN; NCOA1; NCOA2; NCOA4; NEK9; NF1; NF2; NFE2L2; NFKB2; NIN; NKX2-1; NLRP1; NONO; NOTCH1; NOTCH2; NPM1; NR4A3; NRAS; NSD1; NTRK1; NTRK3; NUMA1; NUP214; NUP98; OLIG2; OMD; PAFAH1B2; PALB2; PATZ1; PAX3; PAX5; PAX7; PAX8; PBRM1; PBX1; PCM1; PDE4DIP; PDGFB; PDGFRA; PDGFRB; PER1; PHOX2B; PICALM; PIK3CA; PIK3R1; PIM1; PLAG1; PML; PMS1; PMS2; POU2AF1; POU5F1; PPARG; PPP2R1A; PRCC; PRDM16; PRF1; PRKAR1A; PRRX1; PSIP1; PTCH1; PTEN; PTPN11; RABEP1; RAD50; RAD51L1; RAF1; RANBP17; RAP1GDS1; RARA; RB1; RBM15; RECQL4; REL; RET; RHOH; RNF213; ROS1; RPN1; RPS6KA2; RUNX1; RUNX1T1; SBDS; SDHAF2; SDHB; SETD2; SFPQ; SFRS3; SH3GL1; SLC45A3; SMAD4; SMARCA4; SMARCB1; SMO; SOCS1; SRC; SRGAP3; SS18; SS18L1; STIL; STK11; STK36; SUFU; SYK; TAF15; TAF1L; TAL1; TAL2; TCF12; TCF3; TCL1A; TET1; TET2; TEX14; TFE3; TFEB; TFG; TFRC; THRAP3; TLX1; TLX3; TMPRSS2; TNFAIP3; TOP1; TP53; TPM3; TPM4; TPR; TRIM27; TRIM33; TRIP11; TSC1; TSC2; TSHR; USP6; VHL; WAS; WHSC1L1; WRN; WT1; XPA; XPC; ZBTB16; ZMYM2; ZNF331; ZNF384; and ZNF521.

In some embodiments, the mutations associated with cancer are located in at least one of the genes selected from ABL1; AKT1; ALK; APC; ATM; BRAF; CDH1; CDKN2A; CSF1R; CTNNB1; EGFR; ERBB2; ERBB4; FBXW7; FGFR1; FGFR2; FGFR3; FLT3; GNAS; HNF1A; HRAS; IDH1; JAK2; JAK3; KDR; KIT; KRAS; MET; MLH1; MPL; NOTCH1; NPM1; NRAS; PDGFRA; PIK3CA; PTEN; PTPN11; RB1; RET; SMAD4; SMARCB1; SMO; SRC; STK11; TP53; and VHL.

In some embodiments, the amplified target sequences are directed to any one of more of the genomic coordinates provided in Tables 5, 7 or 18 of U.S. Patent Publication 20120295819, hereby incorporated by reference in its entirety. In some embodiments, any one or more of the cancer target-specific primers provided in Tables 2, 3, 6 or 17 of U.S. Patent Publication 20120295819, hereby incorporated by reference in its entirety, can be used to amplify a target sequence present in a sample as disclosed by the methods described herein.

In some embodiments, the cancer target-specific primers from Tables 2, 3, 6, or 17 of U.S. Patent Publication 20120295819, hereby incorporated by reference in its entirety, can include 2, 3, 4, 5, 6, 7, 8, 9, 10, 20, 40, 60, 80, 100, 150, 200, 400, 500, 800, 1000, 2000, 3000, 4000, 5000, 6000, 7000, 8000, 9000, 10,000, 11,000, 12,000, 13,000 or more, target-specific primers. In some embodiments, the amplified target sequences can include any one or more of the amplified target sequences generated at the genomic coordinates (using amplicon ID target-specific primers) provided in Tables 5, 7, 10 or 18 of U.S. Patent Publication 20120295819, hereby incorporated by reference in its entirety. In some embodiments, at least one of the target-specific primers associated with cancer is at least 90% identical to at least one nucleic acid sequence selected from SEQ ID NOs: 1-103,143 of U.S. Patent Publication 20120295819, hereby incorporated by reference in its entirety. In some embodiments, at least one of the target-specific primers associated with cancer is complementary across its entire length to at least one target sequence in a sample. In some embodiments, at least one of the target-specific primers associated with cancer includes a non-cleavable nucleotide at the 3′ end. In some embodiments, the non-cleavable nucleotide at the 3′ end includes the terminal 3′ nucleotide. In one embodiment, the amplified target sequences are directed to individual exons having a mutation associated with cancer. In some embodiments, the disclosure relates generally to the selective amplification of more than one target sequences in a sample and the detection and/or identification of mutations associated with cancer. In some embodiments, the amplified target sequences include two or more nucleotide sequences provided in Table 2 of U.S. Patent Publication 20120295819, hereby incorporated by reference in its entirety. In some embodiments, the amplified target sequences can include any one or more the amplified target sequences generated at the genomic coordinates using the amplicon ID target-specific primers provided in Table 5 of U.S. Patent Publication 20120295819, or provided in Table 7 of US Application 61/598,881, each of which is hereby incorporated by reference in its entirety. In one embodiment, the amplified target sequences include 100, 200, 500, 1000, 2000, 3000, 6000, 8000, 10,000, 12,000, or more amplicons from Tables 1-5 of U.S. Patent Publication 20120295819, or Tables 6 and 7 of US Application 61/598,881 hereby incorporated by reference in their entireties. In some embodiments, the disclosure relates generally to the detection and optionally, the identification of clinically actionable mutations. As defined herein, the term “clinically actionable mutations” includes mutations that are known or can be associated by one of ordinary skill in the art with, but not limited to, prognosis for the treatment of cancer. In one embodiment, prognosis for the treatment of cancer includes the identification of mutations associated with responsiveness or non-responsiveness of a cancer to a drug, drug combination, or treatment regime. In one embodiment, the disclosure relates generally to the amplification of a plurality of target sequences from a population of nucleic acid molecules linked to, or correlated with, the onset, progression or remission of cancer.

In some embodiments, target-specific primers are designed using the primer criteria disclosed herein. In some embodiments, target-specific primers are designed using the primer criteria disclosed herein and directed to one or more genes associated with breast cancer. In some embodiments, target-specific primers associated with breast cancer include at least one target-specific primer selected from one or more genes selected from the group consisting of AIM1, AR, ATM, BARD1, BCAS1, BRIP1, CCND1, CCND2, CCNE1, CDH1, CDK3,CDK4,CDKN2A, CDKN2B, CAMK1D, CHEK2, DIRAS3, EGFR, ERBB2, EPHA3, ERBB4, ETV6, GNRH1, KCTD9, CDCA2, EBF2, EMSY, BNIP3L, PNMA2, DPYSL2, ADRA1A, STMN4, TRIM35, PAK1, AQP11, CLSN1A, RSF1, KCTD14, THRSP, NDUFC2, ALG8, KCTD21, USP35, GAB2, DNAH9, ZNF18, MYOCD, STK11, TP53, JAK1, JAK2, MET, PDGFRA, PML, PTEN, RET, TMPRSS2, WNK1, FGFR1, IGF1R, PPP1R12B, PTPRT, GSTM1, IPO8, MYC, ZNF703, MDM1, MDM2, MDM4,MKK4, P14KB, NCOR1, NBN, PALB2, RAD50, RAD51, PAK1,RSF1, INTS4, ZMIZ1, SEPHS1, FOXM1, SDCCAG1, IGF1R, TSHZ2, RPSK6K1, PPP2R2A, MTAP, MAP2K4, AURKB, BCL2, BUB1, CDCA3, CDCA4, CDC20, CDC45, CHEK1, FOXM1, HDAC2, IGF1R, KIF2C, KIFC1, KRAS, RB1, SMAD4, NCOR1, UTX, MTHDFD1L, RAD51AP1, TTK and UBE2C.

In some embodiments, the disclosure relates generally to the amplification of target sequences directed to mutations associated with a congenital or inherited disease. In some embodiments, the disclosure can include the amplification of target sequences directed to somatic or germline mutations. In some embodiments, the mutations can be autosomal dominant or autosomal recessive. In one embodiment, the mutations associated with a congenital or inherited disease are located in at least one of the genes or diseases provided in Table 4 of U.S. Patent Publication 20120295819, hereby incorporated by reference in its entirety. In some embodiments, the disclosure relates to the amplification of target sequences in a sample associated with one or more inherited diseases selected from the group consisting of Adenosine Aminohydrolase Deficiency (ADA); Agammaglobulinemia, X-linked, Type 1; Alagille Syndrome; All Hypertrophic and Dilated Cardiomyopathy; Alopecia Universalis Congenita (ALUNC); Alpers Syndrome; Alpha-1-Antitrypsin Deficiency; Alpha-Thalassemia-Southeast Asia; Amyotrophic Lateral Sclerosis-Lou Gehrig's Disease; Androgen Insensitivity Syndrome; Aniridia; Ankylosing spondylitis; APC-Associated Polyposis Conditions; Argininosuccinate Lyase Deficiency; Arrhythmogenic Right Ventricular Dysplasia/Cardiomyopathy; Ataxia with Oculomotor Apraxia Type 2; Ataxia with Vitamin E Deficiency; Ataxia-Telangiectasia; Autoimmune Polyendocrine Syndrome; Beta-Hydroxyisobutyryl CoA Deacylase deficiency (HIBCH deficiency); Biotinidase Deficiency; Blepharophimosis-ptosis-epicanthus inversus; Bloom Syndrome; Brachydactyly; Brachydactyly-Hypertension Syndrome; Brachydactyly Type B1; Branchiootorenal Spectrum Disorders; BRCA1; Campomelic Dysplasia; Canavan; Cerebrotendinous Xanthomatosis; Ceroid-lipofuscinoses-Batton; Charcot-Marie-Tooth Disease Type 2B; Charcot-Marie-Tooth Neuropathy Type 1B; Charcot-Marie-Tooth Neuropathy Type 2A2; Charge Syndrome; Cherubism; Choroideremia; Citrin Deficiency; Citrullinemia Type I; Coffin-Lowry Syndrome; Cohen Syndrome; Collagen 4A5; Common Variable Immune Deficiency; Congenital Adrenal Hyperplasia; Congenital Cataracts, Facial Dysmorphism, and Neuropathy; Congenital Disorder of Glycosylation Type 1a; Congenital Myasthenic Syndromes; Cornelia de Lange Syndrome; Cystic fibrosis; Cystinosis; Darier Disease; Desmin Storage Myopathy; DFNA2 Nonsyndromic Hearing Loss; Diamond-Blackfan Anemia; Double Cortex Syndrome; Duane Syndrome; Duchenne/Becker muscular dystrophy; Dysferlinopathy; Dyskeratosis Congenita; Early-Onset Familial Alzheimer Disease; Early-Onset Primary Dystonia (DYT1); Ehlers Danlos; Ehlers-Danlos Syndrome, Classic Type; Ehlers-Danlos Syndrome, Hypermobility Type; Ehlers-Danlos Syndrome, Kyphoscoliotic Form; Emery-Dreifuss Muscular Dystrophy X linked; Epidermolysis Bullosa Simplex; Fabry Disease; Facioscapulohumeral Muscular Dystrophy; Familial Dysautonomia (HSAN III); Familial Hyperinsulinism (FHI); Familial Hypertrophic Cardiomyopathy; Familial Transthyretin Amyloidosis; Fanconi Anemia; Fragile X; Friedreich Ataxia; FRMD7-Related Infantile Nystagmus; Fryns Syndrome; Galactosemia; Gaucher Disease; Glycine Encephalopathy; Glycogen Storage Disease Type VI; Hemophagocytic Lymphohistiocytosis; Hemophilia A; Hemophilia B; Hepatic Veno-Occlusive Disease with Immunodeficiency; Hereditary Hemorrhagic Telangiectasia; Hereditary Neuropathy with Liability to Pressure Palsies; Hereditary Nonpolyposis Colon Cancer; Hexosaminidase A Deficiency; HFE-Associated Hereditary Hemochromatosis; Holt-Oram Syndrome; Huntington Disease; Hydroxymethylbilane Synthase (HMBS) Deficiency; Hypophosphatasia; Inclusion Body Myopathy 2; Incontinentia Pigmenti; Juvenile Polyposis Syndrome; Kallmann Syndrome; Leber Congenital Amaurosis; Leber congenital amaurosis 10; Li-Fraumeni Syndrome; Limb-Girdle Muscular Dystrophy Type 2A Calpainopathy; LIS1-Associated Lissencephaly; Long QT Syndrome; Lowe Syndrome; Malignant Hyperthermia Susceptibility; Maple Syrup Urine Disease; MAPT-Related Disorders; McKusick-Kaufman Syndrome; MECP2-Rett Syndrome; Menkes; Metachromatic Leukodystrophy; Methylmalonic Acidemia; Mucolipidosis II; Multiple Endocrine Neoplasia Type 1; Multiple Endocrine Neoplasia Type 2; Myotonia Congenita; Myotonic Dystrophy Type 1; Myotonic Dystrophy Type 2; Nail-Patella Syndrome; Nemaline Myopathy; Neurofibromatosis 1; Neurofibromatosis 2; Noonan Syndrome; Ocular Albinism, X-Linked; Oculocutaneous Albinism Type 1; Oculocutaneous Albinism Type 2; Oculopharyngeal Muscular Dystrophy; Optic Atrophy Type 1; Ornithine Transcarbamylase Deficiency; Osteogenesis Imperfecta; Parkinson Disease; Pendred Syndrome; Peroxisome Biogenesis, Zellweger; Phenylketonuria; Polycystic Kidney Disease; Pompe Disease-GSD II; Primary Ciliary Dyskinesia; Retinitis Pigmentosa; Retinoblastoma; Saethre-Chotzen Syndrome; SCN9A-Related Inherited Erythromelalgia; SHOX-Related Haploinsufficiency; Sickle Cell Disease; Smith-Lemli-Opitz Syndrome; Smith-Magenis Syndrome; Sotos Syndrome; Spastic Paraplegia 3A; Spastic Paraplegia 7; Spastic Paraplegia 8; Spastic Paraplegia Type 1; Spastic Paraplegia Type 4; Spinal Muscular Atrophy; Spinocerebellar Ataxia 2; Spinocerebellar Ataxia 3; Spinocerebellar Ataxia 7; Spinocerebellar Ataxia Type 1; Stickler Syndrome; Thanatophoric Dysplasia; Thoracic Aortic Aneurysms and Aortic Dissections; Treacher Collins Syndrome; Trimethylaminuria; Tuberous Sclerosis Complex; Udd Distal Myopathy; Usher Syndrome type 1; Very Long Chain Acyl-Coenzyme A Dehydrogenase Deficiency; von Hippel-Lindau; Waardenburg Syndrome, Type 1; Werner Syndrome; Wilms Tumor; Wilson Disease; Wiskott-Aldrich; X-Linked Adrenal Hypoplasia Congenita; X-Linked Adrenoleukodystrophy; X-Linked Dystonia-Parkinsonism; X-linked Juvenile Retinoschisis; X-linked myotubular Myopathy; X-Linked SCIDS; and Zellweger Syndrome.

In one embodiment, the mutations associated with a congenital or inherited disease can include substitutions, insertions, inversions, point mutations, deletions, mismatches and translocations. In some embodiments, the mutations associated with an inherited or congenital disease includes copy number variation. In some embodiments, the disclosure relates generally to the selective amplification of at least one target sequence and the detection and/or identification of mutations associated with an inherited disease. In some embodiments, the mutations associated with a congenital or inherited disease can be located in one or more of the genes selected from the group consisting of ABCA4; ABCC8; ABCD1; ACADVL; ACTA2; ACTC; ACTC1; ACVRL1; ADA; AIPL1; AIRE; ALK1; ALPL; AMT; APC; APP; APTX; AR; ARL6; ARSA; ASL; ASPA; ASS; ASS1; ATL; ATM; ATP2A2; ATP7A; ATP7B; ATXN1; ATXN2; ATXN3; ATXN7; BBS6; BCKDHA; BCKDHB; BEST1; BMPR1A; BRCA1; BRCA2; BRIP1; BTD; BTK; C2orf25; CA4; CALR3; CAPN3; CAV3; CCDC39; CCDC40; CDH23; CEP290; CERKL; CFTR; CHAT; CHD7; CHEK2; CHM; CHRNA1; CHRNB1; CHRND; CHRNE; CLCN1; CNBP; CNGB1; COH1; COL11A1; COL11A2; COL1A1; COL1A2; COL2A1; COL3A1; COL4A5; COL5A1; COL5A2; COL7A1; COL9A1; CRB1; CRX; CTDP1; CTNS; CYP21A2; CYP27A1; DAX1; DBT; DCX; DES; DHCR7; DJ1; DKC1; DLD; DMD; DMPK; DNAAF1; DNAAF2; DNAH11; DNAH5; DNAI1;DNA12; DNAL1;DNM2; DOK7; DSC2; DSG2; DSP; DYSF; DYT1; EMD; ENG; EYA1; EYS; F8; F9; FANCA; FANCC; FANCF; FANCG;FANCJ; FANDC2; FBN1; FBXO7; FGFR1; FGFR3; FMO3; FMR1; FOXL2; FRG1; FRMD7; FSCN2; FXN; GAA; GALT; GBA; GBE1; GCSH; GDFS; GJB2; GJB3; GJB6; GLA; GLDC; GNE; GNPTAB; GPC3; GPR143; GUCY2D; HBA1; HBA2; HBB; HD; HERG; HEXA; HFE; HHF; HIBCH; HLA-B27; HMBS; HPLH1; HPRP3; HR; HTNB; HTT; IKBKAP; IKBKG; IL2RG; IMPDH1; ITGB4; JAG1; JPH3; KCNE1; KCNE2; KCNH2; KCNQ1; KCNQ4; KIAA0196; KLHL7; KRAS; KRT14; KRT5; L1CAM; LAMB3; LAMP2; LDB3; LMNA; LMX18; LRAT; LRRK2; MAPT; MC1R; MECP2; MED12; MEN1; MERTK; MFN2; MKKS; MLH1; MMAA; MMAB; MMACHC; MMADHC; MPZ; MSH2; MTM1; MTND5; MTTG; MTTI; MTTK; MTTL1; MTTQ; MUT; MYBPC3; MYH11; MYH6; MYH7; MYL2; MYL3; MYLK2; MYO7A; NDS; ND6; NEMO; NF1; NF2; NIPBL; NROB1; NR2E3; NRAS; NSD1; OCA2; OCRL; OPA1; OTC; PABPN1; PAFAH1B1; PAH; PARK2; PARK7; PARKIN; PAX3; PAX6; PCDH15; PEX1; PEX2; PEX10; PEX13; PEX14; PEX19; PEX26; PEX3; PEX5; PINK1; PKD1; PKD2; PKD3; PKHD1; PKP2; PLEC1; PLOD1; PMM2; PMP22; POLG; PPT1; PRCD; PRKAG2; PRNP; PROM1; PRPF3; PRPF8; PRPH2; PRPN; PSEN1; PSEN2; PTCH1; PTPN11; RAB7A; RAF1; RAI1; RAPSN; RB1; RDH12; RDS; RECQL3; RET; RHO; ROR2; RP1; RP2; RP9; RPE65; RPGR; RPGRIP1; RPL11; RPL35A; RPS10; RPS17; RPS19; RPS24; RPS26; RPS6KA3; RPS7; RPSL5; RS1; RSPH4A; RSPH9; RYR1; RYR2; SALL4; SCA3; SCNSA; SCN9A; SEMA4A; SERPINA1; SERPING1; SGCD; SH3BP2; SHOX; SIX1; SIX5; SLC25A13; SLC25A4; SLC26A4; SMAD4; SMN1; SNCA; SNRNP200; SOD1; SOS1; SOX9;SP110; SPAST; SPATA7; SPG3A; SPG4; SPG7; TAF1; TBXS; TCOF1; TGFBR1; TGFBR2; TNFRSC13C; TNNC1; TNNI3; TNNT1; TNNT2; TNXB; TOPORS; TOR1A; TP53; TPM1; TRNG; TRNI; TRNK; TRNL1; TRNQ; TSC1; TSC2; TTN; TTPA; TTR; TULP1; TWIST1; TXNDC3; TYR; USH1C; USH1H; USH2A; VCL; VHL; VPS1313; WAS; WRN; WT1; and ZNF9. [00156]Amplicon used as input to the verification methods. The pre-amplified nucleic acid used in the verification and sequencing methods of the invention may be obtained from many sources. The amplicon may be produced by PCR amplification of a size limited sample, including but not limited to the preamplification methods referred to here as the Ampliseq panels or assays. The amplicon may also be produced by bridge amplification such as may be used in Sequencing by Synthesis methods of sequencing. The amplicon may be produced via emulsion PCR while attached to a bead or surface. The amplicon may be produced by any form of amplification that can increase the amount of size limited sample to afford both sequencing via massively parallel processes as well as permitting the reserve of an aliquot of the preamplified sample to be used in the resequencing and verification methods of this invention.

Since the amplicon may be produced by many methods, the nature of its structure may be varied. The amplicon has at least a sequence of interest and a preceding sequence 5′ to the sequence of interest. This preceding sequence is introduced during the process used to preamplify the size limited sample. As such, the 5′preceding sequence itself may include two distinct regions; a process derived sequence portion including all or part of a 5′ portion of the 5′preceding sequence and a sequence specific region including all or a part of the 3′ portion of the 5′preceding sequence.

The 5′ process derived sequence region of the 5′ preceding sequence may have a wide variety of sequence types. The particular sequence depends on the process used for the preamplication. This 5′ process derived sequence region may be a “universal” primer sequence, a bar code sequence, a pull out sequence, and adaptor, a sequence used to immobilize the precursor sequence used to pre-amplify the limited sample, or some combination. The 5′ process derived sequence may be incorporated thru polymerase extension of a precursor species or by another type of incorporation, including but not limited to ligation. Each of these process derived sequences can be used to more selectively re-sequence, confirm or verify an initial sequencing analysis.

The 3′ sequence specific region of the 5′preceding sequence may be the portion of the primer that actually primes the specific extension of the primer species and thus provides for the expansion of the size limited sample. The 3′ portion can be used to focus the output of the preamp towards a preselected set of loci to be interrogated in the sequencing methods, for example, as in the AmpliSeq™ Cancer Hotspot Panel v.2.

Alternatively, the 5′ preceding sequence may have only a sequence specific region which includes the entire 5′ preceding sequence. For example, an amplicon produced by extension of a primer having a target-sequence specific oligonucleotide sequence would not have a process derived sequence portion, only a target-sequence specific oligonucleotide sequence for its entire length.

Chemically enhanced Primer. According to various embodiments of the present teachings, provided is a chemically-enhanced primer comprising an oligonucleotide sequence, a negatively charged moiety (NCM) and at least one nuclease-resistant linkage.

In some embodiments the at least one nuclease-resistant linkage includes but is not limited to at least one phosphorothioate linkage (PS) or at least one boronophosphate linkage. In other embodiments the nuclease-resistant linkage is not present in the chemically-enhanced primer. In yet other embodiments, a chemically-enhanced primer may comprise an oligonucleotide sequence, a negatively charged moiety (NCM), where the oligonucleotide inter-nucleotide linkages consist of phosphodiester inter-nucleotide linkages.

The primer can be used to prime a target nucleic acid in a sequencing reaction, herein referred to as a chemically-enhanced sequencing primer or for fragment analysis, herein referred to as a chemically-enhanced extension primer. The oligonucleotide sequence can be a universal primer or a gene specific nucleotide sequence. Examples of universal primers include but are not limited to M13 (P/N 402071 and 402072, Applied Biosystems), US1 (UNISEQ, PLoS Medicine 3(10)e431 (2006)), T7 (P/N 402126, but without dye, Applied Biosystems), SP6 (P/N 402128, but without dye, Applied Biosystems), and T3 (P/N 402127, but without dye, Applied Biosystems). The sequences for M13, T7, SP6 and T3 are shown in Table 1.

TABLE 1

| | |
|---|---|
| M13 Forward | 5′ TGT AAA ACG ACG GCC AGT 3′ |
| | (SEQ ID NO: 1) |
| M13 Reverse | 5′ CAG GAA ACA GCT ATG ACC 3′ |
| | (SEQ ID NO: 2) |
| T7 | 5′ TAA TAC GAC TCA CTA TAG GG 3′ |
| | (SEQ ID NO: 3) |
| SP6 | 5′ ATT TAG GTG ACA CTA TAG 3′ |
| | (SEQ ID NO: 4) |
| T3 | 5′ ATT AAC CCT CAC TAA AGG GA 3′ |
| | (SEQ ID NO: 5) |

The oligonucleotide sequence can also contain a dye-label such as a fluorescent label. In various embodiments of the present teachings the NCM can be located at the terminal 5′ end of the oligonucleotide sequence or within the oligonucleotide sequence. Examples of NCM include but are not limited to a phosphodiester moiety having a structure of the formula

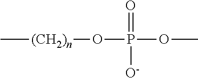

(which is introduced to the chemically-enhanced primer by reacting a phosphoramidite₇(available from Glen Research) with an appropriate reaction partner containing an oligonucleotide) referred to here as a (C)n spacer, wherein n can be from 1-12, the amino acids aspartic acid and glutamic acid as well as nucleotides and nucleotide analogs (dATP, dCTP, dGTP and dTTP). The NCM can contain only one negatively charged monomer or a plurality of negatively charged moieties, for example at least five, ten, 12, 15, 18, 20, 24 or more repeat units of the spacer, for example, (Cn)ₓ. where x is any integer between 1 and at least 11, at least 12, at least 15, at least 18, at least 20, at least 24 or 30 Cn spacers where “n” is 3 or 6, e.g., C3 spacers, C6 spacers or a combination of C3 and C6 spacers in a linear arrangement or a branched arrangement. The C3 and C6 spacers individually or in combination can also form a branched NCM by forming a doubler or a trebler such as, for example, (C3)₃-treb-M13 or [(C3)₂-treb]-treb-M13, where the NCM is represented by (C3)₃-treb or [(C3)₂-treb]-treb and M13 represents the oligonucleotide sequence, as would be known to one of skill in the art. The NCM can also contain a dye-label such as a fluorescent label. In various embodiments at least none, at least one, at least two or more phosphorothioate linkages can be at a terminal 3′end of the oligonucleotide sequence. The presence of at least one nuclease-resistant linkage provides resistance to digestion by 3′-5′ nucleases such as Exonuclease I (P/N M02935 New England Biolabs, Ipswich, Mass.), Exo III (P/N M02065, New England Biolabs, Ipswich, Mass.), Pfu (Promega, P/N M7741, Madison, Wis.), and DNA pol I (P/N M02095, New England Biolabs, Ipswich, Mass.). The resistance of the chemically-enhanced primer to nuclease digestion offers the advantage of eliminating a PCR clean-up step in the PCR to sequencing protocol. Removal of the extra non-nuclease resistant amplification primers left over from the PCR step can be accomplished in the sequencing reaction mixture. A brief exposure of the PCR amplification reaction to the nuclease within the sequencing reaction mixture degrades the non-nuclease resistant amplification primers followed by an inactivation of the nuclease. The chemically-enhanced primer remains available for the sequencing reaction while the non-nuclease resistant amplification primers and the nuclease have been removed and inactivated, respectively.

In some embodiments the chemically-enhanced primer has a structure of Formula I:

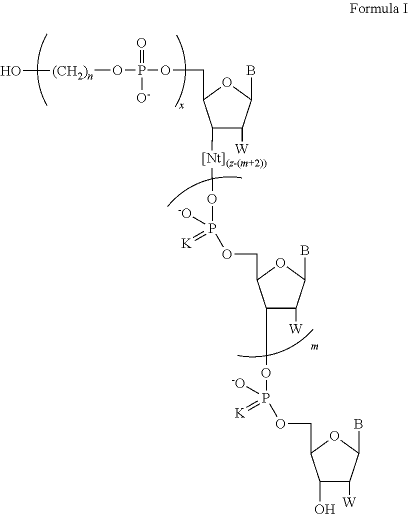

wherein B is a nucleobase; K is S or O; each n is independently an integer of 1 to 12; m is 0 or 1; × is an integer of 1 to about 50; z is an integer of 3 to about 100; W is OH, F, OMe, or H; and Nt is a moiety having a formula:

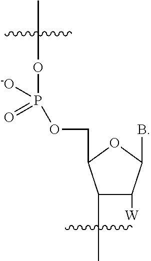

For chemically-enhanced primers having a structure of Formula I, OLIGO represents the portion of the chemically-enhanced primer of Formula I that comprises an oligonucleotide. Each nucleotide of the oligonucleotide comprises a nucleobase B portion and a ribose portion:

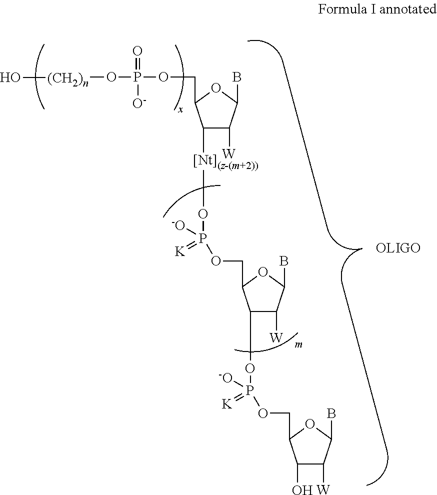

The chemically-enhanced primer of Formula I may comprise one or more B, wherein B is a naturally occurring nucleobase. In other embodiments, the chemically-enhanced primer of Formula I may comprise one or more B, wherein B is a nucleobase analog.

The chemically-enhanced primer of Formula I may have only one phosphothiorate linkage, wherein m is 0, having a structure of Formula I-A:

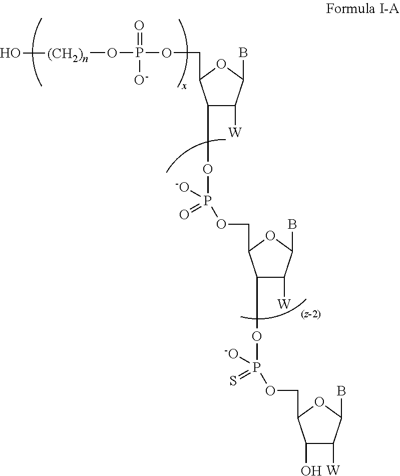

The chemically-enhanced primer of Formula I may be labeled with a dye, including dyes that are fluorescent. The chemically-enhanced primer of Formula I may include one or more B labeled with a dye, and is represented as B^{f}. In some embodiments, the 3′ terminal nucleotide of the chemically-enhanced primer has a fluorescently labeled B. The chemically-enhanced primer may contain a 3′ fluorescently labeled terminal nucleotide wherein the B of the 3′ terminal nucleotide is a nucleobase analog. Alternatively, the chemically-enhanced primer may contain a 5′ terminal nucleotide having a fluorescently labeled B, which can be represented as B^{f}. In some embodiments, wherein the chemically-enhanced primer contains a 5′ terminal nucleotide containing the fluorescently labeled nucleobase, B^{f}, the labeled nucleobase is a nucleobase analog. In other embodiments, the chemically-enhanced primer may contain a fluorescently labeled NCM attached directly or indirectly to one of a plurality of NCMs and/or a linker moiety to the 5′ terminal nucleotide of the primer. Additionally, the chemically-enhanced primer of Formula I may be fluorescently labeled on the nucleobase of a nucleotide located at an internal position of the oligonucleotide, and the internal fluorescently labeled nucleotide may be selected to be at any position of the non-terminal portion of the oligonucleotide.

When the chemically-enhanced primer of Formula I contains a fluorescent label, the chemically-enhanced primer may have a structure of one of the following formulae:

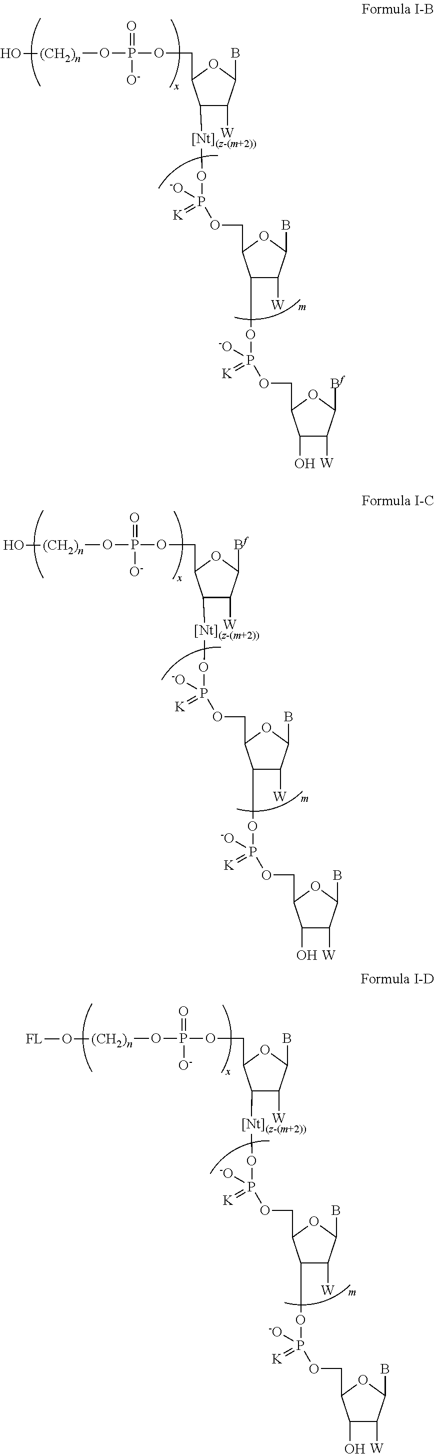

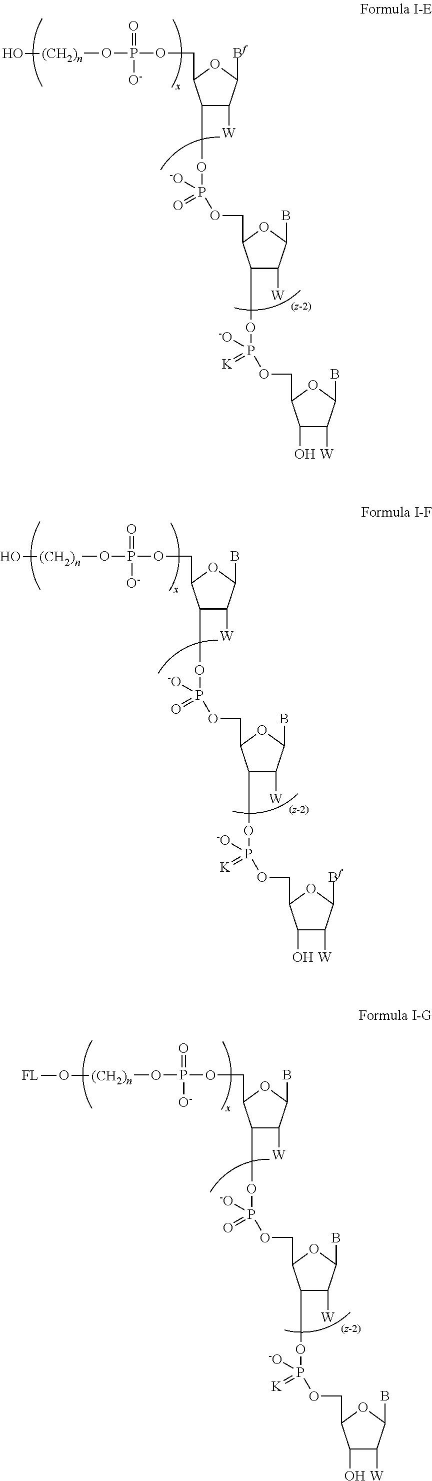

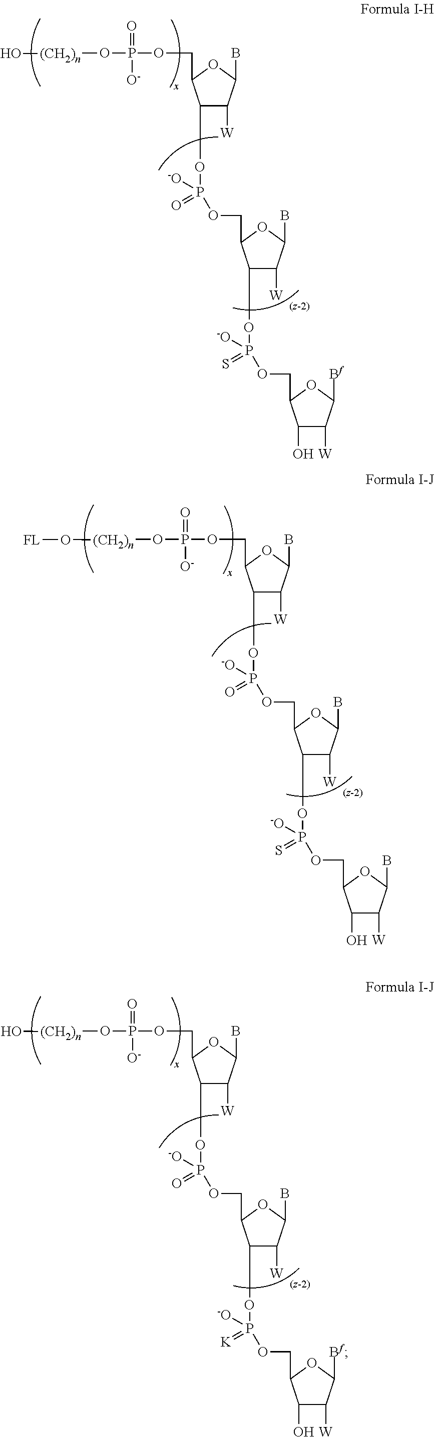

wherein FL is a dye label and B^{f} is a dye labeled nucleobase . Fl and B^{f} may each represent a fluorescent dye label.

For the chemically-enhanced primer of Formula I, each n can independently be an integer of 1 to 12. In some embodiments, n is 1, 2, 3, 4, 5, 6, 7, 8, or 9. In some embodiments, n is 3. In other embodiments, n is 4. Alternatively, n may be 6. In some embodiments of the chemically enhanced primer of Formula I, when x is greater than 2, a first instance of n is selected to be 3 and a second instance of n is selected to be 6. In further embodiments of the chemically-enhanced primers of Formula I, when x is greater than 2, more than one instance of n is selected to be 3, and more than one instance of n is selected to be 6. In yet other embodiments, when x is greater than 5, a plurality of n is selected to be 3, and a second plurality of n is selected to be 6.

The chemically-enhanced primer of Formula I may have m=1 or m=0. In some embodiments the chemically-enhanced primer of Formula I has m=0.

The chemically-enhanced primer of Formula I may have x, wherein x is an integer of 1 to about 50. In some of the embodiments of the chemically-enhanced primer of Formula I, x is 1, 2, 3, 4, 5, 6, 7, 8, 9, or 10. In other embodiments, x is 10, 15, 18, 20 or 24. In some embodiments, x is 5, 8, 9, 10 or 15. In other embodiments, x is 11, 12, 13, 14, 17 or 20. In other embodiments, x is 30. In further embodiments, x is at least 5, at least 6, at least 8, at least 9, at least 10, at least 15 at least 18, at least 20, or at least 24. In some embodiments, x is 15. In yet other embodiments, x is 8 or 9.

In some embodiments, the chemically-enhanced primers comprise a second plurality y of

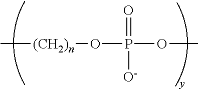

moieties, wherein y is an integer of 1-20. In some embodiments, when a first plurality x of n has a value of a first integer, then a second plurality y of n is an integer of 1 to 20. In some embodiments, the chemically-enhanced primer may have a first plurality of n wherein n is 3 and x is 15, and a second plurality of n wherein n is 6 and x is 5. All combinations of n, x and y are contemplated for use in the chemically-enhanced primers of Formula I.

In the chemically-enhanced primer of Formula I, z is an integer of 3 to about 100. In some embodiments, z is an integer of 5 to 50, 5 to 40, or 5 to about 30. In other embodiments, z is an integer of 5 to 25, or 5 to 20.

In some of the embodiments of the chemically-enhanced primer of Formula I, K is S. In other embodiments, K is O.

In some embodiments of the chemically-enhanced primer of Formula I, W is H or OH.

The chemically-enhanced primer of Formula I, I-B, I-C, I-E, I-F, or I-G, may have any combination of B, B^{f}, FL, K, m, n, W, x, and z of the ranges and selections disclosed above.

The chemically-enhanced primer of Formula I-D may have any combination of B, FL, K, m, n, W, x, and z of the ranges and selections disclosed above.

The chemically-enhanced primer of Formula I-A, I-H, I-J or I-K, may have any combination of B, B^{f}, FL, K, m, n, W, x, and z of the ranges and selections disclosed above.

In other embodiments, the chemically-enhanced primer is a compound having a structure of Formula II:

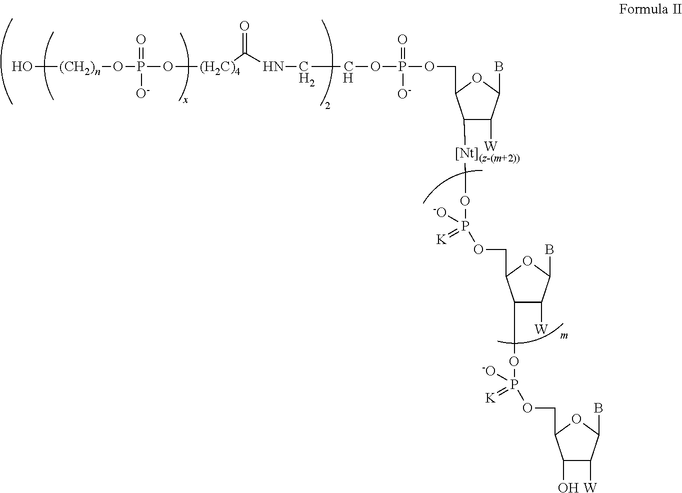

wherein B is a nucleobase; K is S or O; each n is independently an integer of 1 to 12; m is 0 or 1; × is an integer of 1 to about 50; z is an integer of 3 to about 100; W is OH, F, OMe, or H; and Nt is a moiety having a formula:

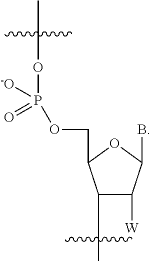

The chemically-enhanced primer of Formula II may be referred to as a doubler, and represents a branched arrangement of NCM moieties.

The chemically-enhanced primer of Formula II may comprise one or more B, wherein B is a naturally occurring nucleobase. In other embodiments, the chemically-enhanced primer of Formula II may comprise one or more B, wherein B is a nucleobase analog.

The chemically-enhanced primer of Formula II may have only one phosphothiorate linkage, wherein m is 0.

In some embodiments, the chemically-enhanced primer of Formula II may be labeled with a dye, including dyes that are fluorescent. The chemically-enhanced primer of Formula II may include one or more B labeled with a dye, and is represented as B^{f}. In some embodiments, the 3′ terminal nucleotide of the chemically-enhanced primer has a fluorescently labeled B. The chemically-enhanced primer may contain a 3′ fluorescently labeled terminal nucleotide wherein the B of the 3′ terminal nucleotide is a nucleobase analog. Alternatively, the chemically-enhanced primer may contain a 5′ terminal nucleotide having a fluorescently labeled B, which can be represented as B^{f}. In some embodiments, wherein the chemically-enhanced primer contains a 5′ terminal nucleotide containing the fluorescently labeled nucleobase, B^{f}, the labeled nucleobase is a nucleobase analog. In other embodiments, the chemically-enhanced primer may contain a fluorescently labeled NCM attached directly or indirectly to one of a plurality of NCMs and/or a linker moiety to the 5′ terminal nucleotide of the primer. Additionally, the chemically-enhanced primer of Formula II may be fluorescently labeled on the nucleobase of a nucleotide located at an internal position of the oligonucleotide, and the internal fluorescently labeled nucleotide may be selected to be at any position of the non-terminal portion of the oligonucleotide.

For the chemically-enhanced primer of Formula II, n can be an integer of 1 to 9. In some embodiments, n is 1, 2, 3, 4, 5, 6, 7, 8, or 9. In some embodiments, n is 3. In other embodiments, n is 4. Alternatively, n may be 6. . In some embodiments of the chemically-enhanced primer of Formula II, when x is greater than 2, a first instance of n is selected to be 3 and a second instance of n is selected to be 6. In further embodiments of the chemically-enhanced primers of Formula II, when x is greater than 2, more than one instance of n is selected to be 3, and more than one instance of n is selected to be 6. In yet other embodiments, when x is greater than 5, a plurality of n is selected to be 3, and a second plurality of n is selected to be 6.

The chemically-enhanced primer of Formula II may have m=1 or m=0. In some embodiments the chemically-enhanced primer of Formula II has m=0.

The chemically-enhanced primer of Formula II may have x wherein x is an integer of 1 to about 50. In some of the embodiments of the chemically-enhanced primer of Formula II, x is 1, 2, 3, 4, 5, 6, 7, 8, 9, or 10. In other embodiments, x is 10, 15, 18, 20 or 24. In some embodiments, x is 5, 8, 9, 10 or 15. In other embodiments, x is 11, 12, 13, 14, 17 or 20. In other embodiments, x is 30. In further embodiments, x is at least 5, at least 6, at least 8, at least 9, at least 10, at least 15 at least 18, at least 20, or at least 24. In some embodiments, x is 15. In yet other embodiments, x is 8 or 9.

In some embodiments, the chemically-enhanced primers comprise a second plurality y of

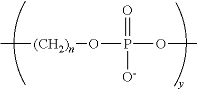

moieties, wherein y is an integer of 1-20. In some embodiments, when a first plurality x of n has a value of a first integer, then a second plurality y of n is an integer of 1 to 20. In some embodiments, the chemically-enhanced primer may have a first plurality of n wherein n is 3 and x is 15, and a second plurality of n wherein n is 6 and x is 5. All combinations of n, x and y are contemplated for use in the chemically-enhanced primers of Formula II.

In the chemically-enhanced primer of Formula II, z is an integer of 3 to about 100. In some embodiments, z is an integer of 5 to 50, 5 to 40, or 5 to about 30. In other embodiments, z is an integer of 5 to 25, or 5 to 20. In some of the embodiments of the chemically-enhanced primer of Formula II, K is S. In other embodiments, K is O. In some embodiments of the chemically-enhanced primer of Formula II, W is H or OH. The chemically-enhanced primer of Formula II may have any combination of B, K, m, n, W, x, and z of the ranges and selections disclosed above.

In yet other embodiments, the chemically-enhanced primer is a compound having a structure of the Formula III:

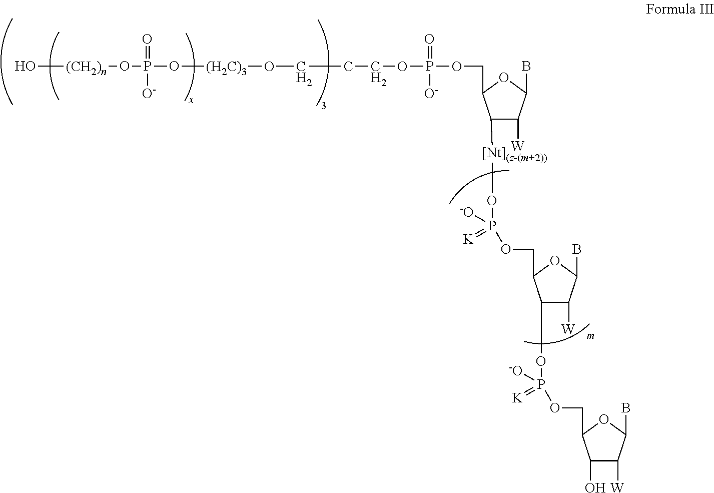

wherein B is a nucleobase; K is S or O; each n is independently an integer of 1 to 12; m is 0 or 1; x is an integer of 1 to about 50; z is an integer of 3 to about 100; W is OH, F, OMe, or H; and Nt is a moiety having a formula:

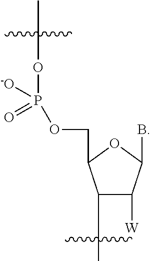

The chemically-enhanced primer of Formula III may be referred to as a trebler and represents a branched arrangement of NCM moieties.

The chemically-enhanced primer of Formula III may comprise one or more B, wherein B is a naturally occurring nucleobase. In other embodiments, the chemically-enhanced primer of Formula III may comprise one or more B, wherein B is a nucleobase analog.

The chemically-enhanced primer of Formula III may have only one phosphothiorate linkage, wherein m is 0.

The chemically-enhanced primer of Formula III may be labeled with a dye, including dyes that are fluorescent. The chemically-enhanced primer of Formula III may include one or more B labeled with a dye, and is represented as B^{f}. In some embodiments, the 3′ terminal nucleotide of the chemically-enhanced primer has a fluorescently labeled B. The chemically-enhanced primer may contain a 3′ fluorescently labeled terminal nucleotide wherein the B of the 3′ terminal nucleotide is a nucleobase analog. Alternatively, the chemically-enhanced primer may contain a 5′ terminal nucleotide having a fluorescently labeled B, which can be represented as B^{f}. In some embodiments, wherein the chemically-enhanced primer contains a 5′ terminal nucleotide containing the fluorescently labeled nucleobase, B^{f}, the labeled nucleobase is a nucleobase analog. In other embodiments, the chemically-enhanced primer may contain a fluorescently labeled NCM attached directly or indirectly to one of a plurality of NCMs and/or a linker moiety to the 5′ terminal nucleotide of the primer. Additionally, the chemically-enhanced primer of Formula II may be fluorescently labeled on the nucleobase of a nucleotide located at an internal position of the oligonucleotide, and the internal fluorescently labeled nucleotide may be selected to be at any position of the non-terminal portion of the oligonucleotide.

For the chemically-enhanced primer of Formula III, n can be an integer of 1 to 9. In some embodiments, n is 1, 2, 3, 4, 5, 6, 7, 8, or 9. In some embodiments, n is 3. In other embodiments, n is 4. Alternatively, n may be 6. In some embodiments of the chemically-enhanced primer of Formula III, when x is greater than 2, a first instance of n is selected to be 3 and a second instance of n is selected to be 6. In further embodiments of the chemically-enhanced primers of Formula III, when x is greater than 2, more than one instance of n is selected to be 3, and more than one instance of n is selected to be 6. In yet other embodiments, when x is greater than 5, a plurality of n is selected to be 3, and a second plurality of n is selected to be 6.

The chemically-enhanced primer of Formula III may have m=1 or m=0. In some embodiments the chemically-enhanced primer of Formula III has m=0.

The chemically-enhanced primer of Formula III may have x wherein x is an integer of 1 to about 30. In some of the embodiments of the chemically-enhanced primer of Formula II, x is 1, 2, 3, 4, 5, 6, 7, 8, 9, or 10. In other embodiments, x is 10, 15, 18, 20 or 24. In some embodiments, x is 5, 8, 9, 10 or 15. In other embodiments, x is 11, 12, 13, 14, 17 or 20. In other embodiments, x is 30. In further embodiments, x is at least 5, at least 6, at least 8, at least 9, at least 10, at least 15 at least 18, at least 20, or at least 24. In some embodiments, x is 15. In yet other embodiments, x is 8 or 9.

In the chemically-enhanced primer of Formula III, z is an integer of 3 to about 100. In some embodiments, z is an integer of 5 to 50, 5 to 40, or 5 to about 30. In other embodiments, z is an integer of 5 to 25, or 5 to 20.

In some embodiments, the chemically-enhanced primers comprise a second plurality y of

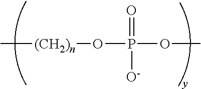

moieties, wherein y is an integer of 1-20. In some embodiments, when a first plurality x of n has a value of a first integer, then a second plurality y of n is an integer of 1 to 20. In some embodiments, the chemically-enhanced primer may have a first plurality of n wherein n is 3 and x is 15, and a second plurality of n wherein n is 6 and x is 5. All combinations of n, x and y are contemplated for use in the chemically-enhanced primers of Formula III.

In some of the embodiments of the chemically-enhanced primer of Formula III, K is S. In other embodiments, K is O. In some embodiments of the chemically-enhanced primer of Formula III, W is H or OH. The chemically-enhanced primer of Formula III may have any combination of B, K, m, n, W, x, and z of the ranges and selections disclosed above.Other embodiments of the chemically-enhanced primer are represented by Formula IV:

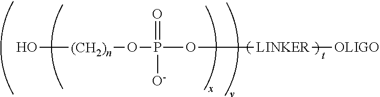

### Formula IV

wherein each instance of n is independently an integer of 1 to 12; × is an integer of 1 to 50;

v is an integer of 1 to 9; t is 0 or 1; LINKER comprises 3-100 atoms;

OLIGO has a structure of the following formula:

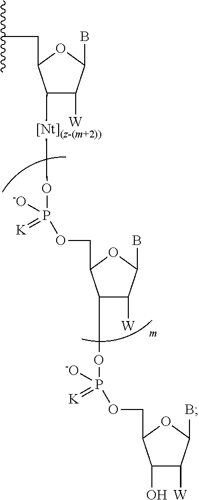

wherein B is a nucleobase; K is S or O; m is 0 or 1; z is an integer of 3 to about 100; W is OH, F, OMe, or H; and Nt is a moiety having a formula:

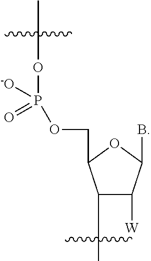

The chemically-enhanced primer of Formula IV, may comprise one or more B wherein B is a naturally occurring nucleobase. In other embodiments, the chemically-enhanced primer of Formula IV may comprise one or more B, wherein B is a nucleobase analog.

The chemically-enhanced primer of Formula IV may be labeled with a dye, including dyes that are fluorescent. The chemically-enhanced primer having a formula of (Cn)ₓ-OLIGO may include one or more B labeled with a dye, and is represented as B^{f}. In some embodiments, when the chemically-enhanced primer has at least one B labeled with a dye, the B may be a nucleobase analog. In some embodiments, the 3′ terminal nucleotide of the chemically-enhanced primer has a fluorescently labeled B, which can be represented as B^{f}. Alternatively, the chemically-enhanced primer may contain a 5′ terminal nucleotide having a fluorescently labeled B, which can be represented as B^{f}. Additionally, the chemically-enhanced primer having a formula of (Cn)ₓ-OLIGO, may be fluorescently labeled on the nucleobase of a nucleotide located at an internal position of the oligonucleotide, and the internal fluorescently labeled nucleotide may be selected to be at any position of the non-terminal portion of the oligonucleotide. In other embodiments, the chemically-enhanced primer may contain a fluorescently labeled NCM attached directly or indirectly to one of a plurality of NCMs and/or to a NCM linker moiety forming a covalent attachment to the 5′ terminal nucleotide of the primer.

LINKER is an NCM linker and may comprise 3-100 atoms and include ether, amide, phosphodiester, and ester moieties to form a covalent linkage between the NCM and the oligonucleotide. LINKER may be attached to the 5′ carbon of the ribose of the nucleotide at the 5′ terminus of the oligonucleotide. In some embodiments, LINKER is present. In other embodiments the NCM phosphodiester moiety or moieties are directly attached to OLIGO.

For the chemically-enhanced primer of Formula IV, v can be an integer of 1 to 9. In some embodiments, v is 1. In other embodiments, v is 2. In yet other embodiments, v is 3.

For the chemically-enhanced primer of Formula IV, n can be an integer of 1 to 12. In some embodiments, n is 1, 2, 3, 4, 5, 6, 7, 8, or 9. In other embodiments, n is an integer of 1 to 9. In some embodiments, n is 3. In other embodiments, n is 4. Alternatively, n may be 6The chemically-enhanced primer of Formula IV has x , wherein x is an integer of 1 to about 30. In some of the embodiments of the chemically-enhanced primer of Formula IV, x is 1, 2, 3, 4, 5, 6, 7, 8, 9, or 10. In other embodiments, x is 10, 15, 18, 20 or 24. In some embodiments, x is 5, 8, 9, 10 or 15. In other embodiments, x is 11, 12, 13, 14, 17 or 20. In further embodiments, x is at least 5, at least 6, at least 8, at least 9, at least 10, at least 15 at least 18, at least 20, or at least 24. In some embodiments, x is 15. In yet other embodiments, x is 8 or 9.

In some embodiments of the chemically-enhanced primer of Formula IV, when x is greater than 2, a first instance of n is selected to be 3 and a second instance of n is selected to be 6. In further embodiments of the chemically-enhanced primers of Formula IV, when x is greater than 2, more than one instance of n is selected to be 3, and more than one instance of n is selected to be 6. In yet other embodiments, when x is greater than 5, a plurality of n is selected to be 3, and a second plurality of n is selected to be 6.

In some embodiments, the chemically-enhanced primers comprise a second plurality y of

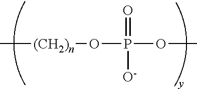

moieties, wherein y is an integer of 1- 20. In some embodiments, when a first plurality x of n has a value of a first integer, then a second plurality y of n is an integer of 1 to 20. In some embodiments, the chemically-enhanced primer may have a first plurality of n wherein n is 3 and x is 15, and a second plurality of n wherein n is 6 and x is 5. All combinations of n, x and y are contemplated for use in the chemically-enhanced primers of Formula IV.

The chemically-enhanced primer having a formula of Formula IV has m=1 or m=0. In some embodiments the chemically-enhanced primer of Formula IV has m=0. The chemically-enhanced primer having a formula of Formula IV has z , wherein z is an integer of 3 to about 100. In some embodiments, z is an integer of 5 to 50, 5 to about 40, or 5 to about 30. In other embodiments, z is an integer of 5 to 25, or 5 to 20. In some of the embodiments of the chemically-enhanced primer of Formula IV, K is S. In other embodiments, K is O. In some embodiments of the chemically-enhanced primer of Formula IV, W is H or OH. The chemically-enhanced primer of Formula IV, may have any combination of B, n, t, v, x, m, y, z, K or W of the ranges and selections disclosed above.

In some embodiments, the chemically-enhanced primer of Formula IV is a chemically-enhanced primer (Cn)ₓ-OLIGO , wherein (Cn)ₓ has a structure of the following formula:

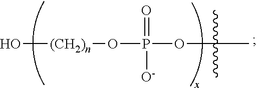

wherein each instance of n is independently an integer of 1 to 12; and x is an integer of 1 to about 30; and OLIGO has a structure of the following formula:

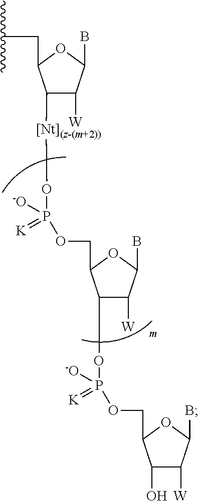

wherein B, K, m, z, y, Nt, and W are as defined above for Formula IV.

For the chemically-enhanced primer having a formula of (Cn)ₓ-OLIGO, v is 1, t is 0, no LINKER is present, and the chain of NCM moieties are attached to OLIGO directly. [00212]The chemically-enhanced primer having a formula of (Cn)ₓ-OLIGO, may have any combination of B, n, x, m, z, K or W of the ranges and selections disclosed above for Formula IV.

In yet other embodiments, the chemically-enhanced primer is represented by the following formulae:

(Cn)ₓ-OLIGO* , wherein (Cn)ₓ has a structure of the following formula:

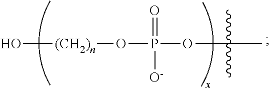

wherein each instance of n is independently an integer of 1 to 12; and x is an integer of 1 to about 30; OLIGO* has a structure of the following formula:

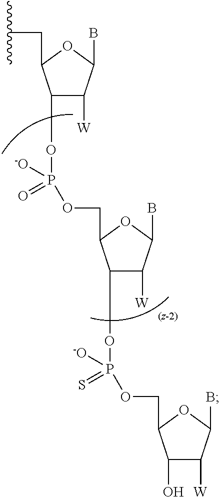

wherein B, K, m, z, y, Nt, and W are as defined above for Formula IV.

For the chemically-enhanced primer having a formula of (Cn)ₓ-OLIGO*, v is 1, t is 0, no LINKER is present, and the chain of NCM moieties are attached to OLIGO* directly. [00215]The chemically-enhanced primer having a formula of (Cn)ₓ-OLIGO*, may have any combination of B, n, x, m, z, or W of the ranges and selections disclosed above for Formula IV.

Chemically-enhanced primers having a formula of (Cn)ₓ Formula VI-Al include , but are not limited to:
(Cn)ₓ-US1, where n is 1 to 9 and x is 1 to about 30. In some embodiments, (Cn)ₓ-US1 is (C3)₁-US1, (C3)₂-US1, (C3)₃-US1, (C3)₄-US1, (C3)₅-US1, (C3)₆-US1,(C3)₇-US1, (C3)₈-US1, (C3)₉-US1, (C3)₁₀-US1, (C3)₁₁-US1, (C3)₁₂-US1, (C3)₁₃-US1, (C3)₁₄-US1 , (C3)₁₅-US1, (C3)₁₆-US1, (C3)₁₇-US1, (C3)₁₈-US1, (C3)₂₁-US1, (C3)₂₄-US1, (C3)₂₇-US1, or (C3)₃₀ -US1. In some embodiments, (Cn)ₓ-US1is a forward primer and may have any x as described above. In other embodiments, (Cn)ₓ-US1 is a reverse primer and may have any x as described above.
(Cn)ₓ-M13-forward, where n is 1 to 9 and x is 1 to about 30. In some embodiments, (Cn)ₓ-M13-forward is (C3)₁-M13-forward, (C3)₂-M13-forward, (C3)₃-M13-forward, (C3)₄-M13-forward, (C3)₅-M13-forward, (C3)₆-M13-forward, (C3)₇-M13-forward, (C3)₈-M13-forward , (C3)₉-M13-forward, (C3)₁₀-M13-forward, (C3)₁₁-M13-forward, (C3)₁₂-M13-forward, (C3)₁₃-M13-forward, (C3)₁₄-M13-forward, (C3)₁₅-M13-forward, (C3)₁₆-M13-forward, (C3)₁₇-M13-forward, (C3)₁₈-M13-forward, (C3)₂₁-M13-forward, (C3)₂₄-M13-forward, (C3)₂₇-M13-forward, or (C3)₃₀ -M13-forward.
(Cn)ₓ-M13-reverse, where n is 1 to 9 and x is 1 to about 30. In some embodiments, (Cn)ₓ-M13-reverse is (C3)₁-M13-reverse, (C3)₂-M13-reverse, (C3)₃-M13-reverse, (C3)₄-M13-reverse, (C3)₅-M13-reverse, (C3)₆-M13-reverse, (C3),- M13-reverse, (C3)₈-M13-reverse, (C3)₉-M13-reverse, (C3)₁₀-M13-reverse, (C3)₁₁-M13-reverse, (C3)₁₂-M13-reverse, (C3)₁₃-M13-reverse, (C3)₁₄-M13-reverse, (C3)₁₅-M13-reverse, (C3)₁₆-M13-reverse, (C3)₁₇-M13-reverse, (C3)₁₈-M13-reverse, (C3)₂₁-M13-reverse, (C3)₂₄-M13-reverse, (C3)₂₇-M13-reverse, or (C3)₃₀ -M13-reverse.
(Cn)ₓ-T7, where n is 1 to 9 and x is 1 to about 30. In some embodiments, (Cn)ₓ-T7 is (C3)₁-T7, (C3)₂-T7, (C3)₃-T7 (C3)₄-T7, (C3)₅-T7, (C3)₆-T7, (C3)₇-T7, (C3)₈-T7, (C3)₉- T7, (C3)₁₀-T7, (C3)₁₁-T7, (C3)₁₂-T7, (C3)₁₃-T7, (C3)₁₄-T7, (C3)₁₅-T7, (C3)₁₆-T7, (C3)₁₇-T7, (C3)₁₈-T7, (C3)₂₁-T7, (C3)₂₄-T7, (C3)₂₇-T7, or (C3)₃₀ -T7. In some embodiments, (Cn)ₓ-T7_is a forward primer and may have any x as described above. In other embodiments, (Cn)ₓ-T7 is a reverse primer and may have any x as described above.
(Cn)ₓ-SP6, where n is 1 to 9 and x is 1 to about 30. In some embodiments, (Cn)ₓ-SP6 is (C3)₁-SP6, (C3)₂-SP6,(C3)₃-SP6,(C3)₄-SP6, (C3)₅-SP6, (C3)₆-SP6,(C3)₇-SP6, (C3)₈-SP6, (C3)₉-SP6,(C3)₁₀-SP6, (C3)₁₁-SP6, (C3)₁₂-SP6, (C3)₁₃-SP6, (C3)₁₄-SP6 , (C3)₁₅-SP6, (C3)₁₆-SP6, (C3)₁₇-SP6, (C3)₁₈-SP6, (C3)₂₁-SP6, (C3)₂₄-SP6, (C3)₂₇-SP6, or (C3)₃₀ -SP6. In some embodiments, (Cn)ₓ-SP6 is a forward primer and may have any x as described above. In other embodiments, (Cn)ₓ-SP6 is a reverse primer and may have any x as described above.
(Cn)ₓ-T3, where n is 1 to 9 and x is 1 to about 30. In some embodiments, (Cn)ₓ T3 is (C3)₁-T3, (C3)₂-T3,(C3)₃-T3,(C3)₄-T3, (C3)₅-T3, (C3)₆-T3,(C3)₇-T3, (C3)₈-T3, (C3)₉- T3,(C3)₁₀-T3, (C3)₁₁-T3, (C3)₁₂-T3, (C3)₁₃-T3, (C3)₁₄-T3 , (C3)₁₅-T3, (C3)₁₆-T3, (C3)₁₇-T3, (C3)₁₈-T3, (C3)₂₁-T3, (C3)₂₄-T3, (C3)₂₇-T3, or (C3)₃₀ -T3. In some embodiments, (Cn)ₓ-T3 is a forward primer and may have any x as described above. In other embodiments, (Cn)ₓ-T3 is a reverse primer and may have any x as described above.
(Cn)ₓ-GSO, where n is 1 to 9, × is 1 to about 30, and GSO is a gene specific oligonucleotide sequence, wherein the gene specific oligonucleotide comprises 50 or fewer nucleotides. In some embodiments, (Cn)ₓ GSO is (C3)₁-GSO, (C3)₂-GSO, (C3)₃-GSO, (C3)₄-GSO, (C3)₅-GSO, (C3)₆-GSO, (C3)₇-GSO, (C3)₈-GSO, (C3)₉-GSO, (C3)₁₀-GSO, (C3)₁₁-GSO, (C3)₁₂-GSO, (C3)₁₃-GSO, (C3)₁₄-GSO, (C3)₁₅-GSO, (C3)₁₆-GSO, (C3)₁₇-GSO, (C3)₁₈-GSO, (C3)₂₁-GSO, (C3)₂₄-GSO, (C3)₂₇-GSO, or (C3)₃₀ -GSO. In some embodiments, (Cn)ₓ-GSO is a forward primer and may have any x as described above. In other embodiments, (Cn)ₓ-GSO is a reverse primer and may have any x as described above.

Chemically-enhanced primers having a formula of (Cn)ₓ-OLIGO* include, but are not limited to:
(Cn)ₓ-US1*, where n is 1 to 9 and x is 1 to about 30. In some embodiments, (Cn)ₓ-US1* is (C3)₁-US1*, (C3)₂-US1*, (C3)₃-US1*, (C3)₄-US1*, (C3)₅-US1*, (C3)₆-US1*, (C3)₇-US1*, (C3)₈-US1*, (C³)₉-US1*, (C3)₁₀-US1*, (C3)₁₁-US1*, (C3)₁₂-US1*, (C3)₁₃-US1*, (C3)₁₄-US1v, (C3)₁₅-US1*, (C3)₁₆-US1*, (C3)₁₇-US1*, (C3)₁₈-US1*, (C3)₂₁-US1*, (C3)₂₄US1*, (C3)₂₇-US1*, or (C3)₃₀-US1*. In some embodiments, (Cn)ₓ-US1* is a forward primer and may have any x as described above. In other embodiments, (Cn)ₓ-US1* is a reverse primer and may have any x as described above.
(Cn)ₓ-M13*-forward, where n is 1 to 9 and x is 1 to about 30. In some embodiments, (Cn)ₓ-M13*-forward is (C3)₁-M13*-forward, (C3)₂-M13*-forward, (C3)₃-M13*-forward, (C3)₄-M13*-forward, (C3)₅-M13*-forward, (C3)₆-M13*-forward, (C3),- M13*-forward, (C3)₈-M13*-forward, (C3)₉-M13*-forward, (C3)₁₀-M13*-forward, (C3)₁₁-M13*-forward, (C3)₁₂-M13*-forward, (C3)₁₃-M13*-forward, (C3)₁₄-M13*-forward, (C3)₁₅-M13*-forward, (C3)₁₆-M13*-forward, (C3)₁₇-M13*-forward, (C3)₁₈-M13*-forward, (C3)₂₁-M13*-forward, (C3)₂₄-M13*-forward, (C3)₂₇-M13*-forward, or (C3)₃₀ -M13*-forward.
(Cn)ₓ-M13*-reverse, where n is 1 to 9 and x is 1 to about 30. In some embodiments, (Cn)ₓ-M13*-reverse is (C3)₁-M13*-reverse, (C3)₂-M13*-reverse, (C3)₃-M13*-reverse, (C3)₄-M13*-reverse, (C3)₅-M13*-reverse, (C3)₆-M13*-reverse, (C3),- M13*-reverse, (C3)₈-M13*-reverse, (C3)₉-M13*-reverse, (C3)₁₀-M13*-reverse, (C3)₁₁-M13*-reverse, (C3)₁₂-M13*-reverse, (C3)₁₃-M13*-reverse, (C3)₁₄-M13*-reverse, (C3)₁₅-M13*-reverse, (C3)₁₆-M13*-reverse, (C3)₁₇-M13*-reverse, (C3)₁₈-M13*-reverse, (C3)₂₁-M13*-reverse, (C3)₂₄-M13*-reverse, (C3)₂₇-M13*-reverse, or (C3)₃₀ -M13*-reverse.
(Cn)ₓ-T7*, where n is 1 to 9 and x is 1 to about 30. In some embodiments, (Cn)ₓ-T7* is (C3)₁-T7*, (C3)₂-T7*, (C3)₃-T7*, (C3)₄-T7*, (C3)₅-T7*, (C3)₆-T7*, (C3)₇-T7*, (C3)₈-T7*, (C3)₉-T7^{*}, (C3)₁₀-T7*, (C3)₁₁-T7*, (C3)₁₂-T7*, (C3)₁₃-T7*, (C3)₁₄-T7*, (C3)₁₅-T7*, (C3)₁₆-T7*, (C3)₁₇-T7*, (C3)₁₈-T7*, (C3)₂₁-T7*, (C3)₂₄-T7*, (C3)₂₇-T7*, or (C3)₃₀ -T7*. In some embodiments, (Cn)ₓ-T7* is a forward primer and may have any x as described above. In other embodiments, (Cn)ₓ-T7* is a reverse primer and may have any x as described above.
(Cn)ₓ-SP6*, where n is 1 to 9 and x is 1 to about 30. In some embodiments, (Cn)ₓ-SP6* is (C3)₁-SP6*, (C3)₂-SP6^{*}, (C3)₃-SP6^{*}, (C3)₄-SP6*, (C3)₅-SP6*, (C3)₆-SP6*, (C3)₇-SP6*, (C3)₈-SP6*, (C³)₉-SP6*, (C3)₁₀-SP6*, (C3)₁₁-SP6*, (C3)₁₂-SP6*, (C3)₁₃-SP6*, (C3)₁₄-SP6*, (C3)₁₅-SP6*, (C3)₁₆-SP6*, (C3)₁₇-SP6*, (C3)₁₈-SP6*, (C3)₂₁-SP6*, (C3)₂₄-SP6*, (C3)₂₇-SP6*, or (C3)₃₀-SP6*. In some embodiments, (Cn)ₓ-SP6*_is a forward primer and may have any x as described above. In other embodiments, (Cn)ₓ-SP6* is a reverse primer and may have any x as described above.
(Cn)ₓ-T3*, where n is 1 to 9 and x is 1 to about 30. In some embodiments, (Cn)ₓ T3* is (C3)₁-T3*, (C3)₂-T3*, (C3)₃-T3*, (C3)₄-T3*, (C3)₅-T3*, (C3)₆-T3*, (C3)₇- T3*, (C3)₈-T3*, (C3)₉-T3*, (C3)₁₀-T3*, (C3)₁₁-T3*, (C3)₁₂-T3*, (C3)₁₃-T3*, (C3)₁₄-T3*, (C3)₁₅-T3*, (C3)₁₆-T3*, (C3)₁₇-T3*, (C3)₁₈-T3*, (C3)₂₁-T3*, (C3)₂₄-T3*, (C3)₂₇-T3*, or (C3)₃₀ -T3*. In some embodiments, (Cn)ₓ-T3* is a forward primer and may have any x as described above. In other embodiments, (Cn)ₓ-T3* is a reverse primer and may have any x as described above.
(Cn)ₓ-GSO*, where n is 1 to 9, × is 1 to about 30, and GSO* is a gene specific oligonucleotide sequence, wherein the gene specific oligonucleotide comprises 50 or fewer nucleotides. In some embodiments, (Cn)ₓ GSO* is (C3)₁-GSO*, (C3)₂-GSO* ,(C3)₃-GSO *,(C3)₄-GSO*, (C3)₅-GSO*, (C3)₆-GSO*, (C3)₇-GSO*, (C3)₈-GSO*, (C3)₉-GSO *, (C3)₁₀-GSO*, (C3)₁₁-GSO*, (C3)₁₂-GSO*, (C3)₁₃-GSO*, (C3)₁₄-GSO *, (C3)₁₅-GSO*, (C3)₁₆-GSO*, (C3)₁₇-GSO*, (C3)₁₈-GSO*, (C3)₂₁-GSO*, (C3)₂₄-GSO*, (C3)₂₇-GSO*, or (C3)₃₀ -GSO*. In some embodiments, (Cn)ₓ-GSO* is a forward primer and may have any x as described above. In other embodiments, (Cn)ₓ-GSO* is a reverse primer and may have any x as described above.

The chemically enhanced primer includes a pre-determined number of nucleotides at its 3′terminus which are at least partially complementary to an equivalent number of nucleotides of the preceding sequence 5′ to the sequence of interest of the amplicon, which can then hybridize during the sequencing reaction to produce extension products of the chemically enhanced primer. In some embodiments, the 3′ pre-determined number of nucleotides of the chemically enhanced primer are at least partially complementary to a gene specific sequence at the 5′ terminus of the preceding sequence of the amplicon. In some embodiments, the 5′nucleotides of the preceding sequence of the amplicon, to which the 3′ pre-determined number of nucleotides of the chemically enhanced primer hybridizes, are not gene specific. When the 5′nucleotides of the preceding sequence of the amplicon are not a gene specific sequence, the 5′nucleotides of the preceding sequence may be any suitable tag, tail, universal sequence, bar code or ligation product.

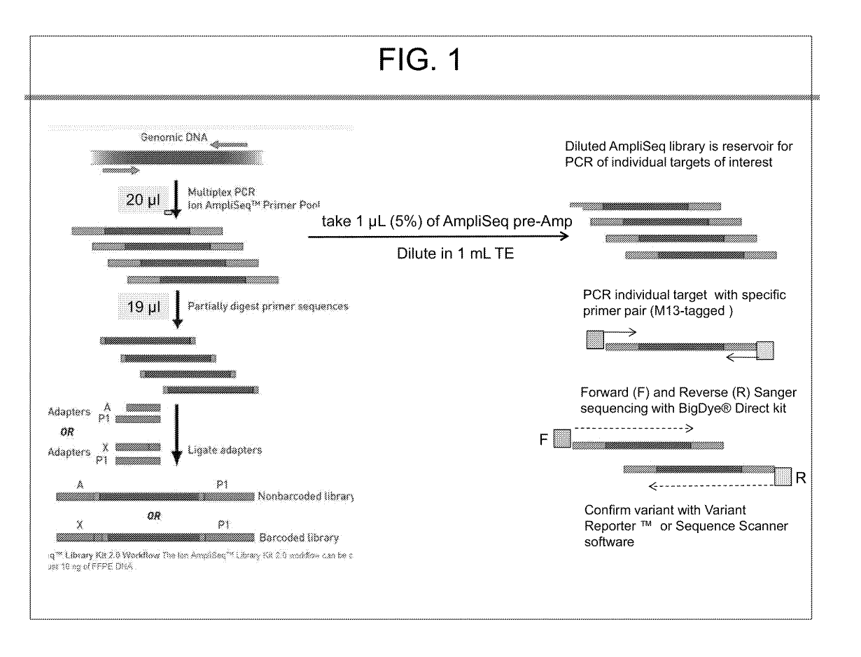

Using the chemically-enhanced sequencing primer, it has been found that high quality and highly accurate nucleic acid sequence results can be obtained in about 50% less time overall for a PCR to sequencing results workflow using POP7TM polymer on any of the 3130, 3730 or 3500 capillary electrophoresis platforms (Applied Biosystems, Foster City, Calif.). Specifically, improvement of 5′ sequence resolution is provided by use of the chemically enhanced primer which permits elucidation of base 1 from the chemically-enhanced sequencing primer. The throughput is increased as well as reducing hands-on-time by eliminating a separate PCR clean-up step prior to initiation of the sequencing reaction. (FIG. 1A and FIG. 1B). Overall, it can be seen that amplification, PCR clean-up and sequencing detection steps can each provide savings in run time, using differing aspects of the chemically-enhanced primers. The nuclease resistant aspect of the chemically-enhanced primers may provide reduced amplification and PCR clean-up time requirements, and the NCM aspect of the primers may allow efficacious separation in shorter run times, than standard sequencing primers can provide.

The chemically-enhanced sequencing primer and improved workflow improves polymorphism detection and more efficient use of allele specific sequencing primers for heterozygous ambiguity resolution. Various aspects of the use and synthesis of chemically-enhanced sequencing primers are further described in U.S. Application Ser. Nos. 61/026,085, filed Feb. 4, 2008; Ser. No. 12/365,140, filed Feb. 3., 2009; 61/407,899, filed Oct. 28, 2010; 61/408,553, filed Oct. 29, 2010; Ser. No. 13/284,839, filed Oct. 28, 2011; and Ser. No. 13/397,626, filed Feb. 15, 2012, and each disclosure of which is hereby incorporated by reference in its entirety.

Compositions. A composition for sequencing nucleic acid is described that includes: a PCR amplification reaction product that comprises: a DNA product amplified from at least one amplicon, wherein the amplicon comprises a sequence of interest and a preceding sequence 5′ to the sequence of interest incorporated from a first priming sequence; non-nuclease-resistant amplification primer(s); and a chemically enhanced primer wherein the chemically enhanced primer comprises an oligonucleotide sequence, a NCM and none or at least one nuclease-resistant linkage.

The chemically-enhanced primer may include a plurality of NCMs either at a terminal 5′ end or within a oligonucleotide sequence of the chemically-enhanced primer. The NCM may be a (Cn) spacer wherein n can be any integer from 1 to 9. In various embodiments, the NCM comprises a plurality of (Cn) spacers. In various embodiments, the chemically-enhanced primer may have a structure of Formula I:

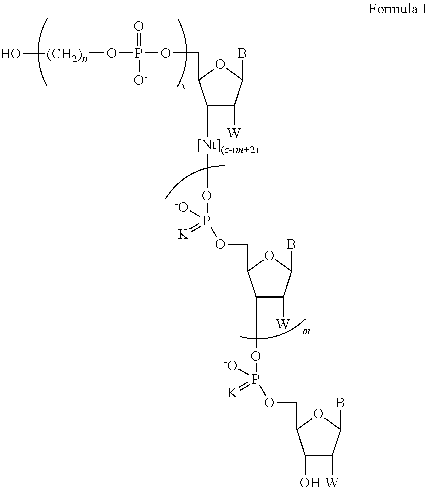

wherein B is a nucleobase; K is S or O; each n is independently an integer of 1 to 9; m is 0 or 1; × is an integer of 1 to about 30; z is an integer of 3 to about 100; W is OH, F, OMe, or H; and Nt is a moiety having a formula:

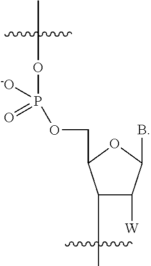

In some embodiments of the compositions of the invention, the chemically enhanced primer may be any chemically enhanced primer described in this disclosure.

In various embodiments, the oligonucleotide portion of the chemically-enhanced primer may include a universal primer. The universal primer may be selected from M13, US1, T7, SP6, and T3. The universal primer may be M13. In some embodiments, the chemically-enhanced primer may include one nuclease-resistant linkage.

In some embodiments of the compositions of the invention, the composition may further include a nuclease. In other embodiments, the composition may further include a polymerase, deoxynucleotide triphosphates, dideoxynucleotide triphosphates and a dye-label. In some embodiments, the dideoxynucleotide triphosphates may include dideoxynucleotide triphosphates labeled with the dye-label. The dye-labeled dideoxynucleotide triphosphates may include fluorescent dye-labeled dideoxynucleotide triphosphates. In some embodiments, the dye-label may be attached to the NCM or the oligonucleotide sequence.

In some embodiments of the compositions of the invention, the nuclease may be selected from exonuclease I, Exo III, Pfu and DNA pol I. In some embodiments, the polymerase may be Taq polymerase.

In some embodiments, the PCR amplification reaction product further includes an amplified DNA product where the DNA product is the amplification product of a plurality of amplicons.

In some embodiments, the composition for sequencing nucleic acids can further comprise more than one chemically-enhanced sequencing primer. In some embodiments, the polymerase can comprise Taq polymerase, for example AmpliTaq Gold polymerase. In some embodiments, the nuclease can comprise exonuclease I. In some embodiments, the chemically-enhanced sequencing primer can comprise at least one phosphorothioate linkage. In other embodiments, the chemically-enhanced sequencing primer can comprise a terminal 3′ end phosphorothioate linkage. In some embodiments the chemically-enhanced sequencing primer can comprise a dye, for example a fluorescent dye-labeled oliogonucleotide and/or at least one fluorescently dye-labeled NCM moiety within the NCM compound.

According to various embodiments, the composition for sequencing nucleic acid can comprise a polymerase, for example a DNA polymerase, in an amount of from about 0.01 Unit to about 20 Units, for example, from about 0.1 Unit to about 1.0 Unit, or about 0.8 Unit. The composition can comprise polymerase in an amount within a range having an upper limit of from about 10 Units to about 20 Units and a lower limit of from about 0.01 Unit to about 0.05 Unit. According to various embodiments, the composition can comprise a nuclease, for example exonuclease I, in an amount of from about 1 Unit to about 40 Units, for example, from about 2 Units to about 15 Units, or about 10 Units. The composition can comprise nuclease in an amount within a range having an upper limit of from about 10 Units to about 40 Units, and a lower limit of from about 1 Unit to about 2 Units.

According to various embodiments, the composition for sequencing nucleic acid can comprise a chemically-enhanced sequencing primer, in an amount of from about 0.1 μM to about 20 μM, for example about 1.0 μM. The composition can comprise a chemically-enhanced sequencing primer in an amount within a range having an upper limit of from about 10 μM to about 20 μM and a lower limit of from about 0.05 μM to about 0.1 μM. According to various embodiments, the composition can comprise dNTPs in an amount of from about 20 μM to about 5000 μM, for example, about 500 μM. The composition can comprise dNTPs in an amount within a range having an upper limit of from about 2000 μM to about 5000 μM and a lower limit of from about 20 μM to about 50 μM. According to various embodiments, the composition can comprise ddNTPs in an amount of from about 0.03 μM to about 10 μM, for example about 3 μM. The composition can comprise ddNTPs in an amount within a range having an upper limit of from about 5 μM to about 10 μM and a lower limit of from about 0.01 μM to about 0.05 μM. All molar amounts are based on final concentrations of the final volume.

According to various embodiments, the composition can comprise one or more non-nuclease-resistant amplification primers in an amount of from about 0.1 μM to about 20 μM each, for example about 0.01 μM or 1.0 μM. The composition can comprise one or more non-nuclease-resistant amplification primers in an amount within a range having an upper limit of from about 10 μM to about 20 μM each and a lower limit of from about 0.05 μM to about 0.1 μM each. All molar amounts are based on final concentrations of the final volume.

According to various embodiments, the composition for sequencing nucleic acid can further comprise a PCR amplification product. In some embodiments, the PCR amplification product can comprise an amplified DNA target sequence. In some embodiments, the PCR amplification product can comprise non-nuclease-resistant amplification primer(s). The non-nuclease-resistant amplification primer can comprise, for example, phosphodiester linkages that are sensitive to degradation by exonuclease. In some embodiments, the PCR amplification product can comprise a target specific amplicon that incorporates nucleic acid sequence capable of annealing to a universal primer.

Kits. The present teachings are also directed to kits that utilize the chemically-enhanced primer composition and methods described above. In some embodiments, a basic kit can comprise a container having one or more chemically-enhanced primers, as described in this disclosure. A kit can also optionally comprise instructions for use.

A kit is described which includes: a polymerase, a nuclease, at least one deoxynucleotide triphosphate, and dideoxynucleotide triphosphates. The dideoxynucleotide triphosphates may be dideoxynucleotide triphosphates labeled with a dye-label. The dye-labeled dideoxynucleotide triphosphates may be fluorescent dye-labeled dideoxynucleotide triphosphates.

In some embodiments, the nuclease may be selected from exonuclease I, Exo III, Pfu and DNA pol I. In various embodiments, the kit may include a chemically enhanced primer as described in this disclosure.

In other embodiments, the kit may further include a plurality of nuclease sensitive amplification primers. The plurality of nuclease- sensitive amplification primers may be configured to prime a sequence of interest of a specific disease state. The plurality of nuclease-sensitive amplification primers of the kit may be configured to prime a set of sequences connected to a specific disease state.

A kit can also comprise other optional kit components, such as, for example, one or more of a nuclease, a sufficient quantity of enzyme for sequencing or fragment analysis, buffer to facilitate the sequencing reaction or fragment analysis reaction , dNTPs, modified dNTPs, dNTP analogs and 7-Deaza-dGTP for strand extension during sequencing reaction or fragment analysis reaction, ddNTPs, a dye-label, loading solution for preparation of the sequenced or fragment analyzed material for electrophoresis, genomic DNA as a template control, a size marker to insure that materials migrate as anticipated in the separation medium, and a protocol and manual to educate the user and limit error in use. The amounts of the various reagents in the kits also can be varied depending upon a number of factors, such as the optimum sensitivity of the process. It is within the scope of these teachings to provide test kits for use in manual applications or test kits for use with automated detectors or analyzers. Kits may have more than one chemically enhanced primer, and the number of NCM moieties may be different in each of the chemically enhanced primers. Kits for plasmid sequencing may have any of the components listed above, but do not include a nuclease.

Examples. Examples of the compositions and methods of the present teachings are shown below. These examples are not limiting of the present teachings, and those of ordinary skill in the art will recognize that the components used in the reactions may be readily substituted with equivalent reagents known in the art.

### EXAMPLE 1

C6 spacer+Oligo seq. synthesis, no phospohorothioate group: An 18 base oligonucleotide labeled with one or more C6 spacers at the 5′ position was made on an ABI model 394 DNA synthesizer using standard phosphoramidite chemistry. The C6 spacer phosphoramidite was obtained from Chem Genes Corp. (P/N CLP-1120, Wilmington, Mass.). The labeled 18mer was made with the trityl group intact from a one micromole column. On completion of the synthesis the oligonucleotide was cleaved off the support with NH₄OH and purified by HPLC using an ABI RP-300 (C-8) column (4.6×220 mm) using a flow rate of 1.5 ml/min. and a solvent gradient of 0.1M triethylarnmoniurn acetate-water pH 7.0 and acetonitrile, the trityl group was removed and the product was isolated by ethanol precipitation.

C3 spacer +Oligo seq. synthesis, no phosphorothioate group: An 18 base oligonucleotide labeled with one or more C3 spacers (P/N 104913-90, Glen Research), at the 5′ position was made on an ABI model 394 DNA synthesizer using standard phosphoramidite chemistry. The labeled 18mer was made with the trityl group intact from a one micromole column. On completion of the synthesis the oligonucleotide was cleaved off the support with NH₄OH and purified by HPLC using an ABI RP-300 (C-8) column (4.6×220 mm) using a flow rate of LS ml/rein. and a solvent gradient of 0.1M triethylammonium acetate-water pH 7.0 and acetonitrile, the trityl group was removed and the product was isolated by ethanol precipitation.

Protocol for oligo labeled with one or more C-3 spacer containing a 3′ phosphorothioate linkage: An 18 base oligonucleotide labeled with one or more C-3 spacers at the 5′ position was made on an ABI model 394 DNA synthesizer using standard phosphoramidite chemistry. The 3′ phosphorothioate linkage was made using standard methods with sulfurizing reagent (TEM P/N 401267 (Applied Biosystems, Foster City, Calif.). The C3 spacer phosphorainidite was obtained from Glen Research (P/N 10-1913-90). The labeled 18mer was made with the trityl group intact from a one micromole synthesis column. On completion of the synthesis the oligonucleotide was cleaved off the support with NH₄OH and purified by HPLC using an ABI RP-300 (C-18) column (4.6×220 mm) using a flow rate of 1.5 inl/rnin. and a solvent gradient of 0.1M triethyiammonium acetate-water pH TO and acetonitrile, the trityl group was removed and the product was isolated by ethanol precipitation. Note: To synthesize more than one phosphorothioate linkage or to place this linkage anywhere in the 18-mer oligonucleotide chain, oxidize using the sulfurizing reagent at these position(s).

### EXAMPLE 2

A minor fraction (e.g. 5%) of original pre-amplication material from the Ion AmpliSeq comprehensive cancer panel v2 (CHP v2) and the Ion Oncomine cancer panel (OCP) is used for follow up (confirmatory) analysis using traditional fluorescent dye terminator sequencing a.k.a. Sanger sequencing and detection by automated capillary electrophoresis (CE) such as the Applied Biosystems 3500 XL Genetic Analyzer.

Molecular analysis of genetic mutations (variants) in tumor samples is becoming increasingly used for tumor characterization and the diagnostic and therapeutic management of cancer patients. Often, only very limited amount of tumor specimen is initially (i.e. pre-surgery) available for example by fine needle biopsy of suspected tumor tissue or aberrant cell clusters present in a formalin fixed paraffin embedded (FFPE) preparations.

The Ion AmpliSeq™ cancer panels are designed to amplify a multitude of oncologically relevant target genes from low amount of input DNA (10 ng) for subsequent sequencing on a chip-based platform (e.g. the Ion Torrent PGM™ instrument). The Ion Ampliseg™ CHPv2 panel covers 207 loci and the OCP panel over 2000 targets. These panels and disclosure related to loci selection, amplicon size, and primer development are further described in U.S. Application Ser. Nos. 61/479,952, filed on Apr. 28, 2011; 61/531,583, filed on Sep. 6, 2011; 61/531,574, filed on Sep. 6, 2011; 61/538,079, filed on Sep. 22, 2011; 61/564,763, filed on Nov. 29, 2011; 61/578,192, filed on Dec. 20, 2011; 61/594,160, filed on Feb. 2, 2012; 61/598,881, filed on Feb. 14, 2012; 61/598,892, filed on Feb. 14, 2012; 61/625,596, filed on Apr. 17, 2012; 61/639,017, filed on Apr. 26, 2012; Ser. No. 13/458,739, filed on Apr. 27, 2012; Ser. No. 13/663,334, filed on Oct. 29, 2012; Ser. No. 13/679,706, filed on Nov. 16, 2012; and an application entitled “Detection, Identification, Validation, and Enrichment of Target Nucleic Acids”, Attorney Docket No. LT00974 PRO, filed on even date; and each disclosure is hereby incorporated by reference in its entirety.

Occasionally, sequencing results obtained from the next generation sequencing (NGS) platform need to be confirmed with an orthogonal methodology such as traditional Sanger sequencing. Such a reflex test is indicated when a minor variant i.e. a coding or functionally relevant nucleotide variant occurs not in a 50% frequency as expected if inherited in a Mendelian fashion, but rather at a lower frequency (i.e. between 5-25%) which are typical for somatic mutations i.e. a mutational event that happens spontaneously or is causative for or driving carcinogenesis.

For a pair of forward and reverse Sanger sequencing reactions of a single target 5 ng of genomic DNA is typically needed which may quickly exhaust the amount available from the clinical specimen.

Therefore, it is desirable to exploit the enriched, pre-amplified, pool of target sequences from the Ampliseq reaction for re-sequencing by Sanger.

Typically an Ampliseg™ pre-amplification reaction is set up to be performed in a reaction volume of 20 uL. Removing an aliquot of 1 ul (i.e. 5%) of this material prior to the primer trimming step (see FIG. 1) as a potential reserve for reflex testing by Sanger sequencing is not detrimental for the subsequent steps in the NGS sequencing library preparation process. Alternatively, a user could set up an initial AmpliSeq™ reaction with a volume of 21-22 ul and then remove 1 uL after pre-amplification.

The typical input amount of human DNA that goes into an AmpliSeq™ reaction is 10 ng which is the equivalent of 3000 genome copies or 1500 cells. The preamplication conditions are:

TABLE 2

| DNA type | CHP v2 | OCP 1 & 2 |
|---|---|---|
| Purified gDNA | 17 cycles | 15 cycles |
| FFPE DNA | 20 cycles | 18 cycles |

Table 1: Number of pre-amplification cycles for Ampliseq panel and material used. Cycling conditions are: 99 C 2 min (1×) then [99 C 15 sec , 60 C 4 min] for # of cycles shown in Table 1.
Pre-amplification material is derived from 3 DNA sources:
80:20 is a mixture (80%:20%) of 2 Coriell DNAs
FFPE 1 (DNA extracted from FFPE specimen “1”)
FFPE 5 (DNA extracted from FFPE specimen “5”)

A 1 ul aliquot of the preamplification material (PA) is diluted 1:1000 in 1 ml TE buffer.

Assuming 100% efficiency in the pre-amplification PCR reaction the number of targets can be calculated that go into the subsequent PCR reaction for Sanger Sequencing (BDD PCR)

TABLE 3

| | | | | # of targets in |
|---|---|---|---|---|
| | # of targets in | | | BDD PCR reaction |
| | a AmpliSeq | | | when AS PA is |
| | reaction after | | | diluted 1000-fold |
| | pre-amp assuming | | # of | and 0.5 ul is used |
| | 100% efficiency | | targets/ul | as template in PCR |
| 80:20 CHP2 | 393216000 | =3000*(1 + 1)\hat{ }17 | 19660800 | 9830 |
| 80:20 OCP1 | 98304000 | =3000*(1 + 1)\hat{ }15 | 9830400 | 4915 |
| 80:20 OCP2 | 98304000 | =3000*(1 + 1)\hat{ }15 | 9830400 | 4915 |
| FFPE 1 CHP2 | 3145728000 | =3000*(1 + 1)\hat{ }20 | 157286400 | 78643 |
| FFPE 1 OCP1 | 786432000 | =3000*(1 + 1)\hat{ }18 | 78643200 | 39322 |
| FFPE 1 OCP2 | 786432000 | =3000*(1 + 1)\hat{ }18 | 78643200 | 39322 |
| FFPE 5 CHP2 | 3145728000 | =3000*(1 + 1)\hat{ }20 | 157286400 | 78643 |
| FFPE 5 OCP1 | 786432000 | =3000*(1 + 1)\hat{ }18 | 78643200 | 39322 |
| FFPE 5 OCP2 | 786432000 | =3000*(1 + 1)\hat{ }18 | 78643200 | 39322 |

Due to the pre-amplification reaction there is sufficient target materials available for reflex testing. The previously target-limited sample is not limited anymore. Besides Sanger sequencing also other detection methods can be potentially employed, including but not limited to digital PCR, cast-PCR or other allele-specific PCR methods, single base extension chemistry and detection (by CE or Mass spectroscopy).

Further, panels of informative or actionable targets (e.g. TP53, KRAS, BRAF, EGFR, etc.) can be designed that can be analyzed by Sanger Sequencing as a first step prior to next gen sequencing or used exclusively by Sanger sequencing (or another method). PCR primers used:

The amplicon and primer sequences of the CHPv2 panel were evaluated for general suitability for singular PCR. From this list, a total of 48 targets (including 18 “difficult” targets, having GC rich regions, short amplicon regions, hompolymer A or homopolymer T stretches) and their corresponding PCR primer pairs and added M13 Forward sequence (tgtaaaacgacggccagt) to the 5′ end of the AmpliSeq forward primer and M13 reverse sequence (caggaaacagctatgacc) to the 5′ end of the AmpliSeq reverse primer. These are nuclease sensitive amplification primers for use in the methods described in this disclosure. Primer data are listed in Table 8, following this section.

Primers are delivered resuspended in water at a concentration of 100 μM. A Primer Pair (PP) master plate is generated by combining 10 ul of each corresponding (Forward and reverse) primer pair and then diluted with 80 uL low TE to 100 uL so that the primers in the pair were at a concentration of 10 μM.

The orientation of the primer pairs on the master plate is as follows:

Primer Layout on Plate

From the PP Master plate test plates are generated by diluting the 10 uM stock primer pairs further to 1 uM in low TE and plated 10 uL of the diluent to two sets (CHPv2 A and B) of 96 well plates in the following composition:

Each plate contains thus 4 sections of 24 identical primer pairs allowing the processing of 4 samples which were typically:
i) CEPH-1347 genomic DNA (control DNA from BigDye Direct sequencing kit diluted to 1 ng/ul in TE); this sample was typically located in columns 1-3
ii) NA 80:20 material (pre-amplified in CHPv2 or OCP (1&2) was always located in columns 4-6
iii) FFPE 1 material (pre-amplified in CHPv2 or OCP (1&2) was located in in columns 7-9
iv) FFPE 5 material (pre-amplified in CHPv2 or OCP (1&2) was located in columns 10-12

Primers were allowed to dry in situ on the plate and then used within 2 weeks for PCR Sequencing experiments using the BigDye® Direct Sequencing Kit, which includes reagents for PCR amplification and subsequent cycle sequencing chemistries.

The BigDye® Direct Sequencing Kit uses M13-tagged PCR primers. This is advantageous because in the subsequent sequencing reaction chemically enhanced primers, as described in the sections above, and having a sequence of M13 forward or reverse, are used as sequencing primers. This allows sequence reading almost immediately at the 5′end of the PCR amplicon. This maximizes the sequence information which is important since the AmpliSeq primers are designed to be fairly short (125-175 nt) owing to the short nature of heavily fragmented FFPE DNA.

After PCR, BigDye® Direct Sequencing reagent is added to the amplification reaction product in situ in the amplification mixture along with the chemically enhanced sequencing primer(s). This reagent contains not only the typical reagents needed for cycle sequencing (a polymerase, dNTPs, and dye-labelled ddNTPs). , but also contains nuclease to remove the need for additional purification manipulations by removing excess amplification primers in situ.

TABLE 6
PCR and sequencing conditions

| | |
|---|---|
| for 1 | |
| sample | for 24 samples |

| | | | | |
|---|---|---|---|---|
| 6.5 | uL | 160 | uL | BDD PCR reagent |
| 6.5 | uL | 160 | uL | dH20 |

| | | | |
|---|---|---|---|
| 0.5-1 | uL | 12 (up to 20 uL) | diluted Pre-Amp material |

add 13 uL to each well in 3 columns A-H (24 wells) in test plate (containing arrayed dried down primer pairs)
PCR in Veriti Fast thermal cycler:
I: 94 C 10 min (1×)
II: 95 C 3 sec,60C 15 sec,68C 45 sec (8 cycles)
III: 95 C 3 sec,70C 50 sec (28 cycles)
Prepare 2 Sequencing Mixes: F (Forward) and R (Reverse)

TABLE 7

| for 1 | | |
|---|---|---|
| sample | for 96 samples | |
| 1.3 | 130 | BDD Sequencing Reagent |
| 0.65 | 65 | BDD M13 Primer (Forward or |
| | | Reverse) |

add 2 uL Forward sequencing mix to each well in a fresh Fast PCR plate
add 6.3 ul PCR material from PCR plate: Forward SEQ
to remainder of PCR plate (˜6.3 ul PCR material left)
add 2 ul Reverse Sequencing Mix : Reverse SEQ
PCR in Veriti Fast thermal cycler:
37 C 20 min (1×)
80 C 2 min (1×)
95 C 1 min (1×)
95 C 3 sec; 50 C 5 sec; 60 C 45 sec (27 cycles)

Sequencing reactions were purified by addition of 55 ul BigDye® Xterminator beads solution mix followed by 30 min vigorous vortexing, to remove smaller molecule contaminants. After spinning the beads to the bottom of the well the plates were put into a Applied Biosystems 3500 XL Genetic Analyzer for capillary electrophoresis and sequence base calling. The resulting .ab1 sequencing files were quality assessed with Applied Biosystems Sequence Scanner software and then further analyzed using Applied Biosystems Variant Reporter software for detection of variants.

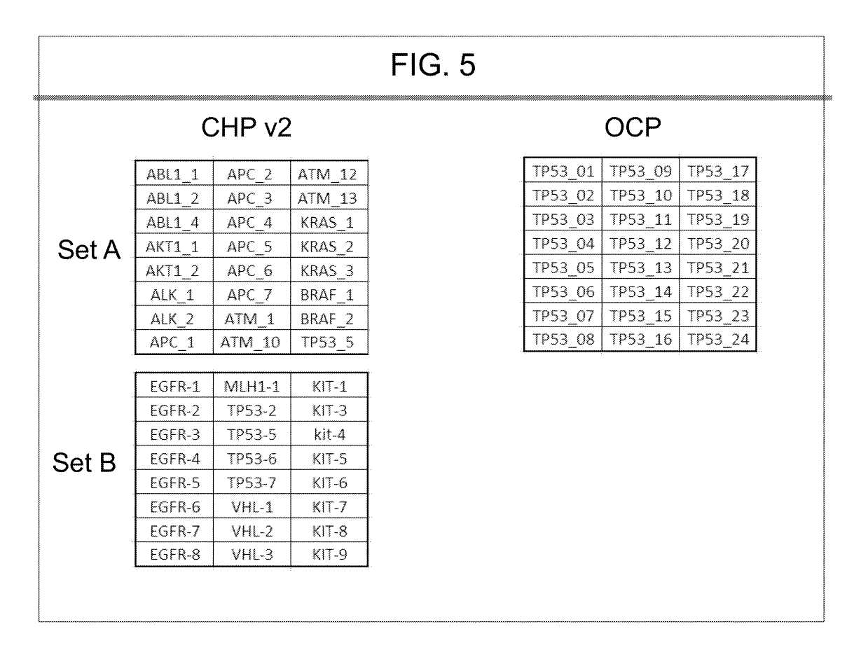

FIG. 5 shows the specific targets of the verification assays performed by Sanger re-sequencing and in particular BigDye® Direct sequencing techniques. CHP v.2 indicates that those loci are part of the Ion AmpliSeq™ Cancer Hotspot Panel v.2 and OCP indicates that the indicated loci are part of the Ion Oncomine™ cancer panel.

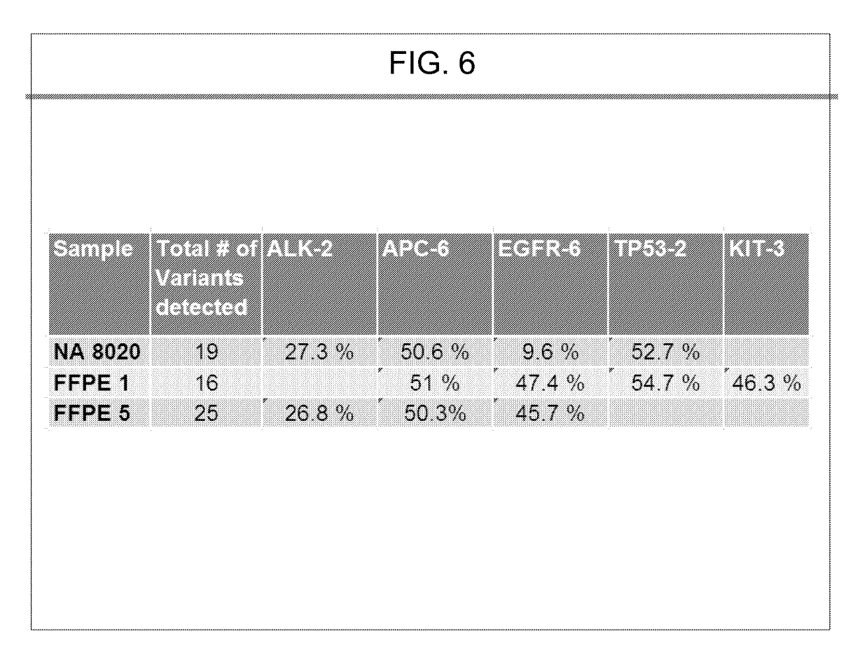

FIG. 6 shows the variants found arising from three samples, using Ion AmpliSeq methodology on the Ion PGM™ (318 chip). The second column indicates the number of variants found in the specific sample. The remaining columns to the right indicate, for a specific loci, percentage observed for a variant sequence.

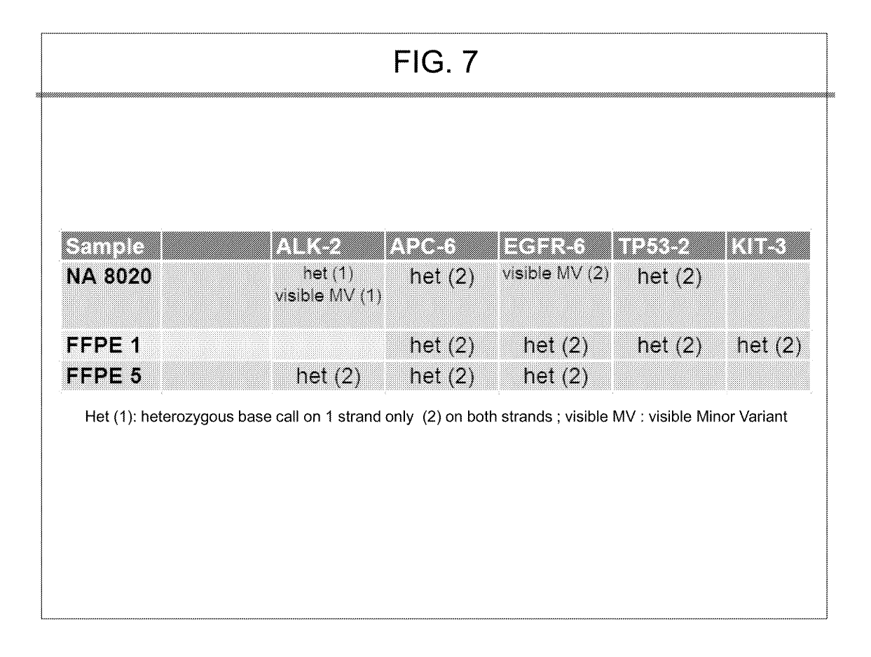

FIG. 7 shows verification of the variant sequences found from the same three samples as that of FIG. 6, upon resequencing using the methods of the invention, via Sanger sequencing. The same loci are interrogated and variants are confirmed.

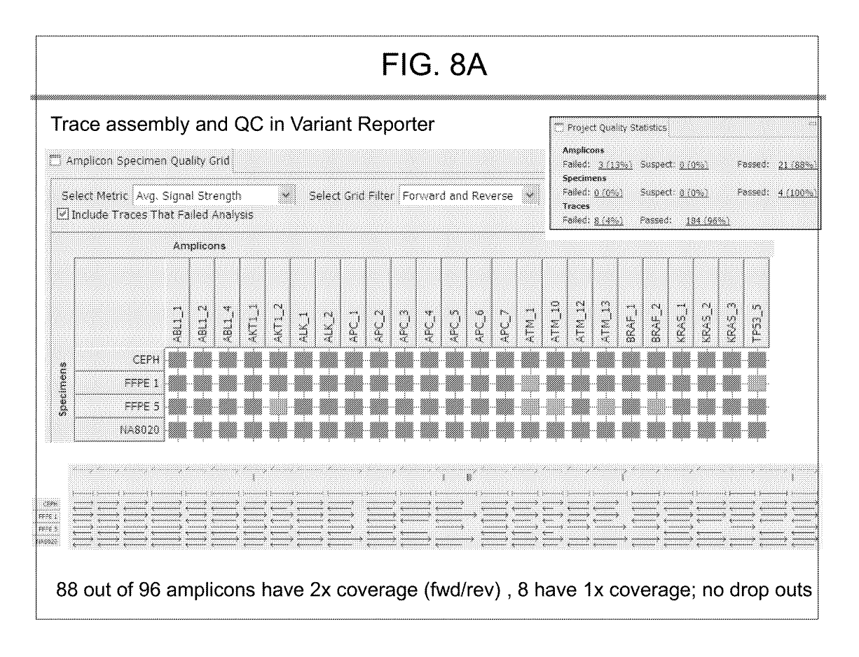

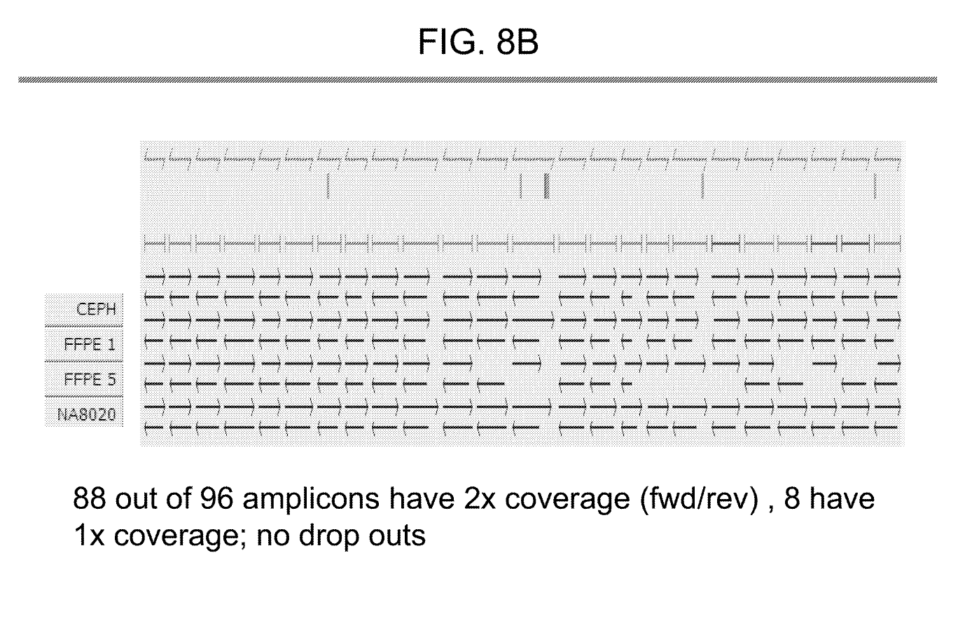

FIGS. 8A-8B are schematic representations of the Quality Grid (as seen in Applied Biosystems Variant Reporter™ software) for Target Sanger CE Test Set A for CHP v2 PA of FIG. 5. The lower panel of FIG. 8A is reproduced in larger scale in FIG. 8B, and demonstrates for each of four very limited originating samples taken through the workflow from AmpliSeq to Sanger Sequencing, that 88 out of 96 resulting amplicons have 2× coverage (fwd/rev), and 8 have 1× coverage. There are no drop outs. Right facing arrow indicates successful forward extension product production and left facing arrow indicates successful reverse extension product production.

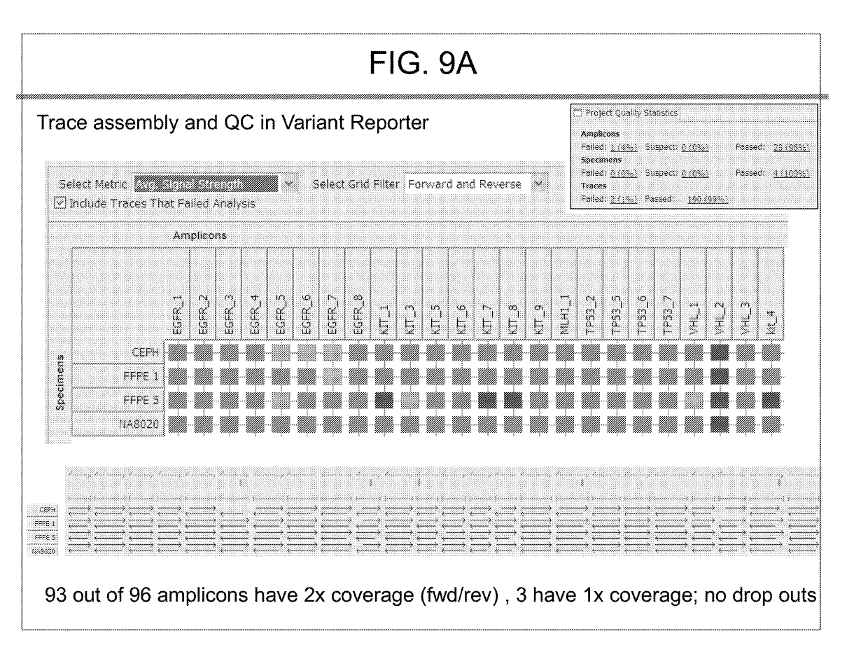

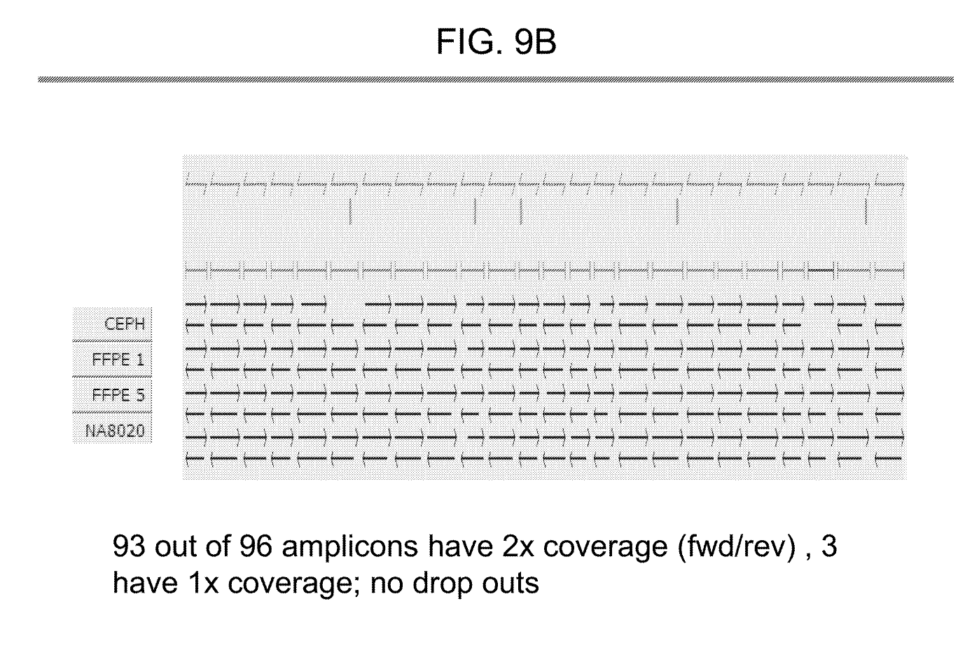

FIGS. 9A-9B are schematic representations of the Quality Grid (as seen in Applied Biosystems Variant Reporter™ software) for Target Sanger CE Test Set B for CHP v2 PA of FIG. 5. The lower panel of FIG. 9A is reproduced in larger scale in FIG. 9B, and demonstrates for each of four very limited originating samples taken through the workflow from AmpliSeq to Sanger Sequencing, that 93 out of 96 amplicons have 2× coverage (fwd/rev), and 3 have 1× coverage. There are no drop outs. Right facing arrow indicates successful forward extension product production and left facing arrow indicates successful reverse extension product production.

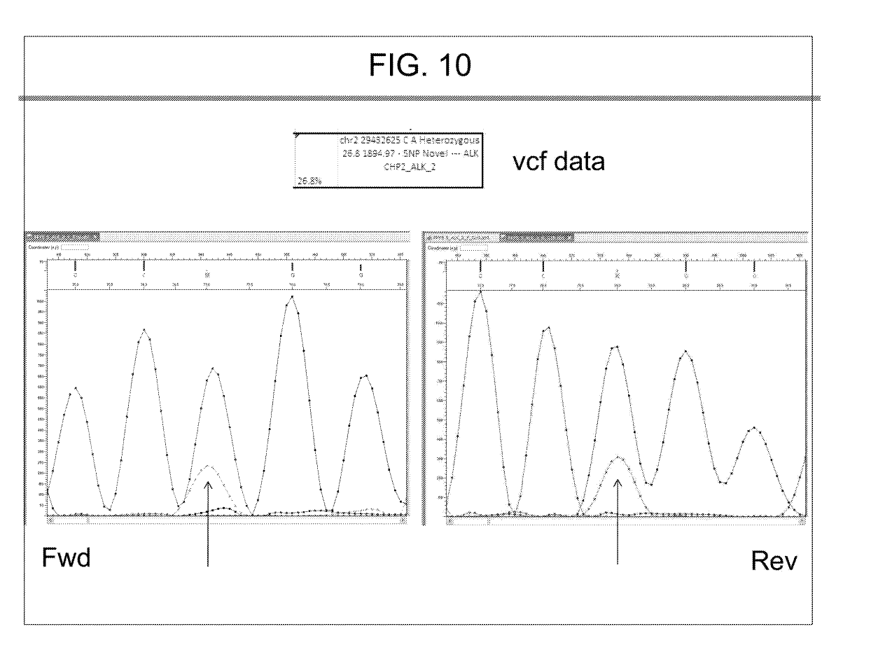

FIG. 10 shows the electropherogram demonstrating the sequencing results detecting a minor variant in ALK-2 for sample FFPE-5. The arrows in the left panel (forward sequence) and right panel (reverse sequence) clearly show a significant amount of minor variant under the major variant signal peak, which can be called by KB™ basecaller as a mixed base. This visual ratio can be compared to the ratio provided for the AmpliSeq derived results obtained by use of Ion Torrent Suite™ software to analyze the ratio of minor to major, which assigns a 26.8% ratio for the minor variant.

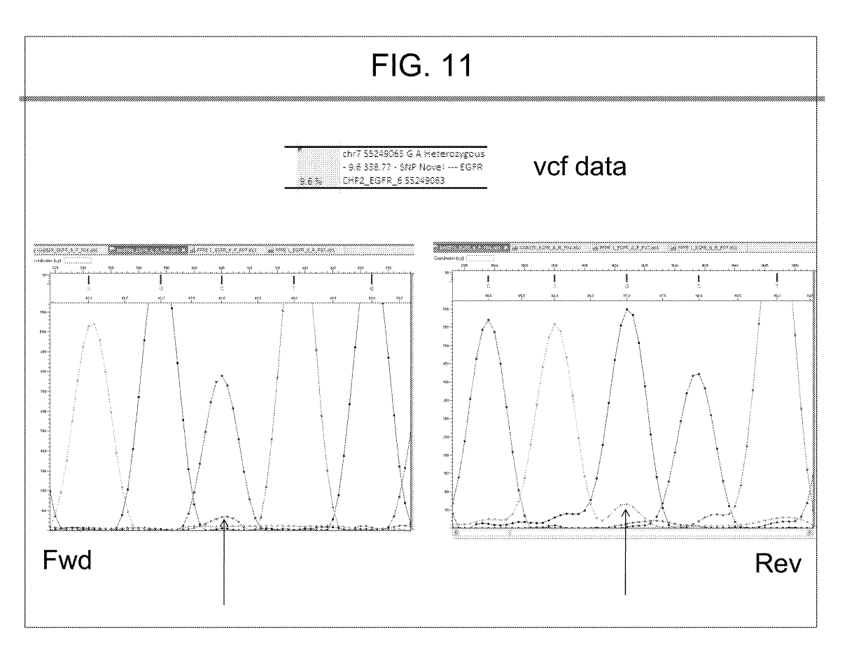

FIG. 11 shows the electropherogram demonstrating the sequencing results detecting a minor variant in EGFR-6 for sample NA 8020. The arrows in the left panel (forward sequence) and right panel (reverse sequence) clearly show a detectable amount of minor variant under the major variant signal peak, while it could not be called by KB™ basecaller as a mixed base. This visual ratio can be compared to the ratio provided for the AmpliSeq derived results obtained by use of Ion Torrent Suite™ software to analyze the ratio of minor to major, which assigns a 9.6% ratio for the minor variant.

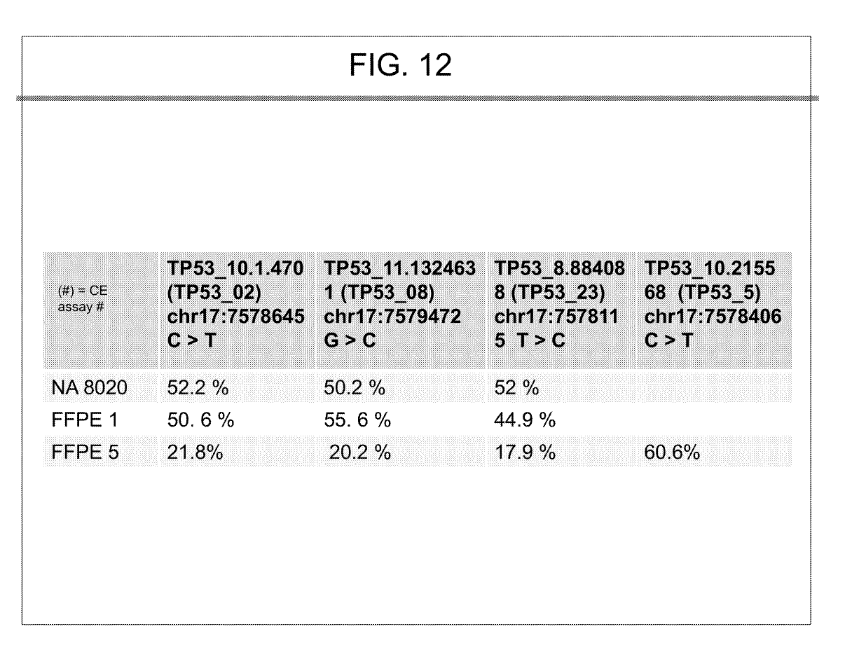

FIG. 12 is a schematic representation of the frequency of TP53 mutations found from sequencing of three samples using OCP AmpliSeq™ on the Ion PGM™ (318 chip).

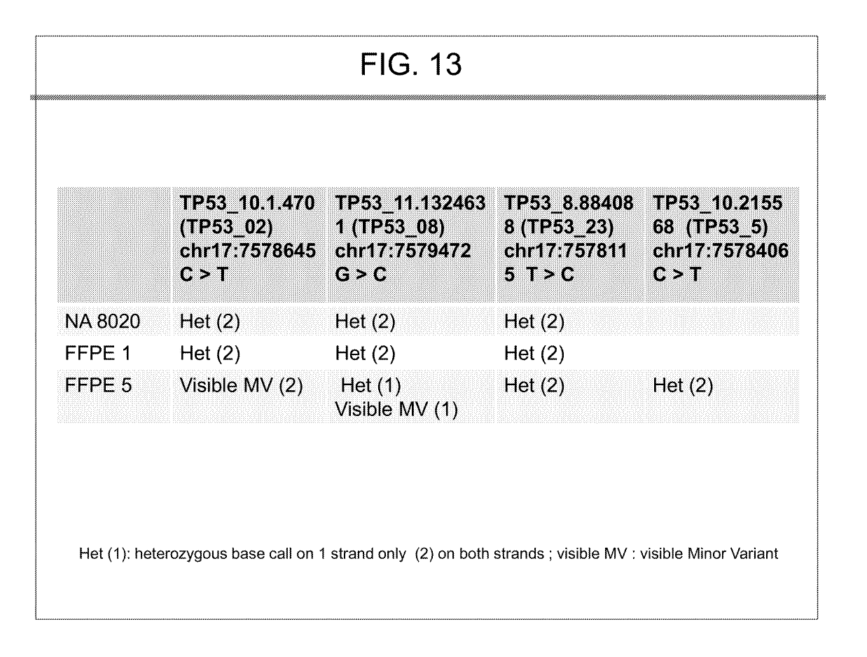

FIG. 13 is a schematic representation of the resequenced samples of FIG. 12, using the methods of the invention to verify the TP53 mutations shown in FIG. 12.

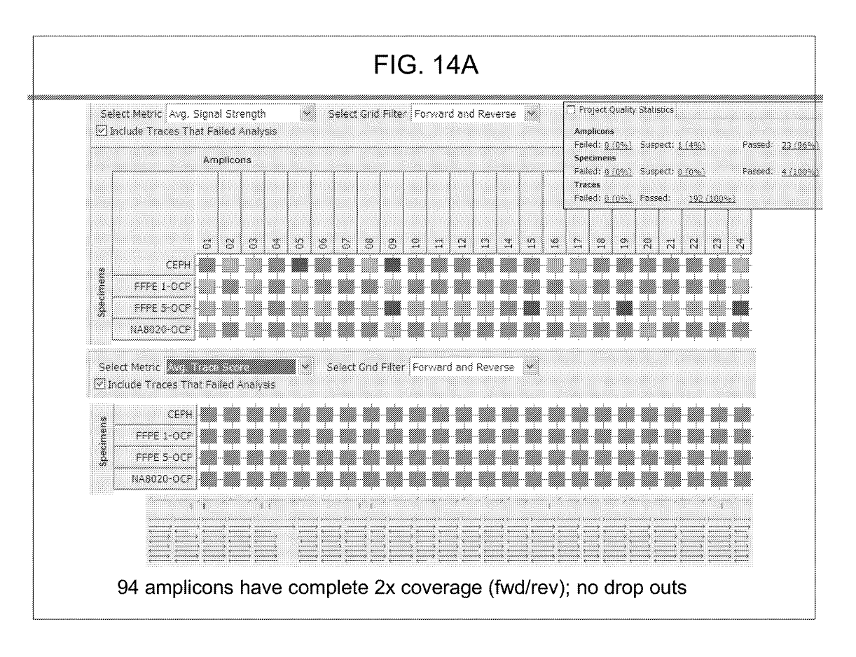

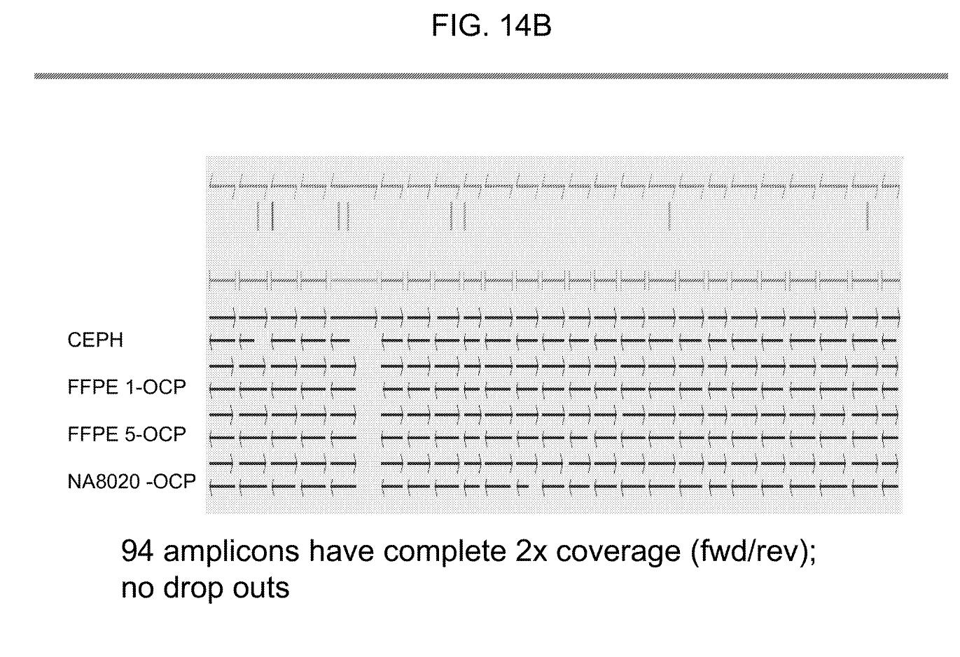

FIGS. 14A-14B show the Quality Grid (as seen in Applied Biosystems Variant Reporter™ software) for 24 TP53 Individual Amplicons from OCP Ampliseq™, for four samples. The lower panel of FIG. 14A is reproduced in larger scale in FIG. 14B, and demonstrates for each of four very limited originating samples taken through the workflow from AmpliSeq™ to Sanger Sequencing, that 94 of 96 amplicons have complete 2× coverage (fwd/rev). There are no drop outs. Right facing arrow indicates successful forward extension product production and left facing arrow indicates successful reverse extension product production.

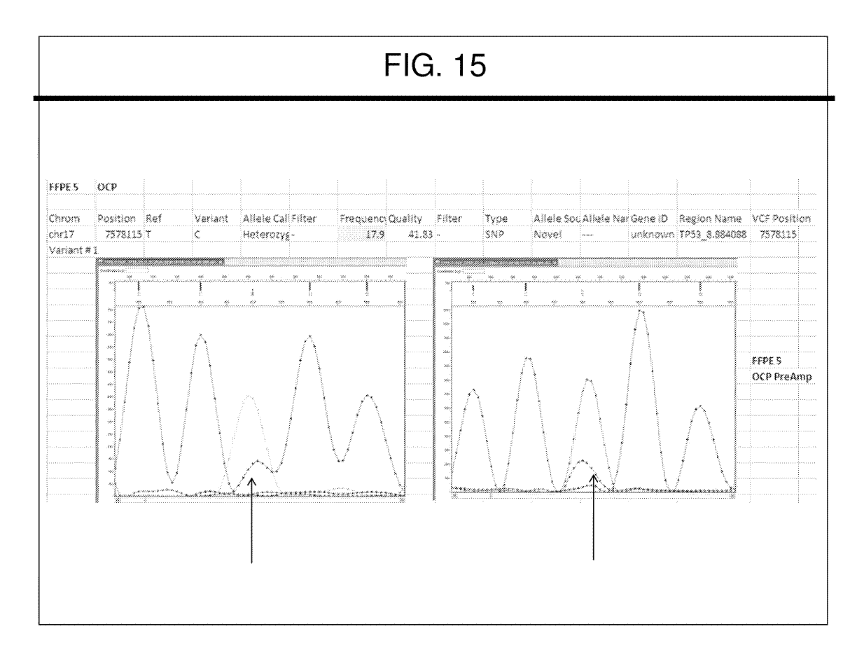

FIG. 15 shows the electropherogram of the sequencing results detecting a minor variant in TP53 for sample FFPE 5. The arrows in the left panel (forward sequence) and right panel (reverse sequence) clearly show a detectable amount of minor variant under the major variant signal peak. This visual ratio can be compared to the ratio provided for the AmpliSeq derived results obtained by use of Ion Torrent Suite™ software to analyze the ratio of minor (C) to major (T), which assigns a 17.9% ratio for the minor variant.

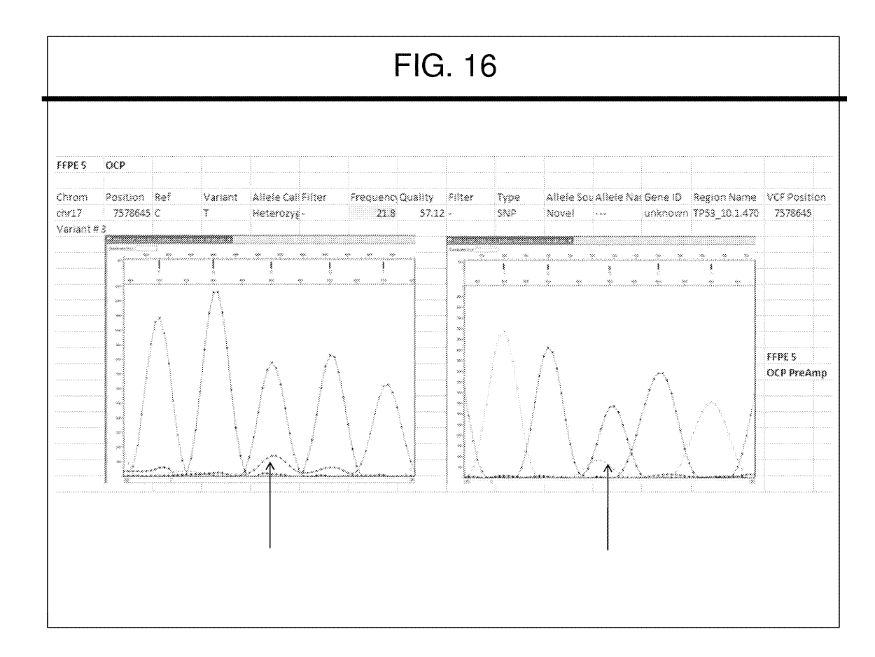

FIG. 16 shows the electropherogram of the sequencing results detecting a minor variant in TP53 at a different position from that shown in FIG. 15, for sample FFPE 5. The arrows in the left panel (forward sequence) and right panel (reverse sequence) clearly show a detectable amount of minor variant under the major variant signal peak. This visual ratio can be compared to the ratio provided for the AmpliSeq derived results obtained by use of Ion Torrent Suite™ software to analyze the ratio of minor (T) to major (C) assigns a 21.8% ratio for the minor variant.

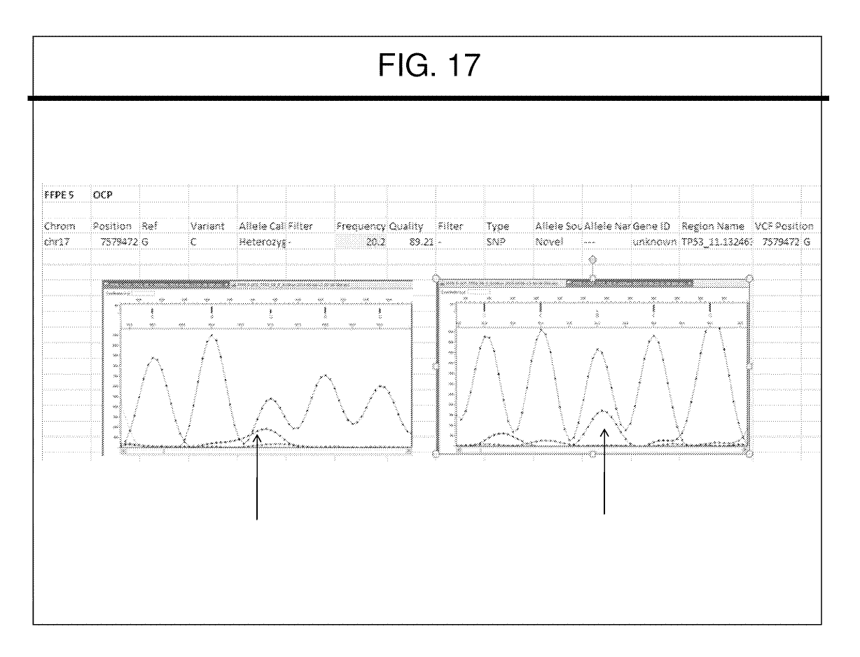

FIG. 17 shows the electropherogram of the sequencing results detecting a minor variant in TP53 at yet a third position from that shown in FIG. 15, for sample FFPE 5. The arrows in the left panel (forward sequence) and right panel (reverse sequence) clearly show a detectable amount of minor variant under the major variant signal peak. This visual ratio can be compared to the ratio provided for the AmpliSeq derived results obtained by use of Ion Torrent Suite™ software to analyze the ratio of minor (C) to major (G) assigns a 20.2% ratio for the minor variant.

### EXAMPLE 3

### Capillary Electrophoresis Sample Preparation and Detection

The amplified samples are analyzed by methods that resolve nucleobase sequences as would be known to one of skill in the art. For example, capillary electrophoresis can be used following the instrument manufactures directions. BigDye XTerminator Purification Kit (Applied Biosystems, P/N 4376486) can be used in cycle sequencing clean up to prevent the co-injection of un-incorporated dye-labeled terminators, dNTPs and salts with dye-labeled extension products into a capillary electrophoresis DNA analyzer. Briefly, 13 μL sequencing reaction mixture was combined with 45 μL SAM Solution and 10 μL XTerminator Solution. After vortexing the sample plate at 1800 rpm for 20 minutes, spin the plate at 1000×g for 2 minutes. To each well was added 30 μL of 70% ethanol and the plate was centrifuged at 1650×g for 15 minutes. The solution was removed by inverting the plate onto a paper towel and centrifuging at 180×g for 1 minute. The precipitated sequencing reaction was then dissolved in 10 μL of 50 μM EDTA and loaded onto an AB 3500xL Genetic Analyzer equipped with a 50 cm capillary array (Applied Biosystems, Foster City, Calif.).

### EXAMPLE 4

### Capillary Electrophoresis Methods and Analysis

Capillary electrophoresis (CE) was performed on the current Applied Biosystems instruments, for example the Applied Biosystems 3500xl Genetic Analyzer, using the dye set Z as described the instrument's User Guide. There are ShortReadSeq_BDX_POP7, RapidSeq_BDX_POP7, FastSeq_BDX_POP7, StdSeq_BDX_POP7 run modules. For example, BDxFastSeq50_POP7xl_1 parameters were: oven temperature: 60C, sample injection for 5 sec at 1.6 kV and electrophoresis at 13.4 kV for 2520 sec in Performance Optimized Polymer (POP-7™ polymer) with a run temperature of 60° C. Variations in instrument parameters, e.g. injection conditions, were different on other CE instruments such as the 3500 or 3730x1 Genetic Analyzers. The data were collected using versions the Applied Biosystems Data Collection Software specific to the different instruments, such as 3500 Data Collection Software v1.0. The sequence traces were analyzed by Applied Biosystems KB™ Basecaller Software v1.4.1 with KB_3500_POP7_BDTv3direct.bcc and KB_3500_POP7_BDTv3direct.mob to determine the correct base calls.

Those skilled in the art understand that the detection techniques employed are generally not limiting. Rather, a wide variety of detection means are within the scope of the disclosed methods and kits, provided that they allow the presence or absence of an amplicon to be determined. While the principles of this invention have been described in connection with specific embodiments, it should be understood clearly that these descriptions are made only by way of example and are not intended to limit the scope of the invention. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

TABLE 8

| Ampliseq | chromosome: | | | amplicon |
|---|---|---|---|---|
| ID | location | Forward Primer | Reverse Primer | length |
| CHP2_ABL1 | >chr9: | TCTATGGTGTGTCCCCCAACT | CGTCAGGCTGTATTTCTTCC | 128 |
| _1 | 133738274 + | (SEQ ID NO: 6) | ACA (SEQ ID NO: 7) | |
| | 133738401 | | | |
| CHP2_ABL1 | >chr9: | CGCTGAAGCTCCATTTTGCAT | CAGCTTCTTTCAAGAACTCT | 134 |
| _2 | 133747422 + | (SEQ ID NO: 8) | TCCAC (SEQ ID NO: 9) | |
| | 133747555 | | | |
| CHP2_ABL1 | >chr9: | GGAGAACCACTTGGTGAAGGT | CGGACTTGATGGAGAACTT | 146 |
| _4 | 133750285 + | AG (SEQ ID NO: 10) | GTTGTA | |
| | 133750430 | | (SEQ ID NO: 11) | |
| CHP2_AKT | >chr14: | GCGCCACAGAGAAGTTGTTGA | GGGTCTGACGGGTAGAGT | 179 |
| 1_1 | 105246425 + | (SEQ ID NO: 12) | GT (SEQ ID NO: 13) | |
| | 105246603 | | | |
| CHP2_AKT | >chr14: | CTTGGCCACGATGACTTCCTT | CCATGAACGAGTTTGAGTA | 131 |
| 1_2 | 105241413 + | (SEQ ID NO: 14) | CCTGA | |
| | 105241543 | | (SEQ ID NO: 15) | |
| CHP2_ALK_ | >chr2: | TCTCTCGGAGGAAGGACTTGA | GCCCAGACTCAGCTCAGTT | 166 |
| 1 | 29443586 + | G (SEQ ID NO: 16) | AAT (SEQ ID NO: 17) | |
| | 29443751 | | | |
| CHP2_ALK_ | >chr2: | ACAGGGTACCAGGAGATGATG | GGAAGAGTGGCCAAGATT | 154 |
| 2 | 29432548 + | TAAG (SEQ ID NO: 18) | GGA | |
| | 29432701 | | (SEQ ID NO: 19) | |
| CHP2_APC | >chr5: | GAGAGAACGCGGAATTGGTCT | GTATGAATGGCTGACACTT | 138 |
| _1 | 112173850 + | A (SEQ ID NO: 20) | CTTCCA | |
| | 112173987 | | (SEQ ID NO: 21) | |
| CHP2_APC | >chr5: | AGCACTGATGATAAACACCTCA | ATCTTCTTGACACAAAGAC | 159 |
| _2 | 112174532 + | AGTT (SEQ ID NO: 22) | TGGCT | |
| | 112174690 | | (SEQ ID NO: 23) | |
| CHP2_APC | >chr5: | TTCATTATCATCTTTGTCATCAG | TTTGGTTCTAGGGTGCTGT | 175 |
| _3 | 112175116 + | CTGAA (SEQ ID NO: 24) | GAC (SEQ ID NO: 25) | |
| | 112175290 | | | |
| CHP2_APC | >chr5: | GCAGACTGCAGGGTTCTAGTT | GTGAACTGACAGAAGTACA | 174 |
| _4 | 112175295 + | (SEQ ID NO: 26) | TCTGCT | |
| | 112175468 | | (SEQ ID NO: 27) | |
| CHP2_APC | >chr5: | AGCCCCAGTGATCTTCCAGATA | CCCTCTGAACTGCAGCATTT | 181 |
| _5 | 112175546 + | (SEQ ID NO: 28) | ACT (SEQ ID NO: 29) | |
| | 112175726 | | | |
| CHP2_APC | >chr5: | AGAGGGTCCAGGTTCTTCCA | TCATTTTCCTGAACTGGAG | 166 |
| _6 | 112175721 + | (SEQ ID NO: 30) | GCATT | |
| | 112175886 | | (SEQ ID NO: 31) | |
| CHP2_APC | >chr5: | ATGAAACAGAATCAGAGCAGC | CGTGATGACTTTGTTGGCA | 163 |
| _7 | 112175895 + | CTAAA (SEQ ID NO: 32) | TGG (SEQ ID NO: 33) | |
| | 112176057 | | | |
| CHP2_ATM | >chr11: | ATGATCTGCTAGTGAATGAGAT | ACAGGAAATTTCTAAATGT | 159 |
| _1 | 108117736 | AAGTCATA | GACATGACCT | |
| | 108117894 | (SEQ ID NO: 34) | (SEQ ID NO: 35) | |
| CHP2_ATM | >chr11: | AGTTCTGTTAAAGTTCATGGCT | AGCGTTTACGATCCTCTTTC | 130 |
| _10 | 108200888 | TTTGTG (SEQ ID NO: 36) | AGTG (SEQ ID NO: 37) | |
| | 108201017 | | | |
| CHP2_ATM | >chr11: | ACAGGAGAATATGGAAATCTG | TCACCTTAACAAGCTGTCTC | 136 |
| _12 | 108205705 | GTGACT | CTCT | |
| | 108205840 | (SEQ ID NO: 38) | (SEQ ID NO: 39) | |
| CHP2_ATM | >chr11: | TGCTATTCTCAGATGACTCTGT | TTCCGTGTTTCTCTGCAGTA | 157 |
| _13 | 108206496 | GTTTTT (SEQ ID NO: 40) | ATGT (SEQ ID NO: 41) | |
| | 108206652 | | | |
| CHP2_ATM | >chr11: | GTTTGTTTCTTTTTTCTCCAGTT | TCTTGGTAGGCAAACAACA | 184 |
| _14 | 108217985 | GGTTACAT | TTCCA | |
| | 108218168 | (SEQ ID NO: 42) | (SEQ ID NO: 43) | |
| CHP2_ATM | >chr11: | GTGTTTGACTCTAGATGCTGTG | TGAGATACACAGTCTACCT | 138 |
| _15 | 108225524 | AGAA (SEQ ID NO: 44) | GGTAAGAAAA | |
| | 108225661 | | (SEQ ID NO: 45) | |
| CHP2_ATM | >chr11: | CACCTCACTGAAACCTTTGTGT | AATTCACTTGTCCACCAACA | 147 |
| _16 | 108236018 | TTT (SEQ ID NO: 46) | CTGA (SEQ ID NO: 47) | |
| | 108236164 | | | |
| CHP2_ATM | >chr11: | GCTCATACAGCAGGCCATAGA | GTATGTTGGCAGGTTAAAA | 148 |
| _17 | 108236166 | (SEQ ID NO: 48) | ATAAAGGCT | |
| | 108236313 | | (SEQ ID NO: 49) | |
| CHP2_ATM | >chr11: | TCACCTTCAGAAGTCACAGAAT | TTGAGATGAAAGGATTCCA | 132 |
| _2 | 108119788 | GATTTT (SEQ ID NO: 50) | CTGAAAGTT | |
| | 108119919 | | (SEQ ID NO: 51) | |
| CHP2_ATM | >chr11: | AGTATTCTTTACATGGCTTTTG | CATTCTGGCACGCTTTGGA | 153 |
| _3 | 108123487 | GTCTTCT | AA (SEQ ID NO: 53) | |
| | 108123639 | (SEQ ID NO: 52) | | |
| CHP2_ATM | >chr11: | AAAAAGCCATTTGACCGTGGA | CAGGTTCGTTTGCATCACT | 139 |
| _4 | 108137910 | G (SEQ ID NO: 54) | AACA (SEQ ID NO: 55) | |
| | 108138048 | | | |
| CHP2_ATM | >chr11: | ACAGACTGCTTTCCAAAGATTC | GCCATACCTGTTTTCCCAAT | 151 |
| _5 | 108155057 | TTGTA (SEQ ID NO: 56) | AAGTTTT | |
| | 108155207 | | (SEQ ID NO: 57) | |
| CHP2_ATM | >chr11: | CTATATGTAGAGGCTGTTGGAA | TCAGCATTATGAAGGTCCA | 151 |
| _6 | 108170431 | GCTG (SEQ ID NO: 58) | CTGAAG | |
| | 108170581 | | (SEQ ID NO: 59) | |
| CHP2_ATM | >chr11: | ACTGGTGTACTTGATAGGCATT | TGTAGATAGGCCAGCATTG | 156 |
| _7 | 108172336 | TGAAT (SEQ ID NO: 60) | GATCT | |
| | 108172491 | | (SEQ ID NO: 61) | |
| CHP2 _ATM | >chr11: | AAACCCTTTTGAAGGCCTGGAT | TCACATTTTGTGCCTCCACT | 117 |
| _8 | 108173609 + | (SEQ ID NO: 62) | GT (SEQ ID NO: 63) | |
| | 108173725 | | | |
| CHP2_ATM | >chr11: | TCATTTTTCTTTAGACCTTCTTC | TGTAAAGTGAGCAGCACAA | 111 |
| _9 | 108180873 | AGGAACA (SEQ ID NO: 64) | GACT | |
| | 108180983 | | (SEQ ID NO: 65) | |
| CHP2_BRA | >chr7: | CATACTTACCATGCCACTTTCCC | TTTCTTTTTCTGTTTGGCTT | 176 |
| F_1 | 140481367 + | TT (SEQ ID NO: 66) | GACTTGA | |
| | 140481542 | | (SEQ ID NO: 67) | |
| CHP2_BRA | >chr7: | CCACAAAATGGATCCAGACAAC | GCTTGCTCTGATAGGAAAA | 172 |
| F_2 | 140453078 + | TGT (SEQ ID NO: 68) | TGAGATCTA | |
| | 140453249 | | (SEQ ID NO: 69) | |
| CHP2 _CDH | >chr16: | AGATTGCACCGGTCGACAAA | CCCAGGCGTAGACCAAGA | 137 |
| 1_1 | 68835583 + | (SEQ ID NO: 70) | AATG | |
| | 68835719 | | (SEQ ID NO: 71) | |
| CHP2_CDH | >chr16: | TGGTCCTGACTTGGTTGTGTC | GTTATAGAATTACCGTGGT | 176 |
| 1_2 | 68846004 + | (SEQ ID NO: 72) | GGGATTGAA | |
| | 68846179 | | (SEQ ID NO: 73) | |
| CHP2_CDH | >chr16: | GCAGTCTTGGTACTTTGTAAAT | TCAATATGGTGTATACAGC | 156 |
| 1_3 | 68847172 + | GACACA (SEQ ID NO: 74) | CTCCCA | |
| | 68847327 | | (SEQ ID NO: 75) | |
| CHP2_CSF1 | >chr5: | TCCACCATGACTTTGAGGTTGA | TCCCACTAATGCCAGATGC | 127 |
| R_1 | 149452969 + | G (SEQ ID NO: 76) | TTG (SEQ ID NO: 77) | |
| | 149453095 | | | |
| CHP2_CSF1 | >chr5: | CCATCCATGGAGGAGTTGAAG | CTCTAGTGAGCACCTGACC | 143 |
| R_2 | 149433571 + | TT (SEQ ID NO: 78) | TG (SEQ ID NO: 79) | |
| | 149433713 | | | |
| CHP2_CTN | >chr3: | ACTGTTTCGTATTTATAGCTGAT | CCTCTTCCTCAGGATTGCCT | 171 |
| NB1_1 | 41265999 + | TTGATGGA | TT (SEQ ID NO: 81) | |
| | 41266169 | (SEQ ID NO: 80) | | |
| CHP2_EGF | >chr7: | CCTCATTGCCCTCAACACAGT | TCAGTCCGGTTTTATTTGCA | 131 |
| R_1 | 55211024 + | (SEQ ID NO: 82) | TCATAGTT | |
| | 55211154 | | (SEQ ID NO: 83) | |
| CHP2_EGF | >chr7: | CACCACGTACCAGATGGATGT | CCCAAAGACTCTCCAAGAT | 173 |
| R_2 | 55221772 + | (SEQ ID NO: 84) | GGGATA | |
| | 55221944 | | (SEQ ID NO: 85) | |
| CHP2_EGF | >chr7: | AGACATGCATGAACATTTTTCT | TCCAGACCAGGGTGTTGTT | 139 |
| R_3 | 55232937 + | CCAC (SEQ ID NO: 86) | TTC (SEQ ID NO: 87) | |
| | 55233075 | | | |
| CHP2_EGF | >chr7: | TGTGGAGCCTCTTACACCCA | GTGCCAGGGACCTTACCTT | 137 |
| R_4 | 55241616 + | (SEQ ID NO: 88) | ATAC | |
| | 55241752 | | (SEQ ID NO: 89) | |
| CHP2_EGF | >chr7: | ACGTCTTCCTTCTCTCTCTGTCA | CTGAGGTTCAGAGCCATGG | 172 |
| R_5 | 55242389 + | (SEQ ID NO: 90) | A (SEQ ID NO: 91) | |
| | 55242560 | | | |
| CHP2_EGF | >chr7: | CATGCGAAGCCACACTGAC | CGGACATAGTCCAGGAGG | 164 |
| R_6 | 55248947 + | (SEQ ID NO: 92) | CA (SEQ ID NO: 93) | |
| | 55249110 | | | |
| CHP2_EGF | >chr7: | GACTATGTCCGGGAACACAAA | CCCCATGGCAAACTCTTGC | 167 |
| R_7 | 55249100 + | GA (SEQ ID NO: 94) | TA (SEQ ID NO: 95) | |
| | 55249266 | | | |
| CHP2_EGF | >chr7: | CGCAGCATGTCAAGATCACAG | GCATGTGTTAAACAATACA | 170 |
| R_8 | 55259485 + | AT (SEQ ID NO: 96) | GCTAGTG | |
| | 55259654 | | (SEQ ID NO: 97) | |
| CHP2_ERB | >chr17: | GAATGTGAAAATTCCAGTGGC | GTCATATCTCCCCAAACCCC | 176 |
| B2_1 | 37880188 + | CATC (SEQ ID NO: 98) | AAT (SEQ ID NO: 99) | |
| | 37880363 | | | |
| CHP2_ERB | >chr17: | GGGTGTGTGGTCTCCCATAC | GCCATAGGGCATAAGCTGT | 150 |
| B2_2 | 37880934 + | (SEQ ID NO: 100) | GTC | |
| | 37881083 | | (SEQ ID NO: 101) | |
| CHP2_ERB | >chr17: | GGATGAGCTACCTGGAGGATG | CCTTGGTCCTTCACCTAACC | 174 |
| B2_3 | 37881303 + | T (SEQ ID NO: 102) | TTG (SEQ ID NO: 103) | |
| | 37881476 | | | |
| CHP2_ERB | >chr2: | GCCTTAGAGTGTTCCTCAATGT | GAAACTTTGGACTTCAAGA | 148 |
| B4_1 | 212812049 + | AACAA | ACTTGGAT | |
| | 212812196 | (SEQ ID NO: 104) | (SEQ ID NO: 105) | |
| CHP2_ERB | >chr2: | CATCGCCACATAGGGTAGAAC | CAGACACCATTCATTGGCA | 139 |
| B4_2 | 212652695 + | ATTT (SEQ ID NO: 106) | AGATATTG | |
| | 212652833 | | (SEQ ID NO: 107) | |
| CHP2_ERB | >chr2: | CCTGAATCAAATAGGGAAGGA | GGCAGATGCTACGGACCTT | 149 |
| B4_3 | 212589739 + | AAGGA | A (SEQ ID NO: 109) | |
| | 212589887 | (SEQ ID NO: 108) | | |
| CHP2_ERB | >chr2: | TCTGTTACTTACGTGGACATTTC | CAGGCCTGCATGAATTTCA | 157 |
| B4_4 | 212587106 + | TTGAC | ATGA | |
| | 212587262 | (SEQ ID NO: 110) | (SEQ ID NO: 111) | |
| CHP2_ERB | >chr2: | GGCAAATGTCAGTGCAAGGTT | TGTTTTGAGCTTGTTTGCTG | 175 |
| B4_5 | 212578266 + | TA (SEQ ID NO: 112) | AATGT | |
| | 212578440 | | (SEQ ID NO: 113) | |
| CHP2_ERB | >chr2: | ACCCATGAATACCAGTGACTAG | CTCAATCCCCTAACTCTGAG | 163 |
| B4_6 | 212576773 + | AAAGA (SEQ ID NO: 114) | TCTTG | |
| | 212576935 | | (SEQ ID NO: 115) | |
| CHP2_ERB | >chr2: | GCCAGCAAGAATGCTTACCCTT | GGGTCCTGACAACTGTACA | 174 |
| B4_7 | 212530030 + | (SEQ ID NO: 116) | AAGT | |
| | 212530203 | | (SEQ ID NO: 117) | |
| CHP2_ERB | >chr2: | CATTTGACCATGACCATGTAAA | GGAACTGATGACCTTTGGA | 135 |
| B4_8 | 212288879 + | CGTC (SEQ ID NO: 118) | GGAA | |
| | 212289013 | | (SEQ ID NO: 119) | |
| CHP2_EZH | >chr7: | ACAATGCCACCTGAATACAGGT | GCATCTATTGCTGGCACCA | 133 |
| 2_1 | 18508681 + | TATC (SEQ ID NO: 120) | TCT | |
| | 148508813 | | (SEQ ID NO: 121) | |
| CHP2_FBX | >chr4: | TGACAATGTTTAAAGGTGGTA | ACTCATTGATAGTTGTGAA | 176 |
| W7_1 | 153258875 + | GCTGTT | CCAACACA | |
| | 153259050 | (SEQ ID NO: 122) | (SEQ ID NO: 123) | |
| CHP2_FBX | >chr4: | CCTGTGACTGCTGACCAAACTT | CACATCTTTCTTATAGGTGC | 126 |
| W7_2 | 153250828 + | TTA (SEQ ID NO: 124) | TGAAAGG | |
| | 153250953 | | (SEQ ID NO: 125) | |
| CHP2_FBX | >chr4: | CCCAACCATGACAAGATTTTCC | GGTCATCACAAATGAGAGA | 172 |
| W7_3 | 153249333 + | C (SEQ ID NO: 126) | CAACATCA | |
| | 153249504 | | (SEQ ID NO: 127) | |
| CHP2_FBX | >chr4: | ACTAACAACCCTCCTGCCATCA | TCTGCAGAGTTGTTAGCGG | 137 |
| W7_4 | 153247254 + | TA (SEQ ID NO: 128) | TT (SEQ ID NO: 129) | |
| | 153247390 | | | |
| CHP2_FBX | >chr4: | GTAGAATCTGCATTCCCAGAGA | TCTCTTGATACATCAATCCG | 134 |
| W7_5 | 153245386 + | CAA (SEQ ID NO: 130) | TGTTTGG | |
| | 153245519 | | (SEQ ID NO: 131) | |
| CHP2_FGF | >chr8: | ACCCAAAGGGCAGTAAGATAG | GGTCCCTAGGAGGAACCTC | 168 |
| R1_1 | 38285828 + | GAA (SEQ ID NO: 132) | A (SEQ ID NO: 133) | |
| | 38285995 | | | |
| CHP2_FGF | >chr8: | GGTCACTGTACACCTTACACAT | CCCTCTTTAGCCATGGCAA | 160 |
| R1_2 | 38282116 + | GAA (SEQ ID NO: 134) | GG (SEQ ID NO: 135) | |
| | 38282275 | | | |
| CHP2_FGF | >chr10: | CATCACTGTAAACCTTGCAGAC | TGGTCTCTCATTCTCCCATC | 154 |
| R2_1 | 123279582 | AAAC (SEQ ID NO: 136) | CC (SEQ ID NO: 137) | |
| | 123279735 | | | |
| CHP2_FGF | >chr10: | CATCCTCTCTCAACTCCAACAG | AGTGGATCAAGCACGTGG | 172 |
| R2_2 | 123279395 | G (SEQ ID NO: 138) | AAAA | |
| | 123279566 | | (SEQ ID NO: 139) | |
| CHP2_FGF | >chr10: | GCTTCTTGGTCGTGTTCTTCATT | CTCCTCCTGTGATCTGCAAT | 159 |
| R2_3 | 123274699 + | (SEQ ID NO: 140) | CT (SEQ ID NO: 141) | |
| | 123274857 | | | |
| CHP2_FGF | >chr10: | TGGAAGCCCAGCCATTTCTAAA | GATGATGAAGATGATTGG | 143 |
| R2_4 | 123257931 + | (SEQ ID NO: 142) | GAAACACAAG | |
| | 123258073 | | (SEQ ID NO: 143) | |
| CHP2_FGF | >chr4: | GTGACCGAGGACAACGTGAT | GCGTCCTACTGGCATGACC | 136 |
| R3_3 | 1807814 + | (SEQ ID NO: 144) | (SEQ ID NO: 145) | |
| | 1807949 | | | |
| CHP2_FGF | >chr4: | CTCTGGGAGATCTTCACGCT | CCACTCACAGGTCGTGTGT | 127 |
| R3_4 | 1808292 + | (SEQ ID NO: 146) | (SEQ ID NO: 147) | |
| | 1808418 | | | |
| CHP2_FGF | >chr4: | CGCCTTTCGAGCAGTACTCC | GCTAGGGACCCCTCACATT | 166 |
| R3_5 | 1808862 + | (SEQ ID NO: 148) | GT (SEQ ID NO: 149) | |
| | 1809027 | | | |
| CHP2_FLT3 | >chr13: | CTTGGGAGACTTGTCTGAACAC | GTGAGCTTATTTCACACGTT | 141 |
| _1 | 28610071 + | T (SEQ ID NO: 150) | CTTTTCT | |
| | 28610211 | | (SEQ ID NO: 151) | |
| CHP2_FLT3 | >chr13: | GCACATTCCATTCTTACCAAACT | TGACTCATCATTTCATCTCT | 174 |
| _2 | 28608202 + | CTA (SEQ ID NO: 152) | GAAGCAA | |
| | 28608375 | | (SEQ ID NO: 153) | |
| CHP2_FLT3 | >chr13: | CAAACATCCTCTTTGTCATCAA | GAGGCACTCATGTCAGAAC | 154 |
| _3 | 28602249 + | GCTAC (SEQ ID NO: 154) | TCAA | |
| | 28602402 | | (SEQ ID NO: 155) | |
| CHP2_FLT3 | >chr13: | CGACACAACACAAAATAGCCGT | CCACGGGAAAGTGGTGAA | 134 |
| _4 | 28592552 + | ATAAAA | GATA | |
| | 28592685 | (SEQ ID NO: 156) | (SEQ ID NO: 157) | |
| CHP2_GNA | >chr19: | GGATTGCAGATTGGGCCTTG | ACATGATGGATGTCACGTT | 137 |
| 11_1 | 3118862 + | (SEQ ID NO: 158) | CTCAAA | |
| | 3118998 | | (SEQ ID NO: 159) | |
| CHP2_GNA | >chr9: | ATAATCCATTGCCTGTCTAAAG | TGTTAACCTTGCAGAATGG | 175 |
| Q_1 | 80409348 + | AACACT | TCGAT | |
| | 80409522 | (SEQ ID NO: 160) | (SEQ ID NO: 161) | |
| CHP2_GNA | >chr20: | TTGGTGAGATCCATTGACCTCA | TGAATGTCAAGAAACCATG | 162 |
| 5_1 | 57484371 + | ATTT (SEQ ID NO: 162) | ATCTCTGTT | |
| | 57484532 | | (SEQ ID NO: 163) | |
| CHP2_GNA | >chr20: | CCTCTGGAATAACCAGCTGTCC | TGATCCCTAACAACACAGA | 156 |
| 5_2 | 57484541 + | (SEQ ID NO: 164) | AGCAA | |
| | 57484696 | | (SEQ ID NO: 165) | |
| CHP2 _HNF | >chr12: | GATTGAAGAGCCCACAGGTGA | CTCCTCCTTGCTAGGGTTCT | 130 |
| 1A_1 | 121431351 + | (SEQ ID NO: 166) | T (SEQ ID NO: 167) | |
| | 121431480 | | | |
| CHP2 _HNF | >chr12: | TGTCCCCATCACAGGCACAGG | GGCCCGCTGTACGTGTCCA | 130 |
| 1A_2 | 121431990 + | (SEQ ID NO: 168) | T (SEQ ID NO: 169) | |
| | 121432119 | | | |
| CHP2_HRA | >chr11: | CGCCAGGCTCACCTCTATAGT | CTGAGGAGCGATGACGGA | 132 |
| 5_1 | 534200 + | (SEQ ID NO: 170) | ATATAA | |
| | 534332 | | (SEQ ID NO: 171) | |
| CHP2_HRA | >chr11: | GACTTGGTGTTGTTGATGGCAA | CTGCAGGATTCCTACCGGA | 161 |
| 5_2 | 533790 + | A (SEQ ID NO: 172) | A (SEQ ID NO: 173) | |
| | 533950 | | | |
| CHP2_IDH1 | >chr2: | CCAACATGACTTACTTGATCCC | ATCACCAAATGGCACCATA | 150 |
| _1 | 209113079 + | CAT (SEQ ID NO: 174) | CGA | |
| | 209113228 | | (SEQ ID NO: 175) | |
| CHP2_JAK2 | >chr9: | TGAAGCAGCAAGTATGATGAG | CTGACACCTAGCTGTGATC | 174 |
| _1 | 5073706 + | CAA (SEQ ID NO: 176) | CTG | |
| | 5073879 | | (SEQ ID NO: 177) | |
| CHP2_JAK3 | >chr19: | CTGATTGCATGCCAGTCCTC | GCAAGGATTTGGCCAGTGC | 133 |
| _1 | 17954115 + | (SEQ ID NO: 178) | TAT | |
| | 17954247 | | (SEQ ID NO: 179) | |
| CHP2_JAK3 | >chr19: | CACGAGATGCCGGTACGA | GTCTGTGAGCACAAAATTT | 130 |
| _2 | 17947969 + | (SEQ ID NO: 180) | GGGAT | |
| | 17948098 | | (SEQ ID NO: 181) | |
| CHP2_JAK3 | >chr19: | GATGTCAGTCTGCCCTTCTGT | ACTTAGCTTGGAAGCTGAC | 162 |
| _3 | 17945596 + | (SEQ ID NO: 182) | AAGT | |
| | 17945757 | | (SEQ ID NO: 183) | |
| CHP2_KDR | >chr4: | CCCCTATCTCTCAAGCAAACTTC | AACTATCTGTTGGAGAAAA | 173 |
| _1 | 55980215 + | A (SEQ ID NO: 184) | GCTTGTCTT | |
| | 55980387 | | (SEQ ID NO: 185) | |
| CHP2_KDR | >chr4: | ACTCCGGGTTACACCATCTATA | TGCTTTGGAAGTTCAGTCA | 135 |
| _2 | 55979547 + | GTTAAG | ACTCTTT | |
| | 55979681 | (SEQ ID NO: 186) | (SEQ ID NO: 187) | |
| CHP2_KDR | >chr4: | CACTTCTCCATTCTTCACAAGG | GGCTGCGTTGGAAGTTATT | 169 |
| _3 | 55972928 + | GTA (SEQ ID NO: 188) | TCTAAG | |
| | 55973096 | | (SEQ ID NO: 189) | |
| CHP2_KDR | >chr4: | AGGTTGACCACATTGAGATGG | AGGGACCCCAATTATTGAA | 153 |
| _4 | 55962422 + | TG (SEQ ID NO: 190) | GGAAATG | |
| | 55962574 | | (SEQ ID NO: 191) | |
| CHP2_KDR | >chr4: | GCACTAGCCAGTACCTTCCTCT | GAGCAATCCCTGTGGATCT | 128 |
| _5 | 55960955 + | (SEQ ID NO: 192) | GAAA | |
| | 55961082 | | (SEQ ID NO: 193) | |
| CHP2_KDR | >chr4: | AAGAGATTTCCCAAATGTTCCA | AGCATTCAGGAAGAAAGA | 139 |
| _6 | 55955054 + | CCA (SEQ ID NO: 194) | GGCATT | |
| | 55955192 | | (SEQ ID NO: 195) | |
| CHP2_KDR | >chr4: | GGGATGTTAGGCCATATACAG | GCATGGAAGAGGATTCTG | 133 |
| _7 | 55953750 + | TACCT | GACT | |
| | 55953882 | (SEQ ID NO: 196) | (SEQ ID NO: 197) | |
| CHP2_KDR | >chr4: | GGTGTCTGTGTCATCGGAGT | GGTGAGGGTAAAAAGCAA | 168 |
| _8 | 55946231 + | (SEQ ID NO: 198) | AAGAATTGT | |
| | 55946398 | | (SEQ ID NO: 199) | |
| CHP2_KDR | >chr4: | CTCTCATGTGATGTCCAGGAGT | CCGTGTACTCCAGTGAGGA | 165 |
| _9 | 55946065 + | TG (SEQ ID NO: 200) | AG (SEQ ID NO: 201) | |
| | 55946229 | | | |
| CHP2_KIT_ | >chr4: | CGCCAAGGAAGAAGATCATAC | TTTGACAAAGCCCGGATCA | 176 |
| 1 | 55561630 + | TCAA (SEQ ID NO: 202) | GT (SEQ ID NO: 203) | |
| | 55561805 | | | |
| CHP2_KIT_ | >chr4: | CCACACCCTGTTCACTCCTTT | GTCTCAGTCATTAGAGCAC | 141 |
| 3 | 55593397 + | (SEQ ID NO: 204) | TCTGG | |
| | 55593537 | | (SEQ ID NO: 205) | |
| CHP2_KIT_ | >chr4: | AAGGTGATCTATTTTTCCCTTTC | TTTCATACTGACCAAAACTC | 171 |
| 4 | 55593550 + | TCC (SEQ ID NO: 206) | AGCCT | |
| | 55593720 | | (SEQ ID NO: 207) | |
| CHP2_KIT_ | >chr4: | GCTTTTTGCTAAAATGCATGTTT | GACACGGCTTTACCTCCAA | 157 |
| 5 | 55594144 + | CCAA (SEQ ID NO: 208) | TG (SEQ ID NO: 209) | |
| | 55594300 | | | |
| CHP2_KIT_ | >chr4: | ACCTTCTTTCTAACCTTTTCT | CTGCTTTGAACAAATAAAT | 126 |
| 6 | 55595467 + | TATGTGCTT | GAATCACGTTT | |
| | 55595592 | (SEQ ID NO: 210) | (SEQ ID NO: 211) | |
| CHP2_KIT_ | >chr4: | AGGAGGTAGAGCATGACCCAT | GGGACAACATAAGAAACTC | 135 |
| 7 | 55597416 + | (SEQ ID NO: 212) | CAGGTTT | |
| | 55597550 | | (SEQ ID NO: 213) | |
| CHP2_KIT_ | >chr4: | CAGCCAGAAATATCCTCCTTAC | GTCAAGCAGAGAATGGGT | 128 |
| 8 | 55599255 + | TCAT (SEQ ID NO: 214) | ACTCAC | |
| | 55599382 | | (SEQ ID NO: 215) | |
| CHP2_KIT_ | >chr4: | GTGCTTCTATTACAGGCTCGAC | CCTAAAGAGAACAGCTCCC | 128 |
| 9 | 55602649 + | TAC (SEQ ID NO: 216) | AAAGAA | |
| | 55602776 | | (SEQ ID NO: 217) | |
| CHP2_KRA | >chr12: | CAAAGAATGGTCCTGCACCAGT | AGGCCTGCTGAAAATGACT | 172 |
| S_1 | 25398160 + | AATAT (SEQ ID NO: 218) | GAATATAA | |
| | 25398331 | | (SEQ ID NO: 219) | |
| CHP2_KRA | >chr12: | TCCTCATGTACTGGTCCCTCATT | GTAAAAGGTGCACTGTAAT | 156 |
| S_2 | 25380238 + | (SEQ ID NO: 220) | AATCCAGACT | |
| | 25380393 | | (SEQ ID NO: 221) | |
| CHP2_KRA | >chr12: | CAGATCTGTATTTATTTCAGTGT | GACTCTGAAGATGTACCTA | 168 |
| S_3 | 25378518 + | TACTTACCT | TGGTCCTA | |
| | 25378685 | (SEQ ID NO: 222) | (SEQ ID NO: 223) | |
| CHP2_MET | >chr7: | CTGACATACAGTCGGAGGTTCA | AGAAGTTGATGAACCGGTC | 132 |
| _1 | 116339593 + | C (SEQ ID NO: 224) | CTTT | |
| | 116339724 | | (SEQ ID NO: 225) | |
| CHP2_MET | >chr7: | CAAATAGGAGCCAGCCTGAAT | GGAGACATCTCACATTGTT | 166 |
| _2 | 116340132 + | GAT (SEQ ID NO: 226) | TTTGTTGA | |
| | 116340297 | | (SEQ ID NO: 227) | |
| CHP2_MET | >chr7: | CATGTCAACATCGCTCTAATTC | GCTTTTCAAAAGGCTTAAA | 174 |
| _3 | 116403105 + | AGAGA | CACAGGAT | |
| | 116403278 | (SEQ ID NO: 228) | (SEQ ID NO: 229) | |
| CHP2_MET | >chr7: | CCCATGATAGCCGTCTTTAACA | CGGTAGTCTACAGATTCAT | 172 |
| _4 | 116411855 + | AG (SEQ ID NO: 230) | TTGAAACCAT | |
| | 116412026 | | (SEQ ID NO: 231) | |
| CHP2_MET | >chr7: | GCTGATTTTGGTCTTGCCAGAG | TCTGACTTGGTGGTAAACT | 134 |
| _6 | 116423386 + | (SEQ ID NO: 232) | TTTGAGTT | |
| | 116423519 | | (SEQ ID NO: 233) | |
| CHP2_MLH | >chr3: | TCTGACCTCGTCTTCTACTTCTG | CCCTGCCACTAGAAATATC | 176 |
| 1_1 | 37067184 + | G (SEQ ID NO: 234) | TGTCTTA | |
| | 37067359 | | (SEQ ID NO: 235) | |
| CHP2_MPL | >chr1: | TGACCGCTCTGCATCTAGTG | AGCGAACCAAGAATGCCTG | 161 |
| _1 | 43814949 + | (SEQ ID NO: 236) | TTTA | |
| | 43815109 | | (SEQ ID NO: 237) | |
| CHP2_NOT | >chr9: | CACGCTTGAAGACCACGTTG | GGACTGTGCGGAGCATGT | 149 |
| CH1_1 | 139399318 + | (SEQ ID NO: 238) | A (SEQ ID NO: 239) | |
| | 139399466 | | | |
| CHP2_NOT | >chr9: | ACACACTGCCGGTTGTCAA | CCTCACCATGTCCTGACTGT | 157 |
| CH1_2 | 139397744 + | (SEQ ID NO: 240) | G (SEQ ID NO: 241) | |
| | 139397900 | | | |
| CHP2_NPM | >chr5: | GATGTCTATGAAGTGTTGTGGT | GACAGCCAGATATCAACTG | 168 |
| 1_1 | 170837476 + | TCCT (SEQ ID NO: 242) | TTACAGAA | |
| | 170837643 | | (SEQ ID NO: 243) | |
| CHP2_NRA | >chr1: | CCTCACCTCTATGGTGGGATCA | GTTCTTGCTGGTGTGAAAT | 134 |
| S_1 | 115258665 + | TAT (SEQ ID NO: 244) | GACTG | |
| | 115258798 | | (SEQ ID NO: 245) | |
| CHP2_NRA | >chr1: | TTCGCCTGTCCTCATGTATTGG | CACCCCCAGGATTCTTACA | 126 |
| S_2 | 115256483 + | (SEQ ID NO: 246) | GAAAA | |
| | 115256608 | | (SEQ ID NO: 247) | |
| CHP2_NRA | >chr1: | GCACAAATGCTGAAAGCTGTA | CAAGTGTGATTTGCCAACA | 130 |
| S_3 | 115252163 + | CC (SEQ ID NO: 248) | AGGA | |
| | 115252292 | | (SEQ ID NO: 249) | |
| CHP2_PDG | >chr4: | GCACTGGGACTTTGGTAATTCA | CATCTCTTGGAAACTCCCAT | 170 |
| FRA_1 | 55140959 + | C (SEQ ID NO: 250) | CTTGA | |
| | 55141128 | | (SEQ ID NO: 251) | |
| CHP2_PDG | >chr4: | CAGTGAAAAACAAGCTCTCATG | CCACATGTGTCCAGTGAAA | 140 |
| FRA_2 | 55144076 + | TCTG | ATCCT | |
| | 55144219 | (SEQ ID NO: 252) | (SEQ ID NO: 253) | |
| CHP2_PDG | >chr4: | TGTCCCCATAGGCCCCATTTA | TGCTTTCATCAGCAGGGTT | 158 |
| FRA_3 | 55144518 + | (SEQ ID NO: 254) | CAA | |
| | 55144675 | | (SEQ ID NO: 255) | |
| CHP2_PDG | >chr4: | CAGTGTGTCCACCGTGATCT | AGTGAAGGAGGATGAGCC | 171 |
| FRA_4 | 55152005 + | (SEQ ID NO: 256) | TGA | |
| | 55152175 | | (SEQ ID NO: 257) | |
| CHP2_PIK3 | >chr3: | CCATAAAGCATGAACTATTTAA | GGTTGAAAAAGCCGAAGG | 160 |
| CA_1 | 178916744 + | AGAAGCAAGA | TCAC | |
| | 178916903 | (SEQ ID NO: 258) | (SEQ ID NO: 259) | |
| CHP2_PIK3 | >chr3: | TGGAATGCCAGAACTACAATCT | GTGGAAGATCCAATCCATT | 157 |
| CA_10 | 178951969 + | TTTGAT | TTTGTTGTC | |
| | 178952125 | (SEQ ID NO: 260) | (SEQ ID NO: 261) | |
| CHP2_PIK3 | >chr3: | TGGATCTTCCACACAATTAAAC | TGCTGTTCATGGATTGTGC | 148 |
| CA_11 | 178952114 + | AGCAT (SEQ ID NO: 262) | AATTC | |
| | 178952261 | | (SEQ ID NO: 263) | |
| CHP2_PIK3 | >chr3: | GACGCATTTCCACAGCTACAC | AGCATCAGCATTTGACTTT | 155 |
| CA_3 | 178921444 + | (SEQ ID NO: 264) | ACCTTATCA | |
| | 178921598 | | (SEQ ID NO: 265) | |
| CHP2_PIK3 | >chr3: | CATAGGTGGAATGAATGGCTG | TCAATCAGCGGTATAATCA | 176 |
| CA_4 | 178927378 + | AATTATG | GGAGTTTTT | |
| | 178927553 | (SEQ ID NO: 266) | (SEQ ID NO: 267) | |
| CHP2_PIK3 | >chr3: | GCTTTGAATCTTTGGCCAGTAC | CATAAGAGAGAAGGTTTG | 142 |
| CA_6 | 178928046 + | CT (SEQ ID NO: 268) | ACTGCCATA | |
| | 178928187 | | (SEQ ID NO: 269) | |
| CHP2_PIK3 | >chr3: | CAGAGTAACAGACTAGCTAGA | GCACTTACCTGTGACTCCAT | 136 |
| CA_7 | 178935995 + | GACAATGA | AGAAA | |
| | 178936130 | (SEQ ID NO: 270) | (SEQ ID NO: 271) | |
| CHP2_PIK3 | >chr3: | GATGCAGCCATTGACCTGTTTA | AGAAAACCATTACTTGTCC | 127 |
| CA_9 | 178947796 + | C (SEQ ID NO: 272) | ATCGTCT | |
| | 178947922 | | (SEQ ID NO: 273) | |
| CHP2_PTE | >chr10: | GCCATCTCTCTCCTCCTTTTTCT | GCCGCAGAAATGGATACA | 139 |
| N_1 | 89624184 + | T (SEQ ID NO: 274) | GGTC | |
| | 89624322 | | (SEQ ID NO: 275) | |
| CHP2_PTE | >chr10: | TGTTAATGGTGGCTTTTTGTTT | TCTACCTCACTCTAACAAGC | 172 |
| N_2 | 89685231 + | GTTTGT | AGATAACT | |
| | 89685402 | (SEQ ID NO: 276) | (SEQ ID NO: 277) | |
| CHP2_PTE | >chr10: | CCATAACCCACCACAGCTAGAA | TGCCCCGATGTAATAAATA | 155 |
| N_3 | 89692792 + | (SEQ ID NO: 278) | TGCACAT | |
| | 89692946 | | (SEQ ID NO: 279) | |
| CHP2_PTE | >chr10: | GGCTACGACCCAGTTACCATAG | TGCCACTGGTCTATAATCC | 176 |
| N_4 | 89711783 + | (SEQ ID NO: 280) | AGATGAT | |
| | 89711958 | | (SEQ ID NO: 281) | |
| CHP2_PTE | >chr10: | TGAGATCAAGATTGCAGATAC | ACCTTTAGCTGGCAGACCA | 165 |
| N_5 | 89717476 + | AGAATCC | C (SEQ ID NO: 283) | |
| | 89717640 | (SEQ ID NO: 282) | | |
| CHP2_PTE | >chr10: | GCAGTATAGAGCGTGCAGATA | CATCACATACATACAAGTC | 168 |
| N_8 | 89720760 + | ATGA (SEQ ID NO: 284) | AACAACCC | |
| | 89720927 | | (SEQ ID NO: 285) | |
| CHP2_PTP | >chr12: | GCCTCCCTTTCCAATGGACTATT | CTTTTAATTGCCCGTGATGT | 158 |
| N11_1 | 112888095 | T (SEQ ID NO: 286) | TCCA | |
| | 112888252 | | (SEQ ID NO: 287) | |
| CHP2_PTP | >chr12: | TGATGTTTCCTTCGTAGGTGTT | TGGTACCTGCTCTTCTTCAA | 175 |
| N11_2 | 112926811 | GAC (SEQ ID NO: 288) | TCCT | |
| | 112926985 | | (SEQ ID NO: 289) | |
| CHP2_RB1 | >chr13: | ACTTTTTTCTATTCTTTCCT | CCTTTCCAATTTGCTGAAGA | 147 |
| _1 | 48919190 + | TTGTAGTGTCCATA | GTGC | |
| | 48919336 | (SEQ ID NO: 290) | (SEQ ID NO: 291) | |
| CHP2_RB1 | >chr13: | TCTTCCTCAGACATTCAAACGT | ACCTACCCTGGTGGAAGCA | 130 |
| _10 | 49039124 + | GTTT (SEQ ID NO: 292) | TA (SEQ ID NO: 293) | |
| | 49039253 | | | |
| CHP2_RB1 | >chr13: | GCATTGGTGCTAAAAGTTTCTT | AAGCAGAGAATGAGGGAG | 165 |
| _2 | 48923114 + | GGAT (SEQ ID NO: 294) | GAGTA | |
| | 48923278 | | (SEQ ID NO: 295) | |
| CHP2_RB1 | >chr13: | GCTGAGAGATGTAATGACATG | CCATGTGCAATACCTGTCT | 179 |
| _3 | 48941574 + | TAAAGGA | ATAGAATCA | |
| | 48941752 | (SEQ ID NO: 296) | (SEQ ID NO: 297) | |
| CHP2_RB1 | >chr13: | TGAGACAACAGAAGCATTATA | CTGGAGTGTGTGGAGGAA | 167 |
| _4 | 48942570 + | CTGCTTT | TTACATT | |
| | 48942736 | (SEQ ID NO: 298) | (SEQ ID NO: 299) | |
| CHP2_RB1 | >chr13: | AGAAGGCAACTTGACAAGAGA | CAATAATTTGTTAGCCATAT | 140 |
| _6 | 48955498 + | AATGATA | GCACATGAATGA | |
| | 48955637 | (SEQ ID NO: 300) | (SEQ ID NO: 301) | |
| CHP2_RB1 | >chr13: | CTGGGAAAATTATGCTTACTAA | ACAAGCAGATTCAAGGTGA | 128 |
| _7 | 49027076 + | TGTGGTTT | TCAGTT | |
| | 49027203 | (SEQ ID NO: 302) | (SEQ ID NO: 303) | |
| CHP2_RB1 | >chr13: | AGTAAAAATGACTAATTTTTCT | TGCCTGTCTCTCATGAGTTC | 169 |
| _8 | 49033791 + | TATTCCCACAGTGTA | ATACT | |
| | 49033959 | (SEQ ID NO: 304) | (SEQ ID NO: 305) | |
| CHP2_RB1 | >chr13: | AACAAAACCATGTAATAAAATT | GAGGAAGATCCTTGTATGC | 144 |
| _9 | 49037814 + | CTGACTACTTT | TGTTAC | |
| | 49037957 | (SEQ ID NO: 306) | (SEQ ID NO: 307) | |
| CHP2_RET_ | >chr10: | GGGATTAAAGCTGGCTATGGC | CCTTGTTGGGACCTCAGAT | 159 |
| 1 | 43609045 + | A (SEQ ID NO: 308) | GT (SEQ ID NO: 309) | |
| | 43609203 | | | |
| CHP2_RET_ | >chr10: | AGCATACGCAGCCTGTACC | GTGGTAGCAGTGGATGCA | 176 |
| 2 | 43609856 + | (SEQ ID NO: 310) | GAA | |
| | 43610031 | | (SEQ ID NO: 311) | |
| CHP2_RET_ | >chr10: | GCTTCCAGGAGCGATCGTTT | AGGCCCCATACAATTTGAT | 142 |
| 3 | 43613775 + | (SEQ ID NO: 312) | GACA | |
| | 43613916 | | (SEQ ID NO: 313) | |
| CHP2_RET_ | >chr10: | CTGGTTACTGAAAGCTCAGGG | ACTTTGCGTGGTGTAGATA | 168 |
| 5 | 43617292 + | AT (SEQ ID NO: 314) | TGATCAA | |
| | 43617459 | | (SEQ ID NO: 315) | |
| CHP2_SMA | >chr18: | CTCATGTGATCTATGCCCGTCT | AGTCTACTTACCAATTCCAG | 164 |
| D4_1 | 48575078 + | (SEQ ID NO: 316) | GTGATACA | |
| | 48575241 | | (SEQ ID NO: 317) | |
| CHP2_SMA | >chr18: | TGCTACTTCTGAATTGAAATGG | GATTACCTACCATTACTCTG | 174 |
| D4_2 | 48575531 + | TTCA (SEQ ID NO: 318) | CAGTGTT | |
| | 48575704 | | (SEQ ID NO: 319) | |
| CHP2_SMA | >chr18: | ATGGTGAAGGATGAATATGTG | GCTGGTAGCATTAGACTCA | 162 |
| D4_3 | 48581165 + | CATGA | GATGG | |
| | 48581326 | (SEQ ID NO: 320) | (SEQ ID NO: 321) | |
| CHP2_SMA | >chr18: | GTGAAGGACTGTTGCAGATAG | AAGGCCCACATGGGTTAAT | 173 |
| D4_4 | 48584528 + | CAT (SEQ ID NO: 322) | TTG | |
| | 48584700 | | (SEQ ID NO: 323) | |
| CHP2_SMA | >chr18: | TTTCTTTAGGGCCTGTTCACAAT | CTGAGAAGTGACCCCATAA | 161 |
| D4_5 | 48586227 + | GA (SEQ ID NO: 324) | TTCCATT | |
| | 48586387 | | (SEQ ID NO: 325) | |
| CHP2_SMA | >chr18: | GCTCCTGAGTATTGGTGTTCCA | CCTGTGGACATTGGAGAGT | 162 |
| D4_6 | 48591792 + | T (SEQ ID NO: 326) | TGA | |
| | 48591953 | | (SEQ ID NO: 327) | |
| CHP2_SMA | >chr18: | TGTAATTTCTTTTTTCTTCCTAA | ACTTGGGTAGATCTTATGA | 181 |
| D4_7 | 48593365 + | GGTTGCACATAG | ACAGCAT | |
| | 48593545 | (SEQ ID NO: 328) | (SEQ ID NO: 329) | |
| CHP2_SMA | >chr18: | AGGTCTTTGATTTGCGTCAGTG | GCTGGAGCTATTCCACCTA | 136 |
| D4_8 | 48603006 + | T (SEQ ID NO: 330) | CTG | |
| | 48603141 | | (SEQ ID NO: 331) | |
| CHP2_SMA | >chr18: | GCTGCTGGAATTGGTGTTGATG | AGTACTTCGTCTAGGAGCT | 161 |
| D4_9 | 48604637 + | (SEQ ID NO: 332) | GGAG | |
| | 48604797 | | (SEQ ID NO: 333) | |
| CHP2_SMA | >chr22: | CTTGCTTTACTCATAGGTGGGA | ACGCACCCTTAGTGTTAGG | 161 |
| RCB1_1 | 24133927 + | AACTA (SEQ ID NO: 334) | TTTT | |
| | 24134087 | | (SEQ ID NO: 335) | |
| CHP2_SMA | >chr22: | GCTCCCACCACTTAGATGCC | AACTGAAACGTGCTGGAG | 155 |
| RCB1_2 | 24143181 + | (SEQ ID NO: 336) | AACTAA | |
| | 24143335 | | (SEQ ID NO: 337) | |
| CHP2_SMA | >chr22: | CTGACTGTTGCTTCCATTTCACT | GACTGCCTTGTACCATTCAT | 169 |
| RCB1_3 | 24145454 + | T (SEQ ID NO: 338) | GTTC | |
| | 24145622 | | (SEQ ID NO: 339) | |
| CHP2_SMA | >chr22: | CACTTGGCTGCCCTGTAGAG | CCAATCTTCTGAGATGCTCC | 174 |
| RCB1_4 | 24176240 + | (SEQ ID NO: 340) | GT (SEQ ID NO: 341) | |
| | 24176413 | | | |
| CHP2_SMO | >chr7: | CCAGAATGAGGTGCAGAACAT | CGATGTAGCTGTGCATGTC | 170 |
| _1 | 128845040 + | CAA (SEQ ID NO: 342) | CT (SEQ ID NO: 343) | |
| | 128845209 | | | |
| CHP2_SMO | >chr7: | CAGGTAGAGGGAGTACAGAGT | GGCATAGGTGAGGACCAC | 150 |
| _2 | 128845935 + | GA (SEQ ID NO: 344) | AAA | |
| | 128846084 | | (SEQ ID NO: 345) | |
| CHP2_SMO | >chr7: | GGACTCTGTGAGTGGGATTTGT | GTCTTCACTCACCTCGGAT | 128 |
| _3 | 128846313 + | TTT (SEQ ID NO: 346) | GA (SEQ ID NO: 347) | |
| | 128846440 | | | |
| CHP2_SMO | >chr7: | CATCCCTGACTGTGAGATCAAG | CAGGTACGCCTCCAGATGA | 138 |
| _4 | 128850246 + | AA (SEQ ID NO: 348) | G (SEQ ID NO: 349) | |
| | 128850383 | | | |
| CHP2_SMO | >chr7: | GGCTTGGCCTTTGACCTCAAT | TCCTCCAGAAGCTTGAACT | 159 |
| _5 | 128851479 + | (SEQ ID NO: 350) | CTCATA | |
| | 128851637 | | (SEQ ID NO: 351) | |
| CHP2_STK1 | >chr19: | AACATCACCACGGGTCTGTAC | GATGAGGCTCCCACCTTTC | 138 |
| 1_4 | 1221216 + | (SEQ ID NO: 352) | AG (SEQ ID NO: 353) | |
| | 1221353 | | | |
| CHP2_STK1 | >chr19: | GAAGAAACATCCTCCGGCTGA | ACCGTGAAGTCCTGAGTGT | 174 |
| 1_5 | 1222993 + | A (SEQ ID NO: 354) | AGA | |
| | 1223166 | | (SEQ ID NO: 355) | |
| CHP2_TP53 | >chr17: | TCCACTCACAGTTTCCATAGGT | GTTGGAAGTGTCTCATGCT | 154 |
| _1 | 7579830 + | CT (SEQ ID NO: 356) | GGAT | |
| | 7579983 | | (SEQ ID NO: 357) | |
| CHP2_TP53 | >chr17: | GGCTGTCCCAGAATGCAAGAA | GATGAAGCTCCCAGAATGC | 177 |
| _2 | 7579330 + | (SEQ ID NO: 358) | CA (SEQ ID NO: 359) | |
| | 7579506 | | | |
| CHP2_TP53 | >chr17: | CCAGTTGCAAACCAGACCTCA | AGGCCTCTGATTCCTCACT | 161 |
| _5 | 7578160 + | (SEQ ID NO: 360) | GAT | |
| | 7578320 | | (SEQ ID NO: 361) | |
| CHP2_TP53 | >chr17: | GGCTCCTGACCTGGAGTCTT | CTCATCTTGGGCCTGTGTTA | 148 |
| _6 | 7577489 + | (SEQ ID NO: 362) | TCTC | |
| | 7577636 | | (SEQ ID NO: 363) | |
| CHP2_TP53 | >chr17: | CGCTTCTTGTCCTGCTTGCT | TTCTCTTTTCCTATCCTGAG | 183 |
| _7 | 7576996 + | (SEQ ID NO: 364) | TAGTGGT | |
| | 7577178 | | (SEQ ID NO: 365) | |
| CHP2_VHL | >chr3: | CTCCCAGGTCATCTTCTGCAAT | GTACCTCGGTAGCTGTGGA | 132 |
| _1 | 10183744 + | (SEQ ID NO: 366) | TG (SEQ ID NO: 367) | |
| | 10183875 | | | |
| CHP2_VHL | >chr3: | GTGGCTCTTTAACAACCTTTGC | GTCAGTACCTGGCAGTGTG | 165 |
| _2 | 10188164 + | T (SEQ ID NO: 368) | ATA | |
| | 10188328 | | (SEQ ID NO: 369) | |
| CHP2_VHL | >chr3: | GGCAAAGCCTCTTGTTCGTTC | TGACGATGTCCAGTCTCCT | 154 |
| _3 | 10191398 + | (SEQ ID NO: 370) | GTAAT | |
| | 10191551 | | (SEQ ID NO: 371) | |

What is claimed is:

1. A method for sequencing at least one amplicon, comprising the steps of:
a) providing at least one amplicon, wherein the at least one amplicon comprises a sequence of interest and a preceding sequence 5′ to the sequence of interest incorporated from a first priming sequence;
b) amplifying the at least one amplicon in a first reaction mixture comprising a plurality of nuclease-sensitive amplification primers to form an amplified DNA product;
c) contacting the first reaction mixture containing the amplified DNA product with a second reaction mixture comprising a nuclease and at least one chemically-enhanced primer whereby the plurality of nuclease sensitive amplification primers are degraded by the nuclease;
d) inactivating the nuclease;
e) priming the amplified DNA product with the at least one chemically-enhanced primer in a sequencing reaction; and
f) producing extension products of the at least one chemically enhanced primer.

2. The method of claim 1, wherein the extension products are fluorescently labeled.

3. The method of any one of the preceding claims, wherein the first priming sequence was used to produce the amplicon.

4. The method of any one of the preceding claims, wherein the first priming sequence comprises at least one cleavable moiety.

5. The method of claim 4, wherein the preceding sequence is a portion of the first priming sequence.

6. The method of any one of the preceding claims, wherein the steps of contacting the first reaction mixture with the second reaction mixture, inactivating the nuclease, and producing the extension products of the chemically enhanced primer are performed in the same reaction vessel.

7. The method of any one of the preceding claims, wherein the steps of amplifying the at least one amplicon, contacting the first reaction mixture with the second reaction mixture, inactivating the nuclease, and producing the extension products of the chemically enhanced primer are performed without intermediate purification steps.

8. The method of any one of the preceding claims, wherein the at least one amplicon further comprises a succeeding sequence 3′ to the sequence of interest wherein the succeeding sequence is complementary to a second priming sequence used to produce the at least one amplicon.

9. The method of any one of the preceding claims, wherein the at least one amplicon has a length of about 100 nucleotides to about 400 nucleotides.

10. The method of any one of the preceding claims, wherein the sequence of interest has a length of about 100 nucleotides to about 300 nucleotides.

11. The method of any one of the preceding claims, wherein the at least one amplicon is a plurality of amplicons.

12. The method of claim 11, wherein the plurality of amplicons comprise at least two different amplicons, a first having a sequence of interest comprising a major variant sequence and a second comprising a minor variant sequence from the same region of a sample nucleic acid.

13. The method of any one of the preceding claims, further comprising:
a) obtaining sequencing results based on the sequencing reaction; and
b) determining a nucleotide base sequence of at least the sequence of interest based on the results.

14. The method of claim 13, wherein the sequencing results are obtained via a mobility based separation method.

15. The method of claim 14, wherein the mobility based separation method is capillary electrophoresis.

16. The method of claim 13, wherein the determined nucleotide base sequence of at least the sequence of interest is compared to a second nucleotide base sequence of at least the sequence of interest obtained from a NGS method of sequencing.

17. The method of claim 16, wherein the NGS method of sequencing is semiconductor sequencing.

18. The method of any one of the preceding claims, wherein the second reaction mixture further comprises a polymerase, deoxynucleotide triphosphates, and dye-labelled dideoxynucleotide triphosphates.

19. The method of any one of the preceding claims, wherein amplifying DNA comprises polymerase chain reaction amplification.

20. The method of any one of the preceding claims, wherein the first reaction mixture further comprises a polymerase.

21. The method of claim 20, wherein the polymerase is a thermostable polymerase.

22. The method of any one of claims 20-21, wherein the polymerase is Taq polymerase.

23. The method of any one of the preceding claims, wherein the polymerase of the second reaction mixture is a thermostable polymerase.

24. The method of claim 23, wherein the polymerase is Taq polymerase

25. The method of any one of the preceding claims, wherein the nuclease is selected from exonuclease I, Exo III, Pfu and DNA pol I.

26. The method of any one of the preceding claims, wherein the chemically-enhanced primer comprises an oligonucleotide sequence, a NCM and none or at least one nuclease-resistant linkage.

27. The method of any one of the preceding claims, wherein the chemically-enhanced primer comprises one nuclease-resistant linkage at a terminal 3′ end.

28. The method of any one of the preceding claims, wherein the sequencing reaction comprises cycle sequencing.

29. The method of any one of the preceding claims, wherein the chemically-enhanced primer comprises a plurality of NCMs either at a terminal 5′ end or within a oligonucleotide sequence of the chemically-enhanced primer.

30. The method of any one of the preceding claims, wherein the NCM is a (Cn) spacer wherein n is any integer from 1 to 9.

31. The method of claim 30, wherein the NCM comprises a plurality of (Cn) spacers.

32. The method of any one of the preceding claims, wherein the chemically-enhanced primer has a structure of the formula:
(Cn)ₓ-OLIGO, wherein (Cn)ₓ has a structure of the following formula: 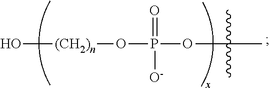
wherein each instance of n is independently an integer of 1 to 9; and x is an integer of 1 to about 30;
OLIGO has a structure of the following formula: 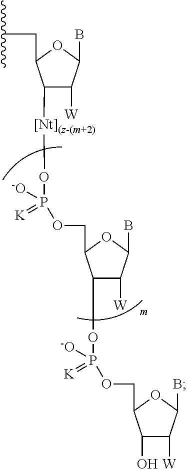
wherein B is a nucleobase;
K is S or O;
m is 0 or 1;
z is an integer of 3 to about 100;
W is OH, F, OMe, or H; and
Nt is a moiety having a formula:

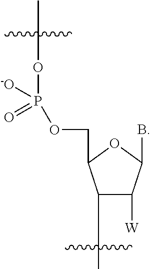

33. The method of any one of the preceding claims, wherein each of the plurality of nuclease-sensitive amplification primers is configured to prime a sequence of interest of a specific disease state.

34. The method of any one of the preceding claims, wherein the plurality of nuclease-sensitive amplification primers prime a set of sequences connected to a specific disease state.

35. A method for confirming a DNA sequence, comprising the steps of:
a) amplifying a sample comprising nucleic acid using at least a first priming sequence to provide a plurality of amplicons, wherein each of the plurality of amplicons comprises a sequence of interest and a preceding sequence 5′ to the sequence of interest incorporated from a first priming sequence;
b) amplifying a first aliquot of the plurality of amplicons in a first reaction mixture comprising a plurality of nuclease-sensitive amplification primers to form an amplified DNA product;
c) contacting the first reaction mixture containing the amplified DNA product with a second reaction mixture comprising a nuclease and at least one chemically-enhanced primer whereby the nuclease sensitive amplification primers are degraded by the nuclease;
d) inactivating the nuclease;
e) priming the amplified DNA product with the at least one chemically-enhanced primer in a sequencing reaction; and
f) producing extension products of the chemically enhanced primer.

36. The method of claim 35, wherein the extension products are fluorescently labeled.

37. The method of any one of claims 35-36, wherein the first priming sequence was used to produce the amplicon.

38. The method of any one of claims 35-37, wherein the first priming sequence comprises at least one cleavable moiety.

39. The method of claim 38, wherein the steps of contacting the first reaction mixture with the second reaction mixture, inactivating the nuclease, and producing the extension products of the chemically enhanced primer are performed in the same reaction vessel.

40. The method of any one of claims 35-39, wherein the steps of amplifying the plurality of amplicons, contacting the first reaction mixture with the second reaction mixture, inactivating the nuclease, and producing the extension products of the chemically enhanced primer are performed without intermediate purification steps.

41. The method of any one of claims 35-40, wherein each of the plurality of amplicons further comprises a succeeding sequence 3′ to the sequence of interest wherein the succeeding sequence is complementary to a second priming sequence used to produce the at least one amplicon.

42. The method of any one of claims 35-41, wherein the plurality of amplicons comprise at least two different amplicons a first having a sequence of interest comprising a major variant sequence and a second comprising a minor variant sequence from the same region of a sample nucleic acid.

43. The method of any one of claims 35-42, wherein each of the plurality of amplicons has a length of about 100 nucleotides to about 400 nucleotides.

44. The method of any one of claims 35-43, wherein each sequence of interest of each of the plurality of amplicons has a length of about 100 nucleotides to about 300 nucleotides.

45. The method of any one of claims 35-44, further comprising:
a) obtaining sequencing results based on the sequencing reaction; and
b) determining a nucleotide base sequence of at least the sequence of interest based on the results.

46. The method of claim 45, wherein the sequencing results are obtained via a mobility based separation method.

47. The method of claim 46, wherein the mobility based separation method is capillary electrophoresis.

48. The method of claim 45, wherein the determined nucleotide base sequence of at least the sequence of interest is compared to a second nucleotide base sequence of at least the sequence of interest obtained from a NGS method of sequencing performed on a second aliquot of the plurality of amplicons.

49. The method of claim 48, wherein the NGS method of sequencing is semiconductor sequencing.

50. The method of any one of claims 35-49, wherein the second reaction mixture further comprises a polymerase, deoxynucleotide triphosphates, and dye-labelled dideoxynucleotide triphosphates.

51. The method of any one of claims 35-50, wherein amplifying DNA comprises polymerase chain reaction amplification.

52. The method of any one of claims 35-50, wherein the first reaction mixture further comprises a polymerase.

53. The method of claim 52, wherein the polymerase is a thermostable polymerase.

54. The method of any one of claims 52-53, wherein the polymerase is Taq polymerase.

55. The method of any one of claims 35-55, wherein the polymerase of the second reaction mixture is a thermostable polymerase.

56. The method of claim 55, wherein the polymerase is Taq polymerase.

57. The method of any one of claims 35-56, wherein the nuclease is selected from exonuclease I, Exo III, Pfu and DNA pol I.

58. The method of any one of claims 35-57, wherein the chemically-enhanced primer comprises an oligonucleotide sequence, a NCM and none or at least one nuclease-resistant linkage.

59. The method of any one of claims 35-58, wherein the chemically-enhanced primer comprises one nuclease-resistant linkage at a terminal 3′ end.

60. The method of any one of claims 35-59, wherein the sequencing reaction comprises cycle sequencing.

61. The method of any one of claims 35-60, wherein the chemically-enhanced primer comprises a plurality of NCMs either at a terminal 5′ end or within a oligonucleotide sequence of the chemically-enhanced primer.

62. The method of any one of claims 35-61, wherein the NCM is a (Cn) spacer wherein n can be any integer from 1 to 9.

63. The method of claim 62, wherein the NCM comprises a plurality of (Cn) spacers.

64. The method of any one of claims 35-63, wherein the chemically-enhanced primer has a structure of the formula:
(Cn)ₓ-OLIGO, wherein (Cn)ₓ has a structure of the following formula: 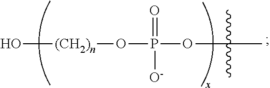
wherein each instance of n is independently an integer of 1 to 9; and x is an integer of 1 to about 30;
OLIGO has a structure of the following formula: 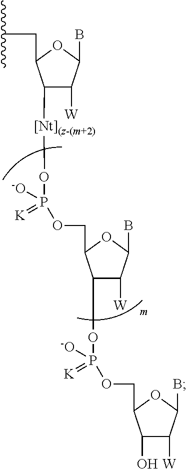
wherein B is a nucleobase;
K is S or O;
m is 0 or 1;
z is an integer of 3 to about 100;
W is OH, F, OMe, or H; and
Nt is a moiety having a formula:

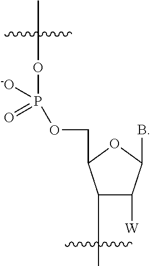

65. The method of any one of claims 35-64, wherein each of the plurality of nuclease-sensitive amplification primers is configured to prime a sequence of interest of a specific disease state.

66. The method of claim 65, wherein the plurality of nuclease-sensitive amplification primers prime a set of sequences connected to a specific disease state.

67. A method of preparing DNA for sequencing, comprising the steps of:
a) amplifying a sample comprising nucleic acid using at least a first priming sequence to provide a plurality of amplicons, wherein each of the plurality of amplicons comprises a sequence of interest and a preceding sequence 5′ to the sequence of interest incorporated from a first priming sequence;
b) amplifying an aliquot of the plurality of amplicons in a first reaction mixture comprising nuclease-sensitive amplification primers to form an amplified DNA product;
c) contacting the first reaction mixture containing the amplified DNA product with a second reaction mixture comprising a nuclease and a chemically-enhanced primer whereby the nuclease sensitive amplification primers are degraded by the nuclease;
d) inactivating the nuclease;
e) priming the amplified DNA product with the chemically-enhanced primer in a sequencing reaction; and
f) producing extension products of the chemically enhanced primer.

68. A method of sequencing and verifying a variant nucleic acid sequence of interest, comprising the steps:
a) amplifying a sample comprising nucleic acid using at least a first priming sequence to provide a plurality of amplicons, wherein each of the plurality of amplicons comprises a sequence of interest and a preceding sequence 5′ to the sequence of interest incorporated from a first priming sequence;
b) splitting the plurality of amplicons into a first aliquot and a second aliquot;
c) amplifying the first aliquot of the plurality of amplicons in a first reaction mixture comprising nuclease-sensitive amplification primers to form a first amplified DNA product;
d) contacting the first reaction mixture containing the first amplified DNA product with a second reaction mixture comprising a nuclease and a chemically-enhanced primer whereby the nuclease sensitive amplification primers are degraded by the nuclease;
e) inactivating the nuclease;
f) priming the first amplified DNA product with the chemically-enhanced primer in a sequencing reaction;
g) producing extension products of the chemically enhanced primer;
h) obtaining sequencing results of at least the sequence of interest of the extended chemically enhanced primer using a mobility dependent separation; and
i) determining a nucleotide base sequence of at least the sequence of interest of the extended chemically enhanced primer;
j) amplifying the second aliquot of the amplicons to form a second DNA product;
k) obtaining sequencing results of at least the sequence of interest of the second DNA product using a NGS sequencing method; and
l) verifying a nucleotide sequence of the second DNA product by comparing it to the nucleotide base sequence of at least the sequence of interest of the extended chemically enhanced primer.

69. The method of claim 68, wherein the step of amplifying the second aliquot of the plurality of amplicons to form a second DNA product further comprises at least one of ligating adaptors, binding to beads, and ligating barcodes.

70. A composition for sequencing nucleic acid comprising:
a PCR amplification reaction product that comprises:
a) a DNA product amplified from at least one amplicon, wherein the amplicon comprises a sequence of interest and a preceding sequence 5′ to the sequence of interest incorporated from a first priming sequence; and
b) non-nuclease-resistant amplification primer(s); and
c) a chemically enhanced primer wherein the chemically enhanced primer comprises an oligonucleotide sequence, a NCM and none or at least one nuclease-resistant linkage.

71. The composition of claim 70, wherein the chemically-enhanced primer comprises a plurality of NCMs either at a terminal 5′ end or within a oligonucleotide sequence of the chemically-enhanced primer.

72. The composition of any one of claims 70-71, wherein the NCM is a (Cn) spacer wherein n can be any integer from 1 to 9.

73. The composition of any one of claims 70-73, wherein the NCM comprises a plurality of (Cn) spacers.

74. The composition of any one of claims 70-73, wherein the chemically-enhanced primer has a structure of Formula I: 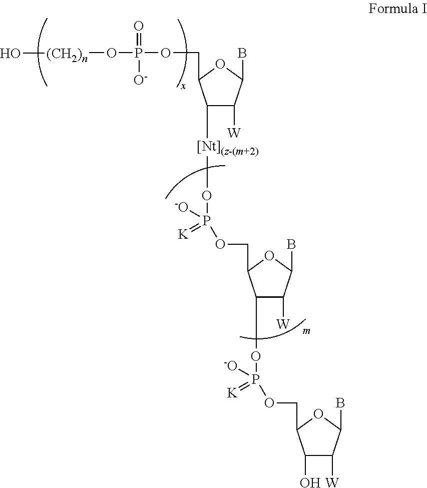
wherein B is a nucleobase;
K is S or O;
each n is independently an integer of 1 to 9;
m is 0 or 1;
x is an integer of 1 to about 30;
z is an integer of 3 to about 100;
W is OH, F, OMe, or H; and
Nt is a moiety having a formula:

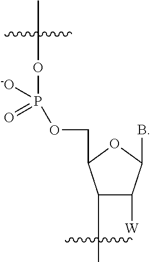

75. The composition of any one of claims 70-74, wherein the oligonucleotide portion of the chemically-enhanced primer comprises a universal primer.

76. The composition of any one of claims 70-75, wherein the universal primer is selected from M13, US1, T7, SP6, and T3.

77. The composition of any one of claims 70-76, wherein the chemically-enhanced primer comprises one nuclease-resistant linkage.

78. The composition of any one of claims 70-77, further comprising a polymerase, a nuclease, a deoxynucleotide triphosphates, dideoxynucleotide triphosphates and a dye-label.

79. The composition of any one of claims 70-78, wherein the dideoxynucleotide triphosphates comprise dideoxynucleotide triphosphates labeled with the dye-label.

80. The composition of any one of claims 70-79, wherein the dye-label is attached to the NCM or the oligonucleotide sequence.

81. The composition of any one of claims 70-80, wherein the nuclease is selected from exonuclease I, Exo Ill, Pfu and DNA pol I.

82. The composition of any one of claims 70-81, wherein the dye-labeled dideoxynucleotide triphosphates comprise fluorescent dye-labeled dideoxynucleotide triphosphates.

83. The composition of any one of claims 70-82, wherein the PCR amplification reaction product further comprises an amplified DNA product wherein the DNA product is the amplification product of a plurality of amplicons.

84. The composition of any one of claims 70-83, wherein the polymerase is Taq polymerase.

85. The composition of any one of claims 70-84, wherein the universal primer is M13.

86. A chemically enhanced primer, comprising: an oligonucleotide sequence, at least one NCM and none or at least one nuclease-resistant linkage, and wherein at least the 10 of the nucleotides at a 3′ terminus of the chemically enhanced primer are complementary to at least 10 of the nucleotides at the 5′ terminus of an amplicon, wherein the amplicon comprises a sequence of interest and a preceding sequence 5′ to the sequence of interest incorporated from a first priming sequence.

87. The chemically enhanced primer of claim 86, wherein the chemically-enhanced primer comprises one nuclease-resistant linkage at the terminal 3′ end.

88. The chemically enhanced primer of any one of claims 86-87, wherein the chemically-enhanced primer comprises a plurality of NCMs either at a terminal 5′ end or within an oligonucleotide sequence of the chemically-enhanced primer.

89. The chemically enhanced primer of any one of claims 86-88, wherein the NCM is a (Cn) spacer wherein n is any integer from 1 to 9.

90. The chemically enhanced primer of any one of claims 86-90, wherein the NCM comprises a plurality of (Cn) spacers.

91. The chemically enhanced primer of any one of claims 86-91, wherein the chemically-enhanced primer has a structure of the formula:
(Cn)ₓ-OLIGO, wherein (Cn)ₓ has a structure of the following formula: 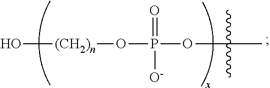
wherein each instance of n is independently an integer of 1 to 9; and x is an integer of 1 to about 30;
OLIGO has a structure of the following formula: 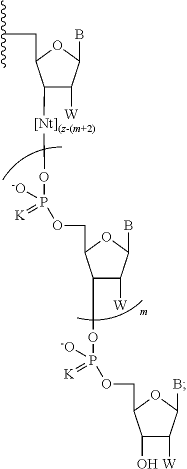
wherein B is a nucleobase;
K is S or O;
m is 0 or 1;
z is an integer of 3 to about 100;
W is OH, F, OMe, or H; and
Nt is a moiety having a formula:

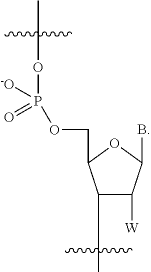

92. A kit, comprising: a polymerase, a nuclease, at least one deoxynucleotide triphosphate, and dideoxynucleotide triphosphates.

93. The kit of claim 92, wherein the dideoxynucleotide triphosphates comprise dideoxynucleotide triphosphates labeled with a dye-label.

94. The kit of any one of claims 92-93, wherein the dye-labeled dideoxynucleotide triphosphates comprise fluorescent dye-labeled dideoxynucleotide triphosphates.

95. The kit of any one of claims 92-94, wherein the nuclease is selected from exonuclease I, Exo Ill, Pfu and DNA pol I.

96. The kit of any one of claims 92-95, further comprising the chemically enhanced primer of any one of claims 86-91.

97. The kit of any one of claims 92-96, further comprising a plurality of nuclease sensitive amplification primers.

98. The kit of any one of claims 92-97, wherein each of the plurality of nuclease- sensitive amplification primers is configured to prime a sequence of interest of a specific disease state.

99. The kit of any one of claims 92-98, wherein the plurality of nuclease-sensitive amplification primers prime a set of sequences connected to a specific disease state.


SEQUENCE LISTING (371 sequences)

SEQ ID NO: 1 - DNA, 18 bp, Artificial Sequence
  source: /note=“Description of Artificial Sequence Synthetic primer”
tgtaaaacgacggccagt

SEQ ID NO: 2 - DNA, 18 bp, Artificial Sequence
  source: /note=“Description of Artificial Sequence Synthetic primer”
caggaaacagctatgacc

SEQ ID NO: 3 - DNA, 20 bp, Artificial Sequence
  source: /note=“Description of Artificial Sequence Synthetic primer”
taatacgactcactataggg

SEQ ID NO: 4 - DNA, 18 bp, Artificial Sequence
  source: /note=“Description of Artificial Sequence Synthetic primer”
atttaggtgacactatag

SEQ ID NO: 5 - DNA, 20 bp, Artificial Sequence
  source: /note=“Description of Artificial Sequence Synthetic primer”
attaaccctcactaaaggga

SEQ ID NO: 6 - DNA, 21 bp, Artificial Sequence
  source: /note=“Description of Artificial Sequence Synthetic primer”
tctatggtgtgtcccccaact

SEQ ID NO: 7 - DNA, 23 bp, Artificial Sequence
  source: /note=“Description of Artificial Sequence Synthetic primer”
cgtcaggctgtatttcttccaca

SEQ ID NO: 8 - DNA, 21 bp, Artificial Sequence
  source: /note=“Description of Artificial Sequence Synthetic primer”
cgctgaagctccattttgcat

SEQ ID NO: 9 - DNA, 25 bp, Artificial Sequence
  source: /note=“Description of Artificial Sequence Synthetic primer”
cagcttctttcaagaactcttccac

SEQ ID NO: 10 - DNA, 23 bp, Artificial Sequence
  source: /note=“Description of Artificial Sequence Synthetic primer”
ggagaaccacttggtgaaggtag

SEQ ID NO: 11 - DNA, 25 bp, Artificial Sequence
  source: /note=“Description of Artificial Sequence Synthetic primer”
cggacttgatggagaacttgttgta

SEQ ID NO: 12 - DNA, 21 bp, Artificial Sequence
  source: /note=“Description of Artificial Sequence Synthetic primer”
gcgccacagagaagttgttga

SEQ ID NO: 13 - DNA, 20 bp, Artificial Sequence
  source: /note=“Description of Artificial Sequence Synthetic primer”
gggtctgacgggtagagtgt

SEQ ID NO: 14 - DNA, 21 bp, Artificial Sequence
  source: /note=“Description of Artificial Sequence Synthetic primer”
cttggccacgatgacttcctt

SEQ ID NO: 15 - DNA, 24 bp, Artificial Sequence
  source: /note=“Description of Artificial Sequence Synthetic primer”
ccatgaacgagtttgagtacctga

SEQ ID NO: 16 - DNA, 22 bp, Artificial Sequence
  source: /note=“Description of Artificial Sequence Synthetic primer”
tctctcggaggaaggacttgag

SEQ ID NO: 17 - DNA, 22 bp, Artificial Sequence
  source: /note=“Description of Artificial Sequence Synthetic primer”
gcccagactcagctcagttaat

SEQ ID NO: 18 - DNA, 25 bp, Artificial Sequence
  source: /note=“Description of Artificial Sequence Synthetic primer”
acagggtaccaggagatgatgtaag

SEQ ID NO: 19 - DNA, 21 bp, Artificial Sequence
  source: /note=“Description of Artificial Sequence Synthetic primer”
ggaagagtggccaagattgga

SEQ ID NO: 20 - DNA, 22 bp, Artificial Sequence
  source: /note=“Description of Artificial Sequence Synthetic primer”
gagagaacgcggaattggtcta

SEQ ID NO: 21 - DNA, 25 bp, Artificial Sequence
  source: /note=“Description of Artificial Sequence Synthetic primer”
gtatgaatggctgacacttcttcca

SEQ ID NO: 22 - DNA, 26 bp, Artificial Sequence
  source: /note=“Description of Artificial Sequence Synthetic primer”
agcactgatgataaacacctcaagtt

SEQ ID NO: 23 - DNA, 24 bp, Artificial Sequence
  source: /note=“Description of Artificial Sequence Synthetic primer”
atcttcttgacacaaagactggct

SEQ ID NO: 24 - DNA, 28 bp, Artificial Sequence
  source: /note=“Description of Artificial Sequence Synthetic primer”
ttcattatcatctttgtcatcagctgaa

SEQ ID NO: 25 - DNA, 22 bp, Artificial Sequence
  source: /note=“Description of Artificial Sequence Synthetic primer”
tttggttctagggtgctgtgac

SEQ ID NO: 26 - DNA, 21 bp, Artificial Sequence
  source: /note=“Description of Artificial Sequence Synthetic primer”
gcagactgcagggttctagtt

SEQ ID NO: 27 - DNA, 25 bp, Artificial Sequence
  source: /note=“Description of Artificial Sequence Synthetic primer”
gtgaactgacagaagtacatctgct

SEQ ID NO: 28 - DNA, 22 bp, Artificial Sequence
  source: /note=“Description of Artificial Sequence Synthetic primer”
agccccagtgatcttccagata

SEQ ID NO: 29 - DNA, 23 bp, Artificial Sequence
  source: /note=“Description of Artificial Sequence Synthetic primer”
ccctctgaactgcagcatttact

SEQ ID NO: 30 - DNA, 20 bp, Artificial Sequence
  source: /note=“Description of Artificial Sequence Synthetic primer”
agagggtccaggttcttcca

SEQ ID NO: 31 - DNA, 24 bp, Artificial Sequence
  source: /note=“Description of Artificial Sequence Synthetic primer”
tcattttcctgaactggaggcatt

SEQ ID NO: 32 - DNA, 26 bp, Artificial Sequence
  source: /note=“Description of Artificial Sequence Synthetic primer”
atgaaacagaatcagagcagcctaaa

SEQ ID NO: 33 - DNA, 22 bp, Artificial Sequence
  source: /note=“Description of Artificial Sequence Synthetic primer”
cgtgatgactttgttggcatgg

SEQ ID NO: 34 - DNA, 30 bp, Artificial Sequence
  source: /note=“Description of Artificial Sequence Synthetic primer”
atgatctgctagtgaatgagataagtcata

SEQ ID NO: 35 - DNA, 29 bp, Artificial Sequence
  source: /note=“Description of Artificial Sequence Synthetic primer”
acaggaaatttctaaatgtgacatgacct

SEQ ID NO: 36 - DNA, 28 bp, Artificial Sequence
  source: /note=“Description of Artificial Sequence Synthetic primer”
agttctgttaaagttcatggcttttgtg

SEQ ID NO: 37 - DNA, 24 bp, Artificial Sequence
  source: /note=“Description of Artificial Sequence Synthetic primer”
agcgtttacgatcctctttcagtg

SEQ ID NO: 38 - DNA, 27 bp, Artificial Sequence
  source: /note=“Description of Artificial Sequence Synthetic primer”
acaggagaatatggaaatctggtgact

SEQ ID NO: 39 - DNA, 24 bp, Artificial Sequence
  source: /note=“Description of Artificial Sequence Synthetic primer”
tcaccttaacaagctgtctcctct

SEQ ID NO: 40 - DNA, 28 bp, Artificial Sequence
  source: /note=“Description of Artificial Sequence Synthetic primer”
tgctattctcagatgactctgtgttttt

SEQ ID NO: 41 - DNA, 24 bp, Artificial Sequence
  source: /note=“Description of Artificial Sequence Synthetic primer”
ttccgtgtttctctgcagtaatgt

SEQ ID NO: 42 - DNA, 31 bp, Artificial Sequence
  source: /note=“Description of Artificial Sequence Synthetic primer”
gtttgtttcttttttctccagttggttacat

SEQ ID NO: 43 - DNA, 24 bp, Artificial Sequence
  source: /note=“Description of Artificial Sequence Synthetic primer”
tcttggtaggcaaacaacattcca

SEQ ID NO: 44 - DNA, 26 bp, Artificial Sequence
  source: /note=“Description of Artificial Sequence Synthetic primer”
gtgtttgactctagatgctgtgagaa

SEQ ID NO: 45 - DNA, 29 bp, Artificial Sequence
  source: /note=“Description of Artificial Sequence Synthetic primer”
tgagatacacagtctacctggtaagaaaa

SEQ ID NO: 46 - DNA, 25 bp, Artificial Sequence
  source: /note=“Description of Artificial Sequence Synthetic primer”
cacctcactgaaacctttgtgtttt

SEQ ID NO: 47 - DNA, 24 bp, Artificial Sequence
  source: /note=“Description of Artificial Sequence Synthetic primer”
aattcacttgtccaccaacactga

SEQ ID NO: 48 - DNA, 21 bp, Artificial Sequence
  source: /note=“Description of Artificial Sequence Synthetic primer”
gctcatacagcaggccataga

SEQ ID NO: 49 - DNA, 28 bp, Artificial Sequence
  source: /note=“Description of Artificial Sequence Synthetic primer”
gtatgttggcaggttaaaaataaaggct

SEQ ID NO: 50 - DNA, 28 bp, Artificial Sequence
  source: /note=“Description of Artificial Sequence Synthetic primer”
tcaccttcagaagtcacagaatgatttt

SEQ ID NO: 51 - DNA, 28 bp, Artificial Sequence
  source: /note=“Description of Artificial Sequence Synthetic primer”
ttgagatgaaaggattccactgaaagtt

SEQ ID NO: 52 - DNA, 29 bp, Artificial Sequence
  source: /note=“Description of Artificial Sequence Synthetic primer”
agtattctttacatggcttttggtcttct

SEQ ID NO: 53 - DNA, 21 bp, Artificial Sequence
  source: /note=“Description of Artificial Sequence Synthetic primer”
cattctggcacgctttggaaa

SEQ ID NO: 54 - DNA, 22 bp, Artificial Sequence
  source: /note=“Description of Artificial Sequence Synthetic primer”
aaaaagccatttgaccgtggag

SEQ ID NO: 55 - DNA, 23 bp, Artificial Sequence
  source: /note=“Description of Artificial Sequence Synthetic primer”
caggttcgtttgcatcactaaca

SEQ ID NO: 56 - DNA, 27 bp, Artificial Sequence
  source: /note=“Description of Artificial Sequence Synthetic primer”
acagactgctttccaaagattcttgta

SEQ ID NO: 57 - DNA, 27 bp, Artificial Sequence
  source: /note=“Description of Artificial Sequence Synthetic primer”
gccatacctgttttcccaataagtttt

SEQ ID NO: 58 - DNA, 26 bp, Artificial Sequence
  source: /note=“Description of Artificial Sequence Synthetic primer”
ctatatgtagaggctgttggaagctg

SEQ ID NO: 59 - DNA, 25 bp, Artificial Sequence
  source: /note=“Description of Artificial Sequence Synthetic primer”
tcagcattatgaaggtccactgaag

SEQ ID NO: 60 - DNA, 27 bp, Artificial Sequence
  source: /note=“Description of Artificial Sequence Synthetic primer”
actggtgtacttgataggcatttgaat

SEQ ID NO: 61 - DNA, 24 bp, Artificial Sequence
  source: /note=“Description of Artificial Sequence Synthetic primer”
tgtagataggccagcattggatct

SEQ ID NO: 62 - DNA, 22 bp, Artificial Sequence
  source: /note=“Description of Artificial Sequence Synthetic primer”
aaacccttttgaaggcctggat

SEQ ID NO: 63 - DNA, 22 bp, Artificial Sequence
  source: /note=“Description of Artificial Sequence Synthetic primer”
tcacattttgtgcctccactgt

SEQ ID NO: 64 - DNA, 30 bp, Artificial Sequence
  source: /note=“Description of Artificial Sequence Synthetic primer”
tcatttttctttagaccttcttcaggaaca

SEQ ID NO: 65 - DNA, 23 bp, Artificial Sequence
  source: /note=“Description of Artificial Sequence Synthetic primer”
tgtaaagtgagcagcacaagact

SEQ ID NO: 66 - DNA, 25 bp, Artificial Sequence
  source: /note=“Description of Artificial Sequence Synthetic primer”
catacttaccatgccactttccctt

SEQ ID NO: 67 - DNA, 27 bp, Artificial Sequence
  source: /note=“Description of Artificial Sequence Synthetic primer”
tttctttttctgtttggcttgacttga

SEQ ID NO: 68 - DNA, 25 bp, Artificial Sequence
  source: /note=“Description of Artificial Sequence Synthetic primer”
ccacaaaatggatccagacaactgt

SEQ ID NO: 69 - DNA, 28 bp, Artificial Sequence
  source: /note=“Description of Artificial Sequence Synthetic primer”
gcttgctctgataggaaaatgagatcta

SEQ ID NO: 70 - DNA, 20 bp, Artificial Sequence
  source: /note=“Description of Artificial Sequence Synthetic primer”
agattgcaccggtcgacaaa

SEQ ID NO: 71 - DNA, 22 bp, Artificial Sequence
  source: /note=“Description of Artificial Sequence Synthetic primer”
cccaggcgtagaccaagaaatg

SEQ ID NO: 72 - DNA, 21 bp, Artificial Sequence
  source: /note=“Description of Artificial Sequence Synthetic primer”
tggtcctgacttggttgtgtc

SEQ ID NO: 73 - DNA, 28 bp, Artificial Sequence
  source: /note=“Description of Artificial Sequence Synthetic primer”
gttatagaattaccgtggtgggattgaa

SEQ ID NO: 74 - DNA, 28 bp, Artificial Sequence
  source: /note=“Description of Artificial Sequence Synthetic primer”
gcagtcttggtactttgtaaatgacaca

SEQ ID NO: 75 - DNA, 25 bp, Artificial Sequence
  source: /note=“Description of Artificial Sequence Synthetic primer”
tcaatatggtgtatacagcctccca

SEQ ID NO: 76 - DNA, 23 bp, Artificial Sequence
  source: /note=“Description of Artificial Sequence Synthetic primer”
tccaccatgactttgaggttgag

SEQ ID NO: 77 - DNA, 22 bp, Artificial Sequence
  source: /note=“Description of Artificial Sequence Synthetic primer”
tcccactaatgccagatgcttg

SEQ ID NO: 78 - DNA, 23 bp, Artificial Sequence
  source: /note=“Description of Artificial Sequence Synthetic primer”
ccatccatggaggagttgaagtt

SEQ ID NO: 79 - DNA, 21 bp, Artificial Sequence
  source: /note=“Description of Artificial Sequence Synthetic primer”
ctctagtgagcacctgacctg

SEQ ID NO: 80 - DNA, 31 bp, Artificial Sequence
  source: /note=“Description of Artificial Sequence Synthetic primer”
actgtttcgtatttatagctgatttgatgga

SEQ ID NO: 81 - DNA, 22 bp, Artificial Sequence
  source: /note=“Description of Artificial Sequence Synthetic primer”
cctcttcctcaggattgccttt

SEQ ID NO: 82 - DNA, 21 bp, Artificial Sequence
  source: /note=“Description of Artificial Sequence Synthetic primer”
cctcattgccctcaacacagt

SEQ ID NO: 83 - DNA, 28 bp, Artificial Sequence
  source: /note=“Description of Artificial Sequence Synthetic primer”
tcagtccggttttatttgcatcatagtt

SEQ ID NO: 84 - DNA, 21 bp, Artificial Sequence
  source: /note=“Description of Artificial Sequence Synthetic primer”
caccacgtaccagatggatgt

SEQ ID NO: 85 - DNA, 25 bp, Artificial Sequence
  source: /note=“Description of Artificial Sequence Synthetic primer”
cccaaagactctccaagatgggata

SEQ ID NO: 86 - DNA, 26 bp, Artificial Sequence
  source: /note=“Description of Artificial Sequence Synthetic primer”
agacatgcatgaacatttttctccac

SEQ ID NO: 87 - DNA, 22 bp, Artificial Sequence
  source: /note=“Description of Artificial Sequence Synthetic primer”
tccagaccagggtgttgttttc

SEQ ID NO: 88 - DNA, 20 bp, Artificial Sequence
  source: /note=“Description of Artificial Sequence Synthetic primer”
tgtggagcctcttacaccca

SEQ ID NO: 89 - DNA, 23 bp, Artificial Sequence
  source: /note=“Description of Artificial Sequence Synthetic primer”
gtgccagggaccttaccttatac

SEQ ID NO: 90 - DNA, 23 bp, Artificial Sequence
  source: /note=“Description of Artificial Sequence Synthetic primer”
acgtcttccttctctctctgtca

SEQ ID NO: 91 - DNA, 20 bp, Artificial Sequence
  source: /note=“Description of Artificial Sequence Synthetic primer”
ctgaggttcagagccatgga

SEQ ID NO: 92 - DNA, 19 bp, Artificial Sequence
  source: /note=“Description of Artificial Sequence Synthetic primer”
catgcgaagccacactgac

SEQ ID NO: 93 - DNA, 20 bp, Artificial Sequence
  source: /note=“Description of Artificial Sequence Synthetic primer”
cggacatagtccaggaggca

SEQ ID NO: 94 - DNA, 23 bp, Artificial Sequence
  source: /note=“Description of Artificial Sequence Synthetic primer”
gactatgtccgggaacacaaaga

SEQ ID NO: 95 - DNA, 21 bp, Artificial Sequence
  source: /note=“Description of Artificial Sequence Synthetic primer”
ccccatggcaaactcttgcta

SEQ ID NO: 96 - DNA, 23 bp, Artificial Sequence
  source: /note=“Description of Artificial Sequence Synthetic primer”
cgcagcatgtcaagatcacagat

SEQ ID NO: 97 - DNA, 26 bp, Artificial Sequence
  source: /note=“Description of Artificial Sequence Synthetic primer”
gcatgtgttaaacaatacagctagtg

SEQ ID NO: 98 - DNA, 25 bp, Artificial Sequence
  source: /note=“Description of Artificial Sequence Synthetic primer”
gaatgtgaaaattccagtggccatc

SEQ ID NO: 99 - DNA, 23 bp, Artificial Sequence
  source: /note=“Description of Artificial Sequence Synthetic primer”
gtcatatctccccaaaccccaat

SEQ ID NO: 100 - DNA, 20 bp, Artificial Sequence
  source: /note=“Description of Artificial Sequence Synthetic primer”
gggtgtgtggtctcccatac

SEQ ID NO: 101 - DNA, 22 bp, Artificial Sequence
  source: /note=“Description of Artificial Sequence Synthetic primer”
gccatagggcataagctgtgtc

SEQ ID NO: 102 - DNA, 22 bp, Artificial Sequence
  source: /note=“Description of Artificial Sequence Synthetic primer”
ggatgagctacctggaggatgt

SEQ ID NO: 103 - DNA, 23 bp, Artificial Sequence
  source: /note=“Description of Artificial Sequence Synthetic primer”
ccttggtccttcacctaaccttg

SEQ ID NO: 104 - DNA, 27 bp, Artificial Sequence
  source: /note=“Description of Artificial Sequence Synthetic primer”
gccttagagtgttcctcaatgtaacaa

SEQ ID NO: 105 - DNA, 27 bp, Artificial Sequence
  source: /note=“Description of Artificial Sequence Synthetic primer”
gaaactttggacttcaagaacttggat

SEQ ID NO: 106 - DNA, 25 bp, Artificial Sequence
  source: /note=“Description of Artificial Sequence Synthetic primer”
catcgccacatagggtagaacattt

SEQ ID NO: 107 - DNA, 27 bp, Artificial Sequence
  source: /note=“Description of Artificial Sequence Synthetic primer”
cagacaccattcattggcaagatattg

SEQ ID NO: 108 - DNA, 26 bp, Artificial Sequence
  source: /note=“Description of Artificial Sequence Synthetic primer”
cctgaatcaaatagggaaggaaagga

SEQ ID NO: 109 - DNA, 20 bp, Artificial Sequence
  source: /note=“Description of Artificial Sequence Synthetic primer”
ggcagatgctacggacctta

SEQ ID NO: 110 - DNA, 28 bp, Artificial Sequence
  source: /note=“Description of Artificial Sequence Synthetic primer”
tctgttacttacgtggacatttcttgac

SEQ ID NO: 111 - DNA, 23 bp, Artificial Sequence
  source: /note=“Description of Artificial Sequence Synthetic primer”
caggcctgcatgaatttcaatga

SEQ ID NO: 112 - DNA, 23 bp, Artificial Sequence
  source: /note=“Description of Artificial Sequence Synthetic primer”
ggcaaatgtcagtgcaaggttta

SEQ ID NO: 113 - DNA, 25 bp, Artificial Sequence
  source: /note=“Description of Artificial Sequence Synthetic primer”
tgttttgagcttgtttgctgaatgt

SEQ ID NO: 114 - DNA, 27 bp, Artificial Sequence
  source: /note=“Description of Artificial Sequence Synthetic primer”
acccatgaataccagtgactagaaaga

SEQ ID NO: 115 - DNA, 25 bp, Artificial Sequence
  source: /note=“Description of Artificial Sequence Synthetic primer”
ctcaatcccctaactctgagtcttg

SEQ ID NO: 116 - DNA, 22 bp, Artificial Sequence
  source: /note=“Description of Artificial Sequence Synthetic primer”
gccagcaagaatgcttaccctt

SEQ ID NO: 117 - DNA, 23 bp, Artificial Sequence
  source: /note=“Description of Artificial Sequence Synthetic primer”
gggtcctgacaactgtacaaagt

SEQ ID NO: 118 - DNA, 26 bp, Artificial Sequence
  source: /note=“Description of Artificial Sequence Synthetic primer”
catttgaccatgaccatgtaaacgtc

SEQ ID NO: 119 - DNA, 23 bp, Artificial Sequence
  source: /note=“Description of Artificial Sequence Synthetic primer”
ggaactgatgacctttggaggaa

SEQ ID NO: 120 - DNA, 26 bp, Artificial Sequence
  source: /note=“Description of Artificial Sequence Synthetic primer”
acaatgccacctgaatacaggttatc

SEQ ID NO: 121 - DNA, 22 bp, Artificial Sequence
  source: /note=“Description of Artificial Sequence Synthetic primer”
gcatctattgctggcaccatct

SEQ ID NO: 122 - DNA, 27 bp, Artificial Sequence
  source: /note=“Description of Artificial Sequence Synthetic primer”
tgacaatgtttaaaggtggtagctgtt

SEQ ID NO: 123 - DNA, 27 bp, Artificial Sequence
  source: /note=“Description of Artificial Sequence Synthetic primer”
actcattgatagttgtgaaccaacaca

SEQ ID NO: 124 - DNA, 25 bp, Artificial Sequence
  source: /note=“Description of Artificial Sequence Synthetic primer”
cctgtgactgctgaccaaactttta

SEQ ID NO: 125 - DNA, 27 bp, Artificial Sequence
  source: /note=“Description of Artificial Sequence Synthetic primer”
cacatctttcttataggtgctgaaagg

SEQ ID NO: 126 - DNA, 23 bp, Artificial Sequence
  source: /note=“Description of Artificial Sequence Synthetic primer”
cccaaccatgacaagattttccc

SEQ ID NO: 127 - DNA, 27 bp, Artificial Sequence
  source: /note=“Description of Artificial Sequence Synthetic primer”
ggtcatcacaaatgagagacaacatca

SEQ ID NO: 128 - DNA, 24 bp, Artificial Sequence
  source: /note=“Description of Artificial Sequence Synthetic primer”
actaacaaccctcctgccatcata

SEQ ID NO: 129 - DNA, 21 bp, Artificial Sequence
  source: /note=“Description of Artificial Sequence Synthetic primer”
tctgcagagttgttagcggtt

SEQ ID NO: 130 - DNA, 25 bp, Artificial Sequence
  source: /note=“Description of Artificial Sequence Synthetic primer”
gtagaatctgcattcccagagacaa

SEQ ID NO: 131 - DNA, 27 bp, Artificial Sequence
  source: /note=“Description of Artificial Sequence Synthetic primer”
tctcttgatacatcaatccgtgtttgg

SEQ ID NO: 132 - DNA, 24 bp, Artificial Sequence
  source: /note=“Description of Artificial Sequence Synthetic primer”
acccaaagggcagtaagataggaa

SEQ ID NO: 133 - DNA, 20 bp, Artificial Sequence
  source: /note=“Description of Artificial Sequence Synthetic primer”
ggtccctaggaggaacctca

SEQ ID NO: 134 - DNA, 25 bp, Artificial Sequence
  source: /note=“Description of Artificial Sequence Synthetic primer”
ggtcactgtacaccttacacatgaa

SEQ ID NO: 135 - DNA, 21 bp, Artificial Sequence
  source: /note=“Description of Artificial Sequence Synthetic primer”
ccctctttagccatggcaagg

SEQ ID NO: 136 - DNA, 26 bp, Artificial Sequence
  source: /note=“Description of Artificial Sequence Synthetic primer”
catcactgtaaaccttgcagacaaac

SEQ ID NO: 137 - DNA, 22 bp, Artificial Sequence
  source: /note=“Description of Artificial Sequence Synthetic primer”
tggtctctcattctcccatccc

SEQ ID NO: 138 - DNA, 23 bp, Artificial Sequence
  source: /note=“Description of Artificial Sequence Synthetic primer”
catcctctctcaactccaacagg

SEQ ID NO: 139 - DNA, 22 bp, Artificial Sequence
  source: /note=“Description of Artificial Sequence Synthetic primer”
agtggatcaagcacgtggaaaa

SEQ ID NO: 140 - DNA, 23 bp, Artificial Sequence
  source: /note=“Description of Artificial Sequence Synthetic primer”
gcttcttggtcgtgttcttcatt

SEQ ID NO: 141 - DNA, 22 bp, Artificial Sequence
  source: /note=“Description of Artificial Sequence Synthetic primer”
ctcctcctgtgatctgcaatct

SEQ ID NO: 142 - DNA, 22 bp, Artificial Sequence
  source: /note=“Description of Artificial Sequence Synthetic primer”
tggaagcccagccatttctaaa

SEQ ID NO: 143 - DNA, 28 bp, Artificial Sequence
  source: /note=“Description of Artificial Sequence Synthetic primer”
gatgatgaagatgattgggaaacacaag

SEQ ID NO: 144 - DNA, 20 bp, Artificial Sequence
  source: /note=“Description of Artificial Sequence Synthetic primer”
gtgaccgaggacaacgtgat

SEQ ID NO: 145 - DNA, 19 bp, Artificial Sequence
  source: /note=“Description of Artificial Sequence Synthetic primer”
gcgtcctactggcatgacc

SEQ ID NO: 146 - DNA, 20 bp, Artificial Sequence
  source: /note=“Description of Artificial Sequence Synthetic primer”
ctctgggagatcttcacgct

SEQ ID NO: 147 - DNA, 19 bp, Artificial Sequence
  source: /note=“Description of Artificial Sequence Synthetic primer”
ccactcacaggtcgtgtgt

SEQ ID NO: 148 - DNA, 20 bp, Artificial Sequence
  source: /note=“Description of Artificial Sequence Synthetic primer”
cgcctttcgagcagtactcc

SEQ ID NO: 149 - DNA, 21 bp, Artificial Sequence
  source: /note=“Description of Artificial Sequence Synthetic primer”
gctagggacccctcacattgt

SEQ ID NO: 150 - DNA, 23 bp, Artificial Sequence
  source: /note=“Description of Artificial Sequence Synthetic primer”
cttgggagacttgtctgaacact

SEQ ID NO: 151 - DNA, 27 bp, Artificial Sequence
  source: /note=“Description of Artificial Sequence Synthetic primer”
gtgagcttatttcacacgttcttttct

SEQ ID NO: 152 - DNA, 26 bp, Artificial Sequence
  source: /note=“Description of Artificial Sequence Synthetic primer”
gcacattccattcttaccaaactcta

SEQ ID NO: 153 - DNA, 27 bp, Artificial Sequence
  source: /note=“Description of Artificial Sequence Synthetic primer”
tgactcatcatttcatctctgaagcaa

SEQ ID NO: 154 - DNA, 27 bp, Artificial Sequence
  source: /note=“Description of Artificial Sequence Synthetic primer”
caaacatcctctttgtcatcaagctac

SEQ ID NO: 155 - DNA, 23 bp, Artificial Sequence
  source: /note=“Description of Artificial Sequence Synthetic primer”
gaggcactcatgtcagaactcaa

SEQ ID NO: 156 - DNA, 28 bp, Artificial Sequence
  source: /note=“Description of Artificial Sequence Synthetic primer”
cgacacaacacaaaatagccgtataaaa

SEQ ID NO: 157 - DNA, 22 bp, Artificial Sequence
  source: /note=“Description of Artificial Sequence Synthetic primer”
ccacgggaaagtggtgaagata

SEQ ID NO: 158 - DNA, 20 bp, Artificial Sequence
  source: /note=“Description of Artificial Sequence Synthetic primer”
ggattgcagattgggccttg

SEQ ID NO: 159 - DNA, 25 bp, Artificial Sequence
  source: /note=“Description of Artificial Sequence Synthetic primer”
acatgatggatgtcacgttctcaaa

SEQ ID NO: 160 - DNA, 28 bp, Artificial Sequence
  source: /note=“Description of Artificial Sequence Synthetic primer”
ataatccattgcctgtctaaagaacact

SEQ ID NO: 161 - DNA, 24 bp, Artificial Sequence
  source: /note=“Description of Artificial Sequence Synthetic primer”
tgttaaccttgcagaatggtcgat

SEQ ID NO: 162 - DNA, 26 bp, Artificial Sequence
  source: /note=“Description of Artificial Sequence Synthetic primer”
ttggtgagatccattgacctcaattt

SEQ ID NO: 163 - DNA, 28 bp, Artificial Sequence
  source: /note=“Description of Artificial Sequence Synthetic primer”
tgaatgtcaagaaaccatgatctctgtt

SEQ ID NO: 164 - DNA, 22 bp, Artificial Sequence
  source: /note=“Description of Artificial Sequence Synthetic primer”
cctctggaataaccagctgtcc

SEQ ID NO: 165 - DNA, 24 bp, Artificial Sequence
  source: /note=“Description of Artificial Sequence Synthetic primer”
tgatccctaacaacacagaagcaa

SEQ ID NO: 166 - DNA, 21 bp, Artificial Sequence
  source: /note=“Description of Artificial Sequence Synthetic primer”
gattgaagagcccacaggtga

SEQ ID NO: 167 - DNA, 21 bp, Artificial Sequence
  source: /note=“Description of Artificial Sequence Synthetic primer”
ctcctccttgctagggttctt

SEQ ID NO: 168 - DNA, 21 bp, Artificial Sequence
  source: /note=“Description of Artificial Sequence Synthetic primer”
tgtccccatcacaggcacagg

SEQ ID NO: 169 - DNA, 20 bp, Artificial Sequence
  source: /note=“Description of Artificial Sequence Synthetic primer”
ggcccgctgtacgtgtccat

SEQ ID NO: 170 - DNA, 21 bp, Artificial Sequence
  source: /note=“Description of Artificial Sequence Synthetic primer”
cgccaggctcacctctatagt

SEQ ID NO: 171 - DNA, 24 bp, Artificial Sequence
  source: /note=“Description of Artificial Sequence Synthetic primer”
ctgaggagcgatgacggaatataa

SEQ ID NO: 172 - DNA, 23 bp, Artificial Sequence
  source: /note=“Description of Artificial Sequence Synthetic primer”
gacttggtgttgttgatggcaaa

SEQ ID NO: 173 - DNA, 20 bp, Artificial Sequence
  source: /note=“Description of Artificial Sequence Synthetic primer”
ctgcaggattcctaccggaa

SEQ ID NO: 174 - DNA, 25 bp, Artificial Sequence
  source: /note=“Description of Artificial Sequence Synthetic primer”
ccaacatgacttacttgatccccat

SEQ ID NO: 175 - DNA, 22 bp, Artificial Sequence
  source: /note=“Description of Artificial Sequence Synthetic primer”
atcaccaaatggcaccatacga

SEQ ID NO: 176 - DNA, 24 bp, Artificial Sequence
  source: /note=“Description of Artificial Sequence Synthetic primer”
tgaagcagcaagtatgatgagcaa

SEQ ID NO: 177 - DNA, 22 bp, Artificial Sequence
  source: /note=“Description of Artificial Sequence Synthetic primer”
ctgacacctagctgtgatcctg

SEQ ID NO: 178 - DNA, 20 bp, Artificial Sequence
  source: /note=“Description of Artificial Sequence Synthetic primer”
ctgattgcatgccagtcctc

SEQ ID NO: 179 - DNA, 22 bp, Artificial Sequence
  source: /note=“Description of Artificial Sequence Synthetic primer”
gcaaggatttggccagtgctat

SEQ ID NO: 180 - DNA, 18 bp, Artificial Sequence
  source: /note=“Description of Artificial Sequence Synthetic primer”
cacgagatgccggtacga

SEQ ID NO: 181 - DNA, 24 bp, Artificial Sequence
  source: /note=“Description of Artificial Sequence Synthetic primer”
gtctgtgagcacaaaatttgggat

SEQ ID NO: 182 - DNA, 21 bp, Artificial Sequence
  source: /note=“Description of Artificial Sequence Synthetic primer”
gatgtcagtctgcccttctgt

SEQ ID NO: 183 - DNA, 23 bp, Artificial Sequence
  source: /note=“Description of Artificial Sequence Synthetic primer”
acttagcttggaagctgacaagt

SEQ ID NO: 184 - DNA, 24 bp, Artificial Sequence
  source: /note=“Description of Artificial Sequence Synthetic primer”
cccctatctctcaagcaaacttca

SEQ ID NO: 185 - DNA, 28 bp, Artificial Sequence
  source: /note=“Description of Artificial Sequence Synthetic primer”
aactatctgttggagaaaagcttgtctt

SEQ ID NO: 186 - DNA, 28 bp, Artificial Sequence
  source: /note=“Description of Artificial Sequence Synthetic primer”
actccgggttacaccatctatagttaag

SEQ ID NO: 187 - DNA, 26 bp, Artificial Sequence
  source: /note=“Description of Artificial Sequence Synthetic primer”
tgctttggaagttcagtcaactcttt

SEQ ID NO: 188 - DNA, 25 bp, Artificial Sequence
  source: /note=“Description of Artificial Sequence Synthetic primer”
cacttctccattcttcacaagggta

SEQ ID NO: 189 - DNA, 25 bp, Artificial Sequence
  source: /note=“Description of Artificial Sequence Synthetic primer”
ggctgcgttggaagttatttctaag

SEQ ID NO: 190 - DNA, 23 bp, Artificial Sequence
  source: /note=“Description of Artificial Sequence Synthetic primer”
aggttgaccacattgagatggtg

SEQ ID NO: 191 - DNA, 26 bp, Artificial Sequence
  source: /note=“Description of Artificial Sequence Synthetic primer”
agggaccccaattattgaaggaaatg

SEQ ID NO: 192 - DNA, 22 bp, Artificial Sequence
  source: /note=“Description of Artificial Sequence Synthetic primer”
gcactagccagtaccttcctct

SEQ ID NO: 193 - DNA, 23 bp, Artificial Sequence
  source: /note=“Description of Artificial Sequence Synthetic primer”
gagcaatccctgtggatctgaaa

SEQ ID NO: 194 - DNA, 25 bp, Artificial Sequence
  source: /note=“Description of Artificial Sequence Synthetic primer”
aagagatttcccaaatgttccacca

SEQ ID NO: 195 - DNA, 24 bp, Artificial Sequence
  source: /note=“Description of Artificial Sequence Synthetic primer”
agcattcaggaagaaagaggcatt

SEQ ID NO: 196 - DNA, 26 bp, Artificial Sequence
  source: /note=“Description of Artificial Sequence Synthetic primer”
gggatgttaggccatatacagtacct

SEQ ID NO: 197 - DNA, 22 bp, Artificial Sequence
  source: /note=“Description of Artificial Sequence Synthetic primer”
gcatggaagaggattctggact

SEQ ID NO: 198 - DNA, 20 bp, Artificial Sequence
  source: /note=“Description of Artificial Sequence Synthetic primer”
ggtgtctgtgtcatcggagt

SEQ ID NO: 199 - DNA, 27 bp, Artificial Sequence
  source: /note=“Description of Artificial Sequence Synthetic primer”
ggtgagggtaaaaagcaaaagaattgt

SEQ ID NO: 200 - DNA, 24 bp, Artificial Sequence
  source: /note=“Description of Artificial Sequence Synthetic primer”
ctctcatgtgatgtccaggagttg

SEQ ID NO: 201 - DNA, 21 bp, Artificial Sequence
  source: /note=“Description of Artificial Sequence Synthetic primer”
ccgtgtactccagtgaggaag

SEQ ID NO: 202 - DNA, 25 bp, Artificial Sequence
  source: /note=“Description of Artificial Sequence Synthetic primer”
cgccaaggaagaagatcatactcaa

SEQ ID NO: 203 - DNA, 21 bp, Artificial Sequence
  source: /note=“Description of Artificial Sequence Synthetic primer”
tttgacaaagcccggatcagt

SEQ ID NO: 204 - DNA, 21 bp, Artificial Sequence
  source: /note=“Description of Artificial Sequence Synthetic primer”
ccacaccctgttcactccttt

SEQ ID NO: 205 - DNA, 24 bp, Artificial Sequence
  source: /note=“Description of Artificial Sequence Synthetic primer”
gtctcagtcattagagcactctgg

SEQ ID NO: 206 - DNA, 26 bp, Artificial Sequence
  source: /note=“Description of Artificial Sequence Synthetic primer”
aaggtgatctatttttccctttctcc

SEQ ID NO: 207 - DNA, 25 bp, Artificial Sequence
  source: /note=“Description of Artificial Sequence Synthetic primer”
tttcatactgaccaaaactcagcct

SEQ ID NO: 208 - DNA, 27 bp, Artificial Sequence
  source: /note=“Description of Artificial Sequence Synthetic primer”
gctttttgctaaaatgcatgtttccaa

SEQ ID NO: 209 - DNA, 21 bp, Artificial Sequence
  source: /note=“Description of Artificial Sequence Synthetic primer”
gacacggctttacctccaatg

SEQ ID NO: 210 - DNA, 30 bp, Artificial Sequence
  source: /note=“Description of Artificial Sequence Synthetic primer”
accttctttctaaccttttcttatgtgctt

SEQ ID NO: 211 - DNA, 30 bp, Artificial Sequence
  source: /note=“Description of Artificial Sequence Synthetic primer”
ctgctttgaacaaataaatgaatcacgttt

SEQ ID NO: 212 - DNA, 21 bp, Artificial Sequence
  source: /note=“Description of Artificial Sequence Synthetic primer”
aggaggtagagcatgacccat

SEQ ID NO: 213 - DNA, 26 bp, Artificial Sequence
  source: /note=“Description of Artificial Sequence Synthetic primer”
gggacaacataagaaactccaggttt

SEQ ID NO: 214 - DNA, 26 bp, Artificial Sequence
  source: /note=“Description of Artificial Sequence Synthetic primer”
cagccagaaatatcctccttactcat

SEQ ID NO: 215 - DNA, 24 bp, Artificial Sequence
  source: /note=“Description of Artificial Sequence Synthetic primer”
gtcaagcagagaatgggtactcac

SEQ ID NO: 216 - DNA, 25 bp, Artificial Sequence
  source: /note=“Description of Artificial Sequence Synthetic primer”
gtgcttctattacaggctcgactac

SEQ ID NO: 217 - DNA, 25 bp, Artificial Sequence
  source: /note=“Description of Artificial Sequence Synthetic primer”
cctaaagagaacagctcccaaagaa

SEQ ID NO: 218 - DNA, 27 bp, Artificial Sequence
  source: /note=“Description of Artificial Sequence Synthetic primer”
caaagaatggtcctgcaccagtaatat

SEQ ID NO: 219 - DNA, 27 bp, Artificial Sequence
  source: /note=“Description of Artificial Sequence Synthetic primer”
aggcctgctgaaaatgactgaatataa

SEQ ID NO: 220 - DNA, 23 bp, Artificial Sequence
  source: /note=“Description of Artificial Sequence Synthetic primer”
tcctcatgtactggtccctcatt

SEQ ID NO: 221 - DNA, 29 bp, Artificial Sequence
  source: /note=“Description of Artificial Sequence Synthetic primer”
gtaaaaggtgcactgtaataatccagact

SEQ ID NO: 222 - DNA, 32 bp, Artificial Sequence
  source: /note=“Description of Artificial Sequence Synthetic primer”
cagatctgtatttatttcagtgttacttacct

SEQ ID NO: 223 - DNA, 27 bp, Artificial Sequence
  source: /note=“Description of Artificial Sequence Synthetic primer”
gactctgaagatgtacctatggtccta

SEQ ID NO: 224 - DNA, 23 bp, Artificial Sequence
  source: /note=“Description of Artificial Sequence Synthetic primer”
ctgacatacagtcggaggttcac

SEQ ID NO: 225 - DNA, 23 bp, Artificial Sequence
  source: /note=“Description of Artificial Sequence Synthetic primer”
agaagttgatgaaccggtccttt

SEQ ID NO: 226 - DNA, 24 bp, Artificial Sequence
  source: /note=“Description of Artificial Sequence Synthetic primer”
caaataggagccagcctgaatgat

SEQ ID NO: 227 - DNA, 27 bp, Artificial Sequence
  source: /note=“Description of Artificial Sequence Synthetic primer”
ggagacatctcacattgtttttgttga

SEQ ID NO: 228 - DNA, 27 bp, Artificial Sequence
  source: /note=“Description of Artificial Sequence Synthetic primer”
catgtcaacatcgctctaattcagaga

SEQ ID NO: 229 - DNA, 27 bp, Artificial Sequence
  source: /note=“Description of Artificial Sequence Synthetic primer”
gcttttcaaaaggcttaaacacaggat

SEQ ID NO: 230 - DNA, 24 bp, Artificial Sequence
  source: /note=“Description of Artificial Sequence Synthetic primer”
cccatgatagccgtctttaacaag

SEQ ID NO: 231 - DNA, 29 bp, Artificial Sequence
  source: /note=“Description of Artificial Sequence Synthetic primer”
cggtagtctacagattcatttgaaaccat

SEQ ID NO: 232 - DNA, 22 bp, Artificial Sequence
  source: /note=“Description of Artificial Sequence Synthetic primer”
gctgattttggtcttgccagag

SEQ ID NO: 233 - DNA, 27 bp, Artificial Sequence
  source: /note=“Description of Artificial Sequence Synthetic primer”
tctgacttggtggtaaacttttgagtt

SEQ ID NO: 234 - DNA, 24 bp, Artificial Sequence
  source: /note=“Description of Artificial Sequence Synthetic primer”
tctgacctcgtcttctacttctgg

SEQ ID NO: 235 - DNA, 26 bp, Artificial Sequence
  source: /note=“Description of Artificial Sequence Synthetic primer”
ccctgccactagaaatatctgtctta

SEQ ID NO: 236 - DNA, 20 bp, Artificial Sequence
  source: /note=“Description of Artificial Sequence Synthetic primer”
tgaccgctctgcatctagtg

SEQ ID NO: 237 - DNA, 23 bp, Artificial Sequence
  source: /note=“Description of Artificial Sequence Synthetic primer”
agcgaaccaagaatgcctgttta

SEQ ID NO: 238 - DNA, 20 bp, Artificial Sequence
  source: /note=“Description of Artificial Sequence Synthetic primer”
cacgcttgaagaccacgttg

SEQ ID NO: 239 - DNA, 19 bp, Artificial Sequence
  source: /note=“Description of Artificial Sequence Synthetic primer”
ggactgtgcggagcatgta

SEQ ID NO: 240 - DNA, 19 bp, Artificial Sequence
  source: /note=“Description of Artificial Sequence Synthetic primer”
acacactgccggttgtcaa

SEQ ID NO: 241 - DNA, 21 bp, Artificial Sequence
  source: /note=“Description of Artificial Sequence Synthetic primer”
cctcaccatgtcctgactgtg

SEQ ID NO: 242 - DNA, 26 bp, Artificial Sequence
  source: /note=“Description of Artificial Sequence Synthetic primer”
gatgtctatgaagtgttgtggttcct

SEQ ID NO: 243 - DNA, 27 bp, Artificial Sequence
  source: /note=“Description of Artificial Sequence Synthetic primer”
gacagccagatatcaactgttacagaa

SEQ ID NO: 244 - DNA, 25 bp, Artificial Sequence
  source: /note=“Description of Artificial Sequence Synthetic primer”
cctcacctctatggtgggatcatat

SEQ ID NO: 245 - DNA, 24 bp, Artificial Sequence
  source: /note=“Description of Artificial Sequence Synthetic primer”
gttcttgctggtgtgaaatgactg

SEQ ID NO: 246 - DNA, 22 bp, Artificial Sequence
  source: /note=“Description of Artificial Sequence Synthetic primer”
ttcgcctgtcctcatgtattgg

SEQ ID NO: 247 - DNA, 24 bp, Artificial Sequence
  source: /note=“Description of Artificial Sequence Synthetic primer”
cacccccaggattcttacagaaaa

SEQ ID NO: 248 - DNA, 23 bp, Artificial Sequence
  source: /note=“Description of Artificial Sequence Synthetic primer”
gcacaaatgctgaaagctgtacc

SEQ ID NO: 249 - DNA, 23 bp, Artificial Sequence
  source: /note=“Description of Artificial Sequence Synthetic primer”
caagtgtgatttgccaacaagga

SEQ ID NO: 250 - DNA, 23 bp, Artificial Sequence
  source: /note=“Description of Artificial Sequence Synthetic primer”
gcactgggactttggtaattcac

SEQ ID NO: 251 - DNA, 25 bp, Artificial Sequence
  source: /note=“Description of Artificial Sequence Synthetic primer”
catctcttggaaactcccatcttga

SEQ ID NO: 252 - DNA, 26 bp, Artificial Sequence
  source: /note=“Description of Artificial Sequence Synthetic primer”
cagtgaaaaacaagctctcatgtctg

SEQ ID NO: 253 - DNA, 24 bp, Artificial Sequence
  source: /note=“Description of Artificial Sequence Synthetic primer”
ccacatgtgtccagtgaaaatcct

SEQ ID NO: 254 - DNA, 21 bp, Artificial Sequence
  source: /note=“Description of Artificial Sequence Synthetic primer”
tgtccccataggccccattta

SEQ ID NO: 255 - DNA, 22 bp, Artificial Sequence
  source: /note=“Description of Artificial Sequence Synthetic primer”
tgctttcatcagcagggttcaa

SEQ ID NO: 256 - DNA, 20 bp, Artificial Sequence
  source: /note=“Description of Artificial Sequence Synthetic primer”
cagtgtgtccaccgtgatct

SEQ ID NO: 257 - DNA, 21 bp, Artificial Sequence
  source: /note=“Description of Artificial Sequence Synthetic primer”
agtgaaggaggatgagcctga

SEQ ID NO: 258 - DNA, 32 bp, Artificial Sequence
  source: /note=“Description of Artificial Sequence Synthetic primer”
ccataaagcatgaactatttaaagaagcaaga

SEQ ID NO: 259 - DNA, 22 bp, Artificial Sequence
  source: /note=“Description of Artificial Sequence Synthetic primer”
ggttgaaaaagccgaaggtcac

SEQ ID NO: 260 - DNA, 28 bp, Artificial Sequence
  source: /note=“Description of Artificial Sequence Synthetic primer”
tggaatgccagaactacaatcttttgat

SEQ ID NO: 261 - DNA, 28 bp, Artificial Sequence
  source: /note=“Description of Artificial Sequence Synthetic primer”
gtggaagatccaatccatttttgttgtc

SEQ ID NO: 262 - DNA, 27 bp, Artificial Sequence
  source: /note=“Description of Artificial Sequence Synthetic primer”
tggatcttccacacaattaaacagcat

SEQ ID NO: 263 - DNA, 24 bp, Artificial Sequence
  source: /note=“Description of Artificial Sequence Synthetic primer”
tgctgttcatggattgtgcaattc

SEQ ID NO: 264 - DNA, 21 bp, Artificial Sequence
  source: /note=“Description of Artificial Sequence Synthetic primer”
gacgcatttccacagctacac

SEQ ID NO: 265 - DNA, 28 bp, Artificial Sequence
  source: /note=“Description of Artificial Sequence Synthetic primer”
agcatcagcatttgactttaccttatca

SEQ ID NO: 266 - DNA, 28 bp, Artificial Sequence
  source: /note=“Description of Artificial Sequence Synthetic primer”
cataggtggaatgaatggctgaattatg

SEQ ID NO: 267 - DNA, 28 bp, Artificial Sequence
  source: /note=“Description of Artificial Sequence Synthetic primer”
tcaatcagcggtataatcaggagttttt

SEQ ID NO: 268 - DNA, 24 bp, Artificial Sequence
  source: /note=“Description of Artificial Sequence Synthetic primer”
gctttgaatctttggccagtacct

SEQ ID NO: 269 - DNA, 27 bp, Artificial Sequence
  source: /note=“Description of Artificial Sequence Synthetic primer”
cataagagagaaggtttgactgccata

SEQ ID NO: 270 - DNA, 29 bp, Artificial Sequence
  source: /note=“Description of Artificial Sequence Synthetic primer”
cagagtaacagactagctagagacaatga

SEQ ID NO: 271 - DNA, 25 bp, Artificial Sequence
  source: /note=“Description of Artificial Sequence Synthetic primer”
gcacttacctgtgactccatagaaa

SEQ ID NO: 272 - DNA, 23 bp, Artificial Sequence
  source: /note=“Description of Artificial Sequence Synthetic primer”
gatgcagccattgacctgtttac

SEQ ID NO: 273 - DNA, 26 bp, Artificial Sequence
  source: /note=“Description of Artificial Sequence Synthetic primer”
agaaaaccattacttgtccatcgtct

SEQ ID NO: 274 - DNA, 24 bp, Artificial Sequence
  source: /note=“Description of Artificial Sequence Synthetic primer”
gccatctctctcctcctttttctt

SEQ ID NO: 275 - DNA, 22 bp, Artificial Sequence
  source: /note=“Description of Artificial Sequence Synthetic primer”
gccgcagaaatggatacaggtc

SEQ ID NO: 276 - DNA, 28 bp, Artificial Sequence
  source: /note=“Description of Artificial Sequence Synthetic primer”
tgttaatggtggctttttgtttgtttgt

SEQ ID NO: 277 - DNA, 28 bp, Artificial Sequence
  source: /note=“Description of Artificial Sequence Synthetic primer”
tctacctcactctaacaagcagataact

SEQ ID NO: 278 - DNA, 22 bp, Artificial Sequence
  source: /note=“Description of Artificial Sequence Synthetic primer”
ccataacccaccacagctagaa

SEQ ID NO: 279 - DNA, 26 bp, Artificial Sequence
  source: /note=“Description of Artificial Sequence Synthetic primer”
tgccccgatgtaataaatatgcacat

SEQ ID NO: 280 - DNA, 22 bp, Artificial Sequence
  source: /note=“Description of Artificial Sequence Synthetic primer”
ggctacgacccagttaccatag

SEQ ID NO: 281 - DNA, 26 bp, Artificial Sequence
  source: /note=“Description of Artificial Sequence Synthetic primer”
tgccactggtctataatccagatgat

SEQ ID NO: 282 - DNA, 28 bp, Artificial Sequence
  source: /note=“Description of Artificial Sequence Synthetic primer”
tgagatcaagattgcagatacagaatcc

SEQ ID NO: 283 - DNA, 20 bp, Artificial Sequence
  source: /note=“Description of Artificial Sequence Synthetic primer”
acctttagctggcagaccac

SEQ ID NO: 284 - DNA, 25 bp, Artificial Sequence
  source: /note=“Description of Artificial Sequence Synthetic primer”
gcagtatagagcgtgcagataatga

SEQ ID NO: 285 - DNA, 27 bp, Artificial Sequence
  source: /note=“Description of Artificial Sequence Synthetic primer”
catcacatacatacaagtcaacaaccc

SEQ ID NO: 286 - DNA, 24 bp, Artificial Sequence
  source: /note=“Description of Artificial Sequence Synthetic primer”
gcctccctttccaatggactattt

SEQ ID NO: 287 - DNA, 24 bp, Artificial Sequence
  source: /note=“Description of Artificial Sequence Synthetic primer”
cttttaattgcccgtgatgttcca

SEQ ID NO: 288 - DNA, 25 bp, Artificial Sequence
  source: /note=“Description of Artificial Sequence Synthetic primer”
tgatgtttccttcgtaggtgttgac

SEQ ID NO: 289 - DNA, 24 bp, Artificial Sequence
  source: /note=“Description of Artificial Sequence Synthetic primer”
tggtacctgctcttcttcaatcct

SEQ ID NO: 290 - DNA, 34 bp, Artificial Sequence
  source: /note=“Description of Artificial Sequence Synthetic primer”
acttttttctattctttcctttgtagtgtccata

SEQ ID NO: 291 - DNA, 24 bp, Artificial Sequence
  source: /note=“Description of Artificial Sequence Synthetic primer”
cctttccaatttgctgaagagtgc

SEQ ID NO: 292 - DNA, 26 bp, Artificial Sequence
  source: /note=“Description of Artificial Sequence Synthetic primer”
tcttcctcagacattcaaacgtgttt

SEQ ID NO: 293 - DNA, 21 bp, Artificial Sequence
  source: /note=“Description of Artificial Sequence Synthetic primer”
acctaccctggtggaagcata

SEQ ID NO: 294 - DNA, 26 bp, Artificial Sequence
  source: /note=“Description of Artificial Sequence Synthetic primer”
gcattggtgctaaaagtttcttggat

SEQ ID NO: 295 - DNA, 23 bp, Artificial Sequence
  source: /note=“Description of Artificial Sequence Synthetic primer”
aagcagagaatgagggaggagta

SEQ ID NO: 296 - DNA, 28 bp, Artificial Sequence
  source: /note=“Description of Artificial Sequence Synthetic primer”
gctgagagatgtaatgacatgtaaagga

SEQ ID NO: 297 - DNA, 28 bp, Artificial Sequence
  source: /note=“Description of Artificial Sequence Synthetic primer”
ccatgtgcaatacctgtctatagaatca

SEQ ID NO: 298 - DNA, 28 bp, Artificial Sequence
  source: /note=“Description of Artificial Sequence Synthetic primer”
tgagacaacagaagcattatactgcttt

SEQ ID NO: 299 - DNA, 25 bp, Artificial Sequence
  source: /note=“Description of Artificial Sequence Synthetic primer”
ctggagtgtgtggaggaattacatt

SEQ ID NO: 300 - DNA, 28 bp, Artificial Sequence
  source: /note=“Description of Artificial Sequence Synthetic primer”
agaaggcaacttgacaagagaaatgata

SEQ ID NO: 301 - DNA, 32 bp, Artificial Sequence
  source: /note=“Description of Artificial Sequence Synthetic primer”
caataatttgttagccatatgcacatgaatga

SEQ ID NO: 302 - DNA, 30 bp, Artificial Sequence
  source: /note=“Description of Artificial Sequence Synthetic primer”
ctgggaaaattatgcttactaatgtggttt

SEQ ID NO: 303 - DNA, 25 bp, Artificial Sequence
  source: /note=“Description of Artificial Sequence Synthetic primer”
acaagcagattcaaggtgatcagtt

SEQ ID NO: 304 - DNA, 37 bp, Artificial Sequence
  source: /note=“Description of Artificial Sequence Synthetic primer”
agtaaaaatgactaatttttcttattcccacagtgta

SEQ ID NO: 305 - DNA, 25 bp, Artificial Sequence
  source: /note=“Description of Artificial Sequence Synthetic primer”
tgcctgtctctcatgagttcatact

SEQ ID NO: 306 - DNA, 33 bp, Artificial Sequence
  source: /note=“Description of Artificial Sequence Synthetic primer”
aacaaaaccatgtaataaaattctgactacttt

SEQ ID NO: 307 - DNA, 25 bp, Artificial Sequence
  source: /note=“Description of Artificial Sequence Synthetic primer”
gaggaagatccttgtatgctgttac

SEQ ID NO: 308 - DNA, 22 bp, Artificial Sequence
  source: /note=“Description of Artificial Sequence Synthetic primer”
gggattaaagctggctatggca

SEQ ID NO: 309 - DNA, 21 bp, Artificial Sequence
  source: /note=“Description of Artificial Sequence Synthetic primer”
ccttgttgggacctcagatgt

SEQ ID NO: 310 - DNA, 19 bp, Artificial Sequence
  source: /note=“Description of Artificial Sequence Synthetic primer”
agcatacgcagcctgtacc

SEQ ID NO: 311 - DNA, 21 bp, Artificial Sequence
  source: /note=“Description of Artificial Sequence Synthetic primer”
gtggtagcagtggatgcagaa

SEQ ID NO: 312 - DNA, 20 bp, Artificial Sequence
  source: /note=“Description of Artificial Sequence Synthetic primer”
gcttccaggagcgatcgttt

SEQ ID NO: 313 - DNA, 23 bp, Artificial Sequence
  source: /note=“Description of Artificial Sequence Synthetic primer”
aggccccatacaatttgatgaca

SEQ ID NO: 314 - DNA, 23 bp, Artificial Sequence
  source: /note=“Description of Artificial Sequence Synthetic primer”
ctggttactgaaagctcagggat

SEQ ID NO: 315 - DNA, 26 bp, Artificial Sequence
  source: /note=“Description of Artificial Sequence Synthetic primer”
actttgcgtggtgtagatatgatcaa

SEQ ID NO: 316 - DNA, 22 bp, Artificial Sequence
  source: /note=“Description of Artificial Sequence Synthetic primer”
ctcatgtgatctatgcccgtct

SEQ ID NO: 317 - DNA, 28 bp, Artificial Sequence
  source: /note=“Description of Artificial Sequence Synthetic primer”
agtctacttaccaattccaggtgataca

SEQ ID NO: 318 - DNA, 26 bp, Artificial Sequence
  source: /note=“Description of Artificial Sequence Synthetic primer”
tgctacttctgaattgaaatggttca

SEQ ID NO: 319 - DNA, 27 bp, Artificial Sequence
  source: /note=“Description of Artificial Sequence Synthetic primer”
gattacctaccattactctgcagtgtt

SEQ ID NO: 320 - DNA, 26 bp, Artificial Sequence
  source: /note=“Description of Artificial Sequence Synthetic primer”
atggtgaaggatgaatatgtgcatga

SEQ ID NO: 321 - DNA, 24 bp, Artificial Sequence
  source: /note=“Description of Artificial Sequence Synthetic primer”
gctggtagcattagactcagatgg

SEQ ID NO: 322 - DNA, 24 bp, Artificial Sequence
  source: /note=“Description of Artificial Sequence Synthetic primer”
gtgaaggactgttgcagatagcat

SEQ ID NO: 323 - DNA, 22 bp, Artificial Sequence
  source: /note=“Description of Artificial Sequence Synthetic primer”
aaggcccacatgggttaatttg

SEQ ID NO: 324 - DNA, 25 bp, Artificial Sequence
  source: /note=“Description of Artificial Sequence Synthetic primer”
tttctttagggcctgttcacaatga

SEQ ID NO: 325 - DNA, 26 bp, Artificial Sequence
  source: /note=“Description of Artificial Sequence Synthetic primer”
ctgagaagtgaccccataattccatt

SEQ ID NO: 326 - DNA, 23 bp, Artificial Sequence
  source: /note=“Description of Artificial Sequence Synthetic primer”
gctcctgagtattggtgttccat

SEQ ID NO: 327 - DNA, 22 bp, Artificial Sequence
  source: /note=“Description of Artificial Sequence Synthetic primer”
cctgtggacattggagagttga

SEQ ID NO: 328 - DNA, 35 bp, Artificial Sequence
  source: /note=“Description of Artificial Sequence Synthetic primer”
tgtaatttcttttttcttcctaaggttgcacatag

SEQ ID NO: 329 - DNA, 26 bp, Artificial Sequence
  source: /note=“Description of Artificial Sequence Synthetic primer”
acttgggtagatcttatgaacagcat

SEQ ID NO: 330 - DNA, 23 bp, Artificial Sequence
  source: /note=“Description of Artificial Sequence Synthetic primer”
aggtctttgatttgcgtcagtgt

SEQ ID NO: 331 - DNA, 22 bp, Artificial Sequence
  source: /note=“Description of Artificial Sequence Synthetic primer”
gctggagctattccacctactg

SEQ ID NO: 332 - DNA, 22 bp, Artificial Sequence
  source: /note=“Description of Artificial Sequence Synthetic primer”
gctgctggaattggtgttgatg

SEQ ID NO: 333 - DNA, 23 bp, Artificial Sequence
  source: /note=“Description of Artificial Sequence Synthetic primer”
agtacttcgtctaggagctggag

SEQ ID NO: 334 - DNA, 27 bp, Artificial Sequence
  source: /note=“Description of Artificial Sequence Synthetic primer”
cttgctttactcataggtgggaaacta

SEQ ID NO: 335 - DNA, 23 bp, Artificial Sequence
  source: /note=“Description of Artificial Sequence Synthetic primer”
acgcacccttagtgttaggtttt

SEQ ID NO: 336 - DNA, 20 bp, Artificial Sequence
  source: /note=“Description of Artificial Sequence Synthetic primer”
gctcccaccacttagatgcc

SEQ ID NO: 337 - DNA, 24 bp, Artificial Sequence
  source: /note=“Description of Artificial Sequence Synthetic primer”
aactgaaacgtgctggagaactaa

SEQ ID NO: 338 - DNA, 24 bp, Artificial Sequence
  source: /note=“Description of Artificial Sequence Synthetic primer”
ctgactgttgcttccatttcactt

SEQ ID NO: 339 - DNA, 24 bp, Artificial Sequence
  source: /note=“Description of Artificial Sequence Synthetic primer”
gactgccttgtaccattcatgttc

SEQ ID NO: 340 - DNA, 20 bp, Artificial Sequence
  source: /note=“Description of Artificial Sequence Synthetic primer”
cacttggctgccctgtagag

SEQ ID NO: 341 - DNA, 22 bp, Artificial Sequence
  source: /note=“Description of Artificial Sequence Synthetic primer”
ccaatcttctgagatgctccgt

SEQ ID NO: 342 - DNA, 24 bp, Artificial Sequence
  source: /note=“Description of Artificial Sequence Synthetic primer”
ccagaatgaggtgcagaacatcaa

SEQ ID NO: 343 - DNA, 21 bp, Artificial Sequence
  source: /note=“Description of Artificial Sequence Synthetic primer”
cgatgtagctgtgcatgtcct

SEQ ID NO: 344 - DNA, 23 bp, Artificial Sequence
  source: /note=“Description of Artificial Sequence Synthetic primer”
caggtagagggagtacagagtga

SEQ ID NO: 345 - DNA, 21 bp, Artificial Sequence
  source: /note=“Description of Artificial Sequence Synthetic primer”
ggcataggtgaggaccacaaa

SEQ ID NO: 346 - DNA, 25 bp, Artificial Sequence
  source: /note=“Description of Artificial Sequence Synthetic primer”
ggactctgtgagtgggatttgtttt

SEQ ID NO: 347 - DNA, 21 bp, Artificial Sequence
  source: /note=“Description of Artificial Sequence Synthetic primer”
gtcttcactcacctcggatga

SEQ ID NO: 348 - DNA, 24 bp, Artificial Sequence
  source: /note=“Description of Artificial Sequence Synthetic primer”
catccctgactgtgagatcaagaa

SEQ ID NO: 349 - DNA, 20 bp, Artificial Sequence
  source: /note=“Description of Artificial Sequence Synthetic primer”
caggtacgcctccagatgag

SEQ ID NO: 350 - DNA, 21 bp, Artificial Sequence
  source: /note=“Description of Artificial Sequence Synthetic primer”
ggcttggcctttgacctcaat

SEQ ID NO: 351 - DNA, 25 bp, Artificial Sequence
  source: /note=“Description of Artificial Sequence Synthetic primer”
tcctccagaagcttgaactctcata

SEQ ID NO: 352 - DNA, 21 bp, Artificial Sequence
  source: /note=“Description of Artificial Sequence Synthetic primer”
aacatcaccacgggtctgtac

SEQ ID NO: 353 - DNA, 21 bp, Artificial Sequence
  source: /note=“Description of Artificial Sequence Synthetic primer”
gatgaggctcccacctttcag

SEQ ID NO: 354 - DNA, 22 bp, Artificial Sequence
  source: /note=“Description of Artificial Sequence Synthetic primer”
gaagaaacatcctccggctgaa

SEQ ID NO: 355 - DNA, 22 bp, Artificial Sequence
  source: /note=“Description of Artificial Sequence Synthetic primer”
accgtgaagtcctgagtgtaga

SEQ ID NO: 356 - DNA, 24 bp, Artificial Sequence
  source: /note=“Description of Artificial Sequence Synthetic primer”
tccactcacagtttccataggtct

SEQ ID NO: 357 - DNA, 23 bp, Artificial Sequence
  source: /note=“Description of Artificial Sequence Synthetic primer”
gttggaagtgtctcatgctggat

SEQ ID NO: 358 - DNA, 21 bp, Artificial Sequence
  source: /note=“Description of Artificial Sequence Synthetic primer”
ggctgtcccagaatgcaagaa

SEQ ID NO: 359 - DNA, 21 bp, Artificial Sequence
  source: /note=“Description of Artificial Sequence Synthetic primer”
gatgaagctcccagaatgcca

SEQ ID NO: 360 - DNA, 21 bp, Artificial Sequence
  source: /note=“Description of Artificial Sequence Synthetic primer”
ccagttgcaaaccagacctca

SEQ ID NO: 361 - DNA, 22 bp, Artificial Sequence
  source: /note=“Description of Artificial Sequence Synthetic primer”
aggcctctgattcctcactgat

SEQ ID NO: 362 - DNA, 20 bp, Artificial Sequence
  source: /note=“Description of Artificial Sequence Synthetic primer”
ggctcctgacctggagtctt

SEQ ID NO: 363 - DNA, 24 bp, Artificial Sequence
  source: /note=“Description of Artificial Sequence Synthetic primer”
ctcatcttgggcctgtgttatctc

SEQ ID NO: 364 - DNA, 20 bp, Artificial Sequence
  source: /note=“Description of Artificial Sequence Synthetic primer”
cgcttcttgtcctgcttgct

SEQ ID NO: 365 - DNA, 27 bp, Artificial Sequence
  source: /note=“Description of Artificial Sequence Synthetic primer”
ttctcttttcctatcctgagtagtggt

SEQ ID NO: 366 - DNA, 22 bp, Artificial Sequence
  source: /note=“Description of Artificial Sequence Synthetic primer”
ctcccaggtcatcttctgcaat

SEQ ID NO: 367 - DNA, 21 bp, Artificial Sequence
  source: /note=“Description of Artificial Sequence Synthetic primer”
gtacctcggtagctgtggatg

SEQ ID NO: 368 - DNA, 23 bp, Artificial Sequence
  source: /note=“Description of Artificial Sequence Synthetic primer”
gtggctctttaacaacctttgct

SEQ ID NO: 369 - DNA, 22 bp, Artificial Sequence
  source: /note=“Description of Artificial Sequence Synthetic primer”
gtcagtacctggcagtgtgata

SEQ ID NO: 370 - DNA, 21 bp, Artificial Sequence
  source: /note=“Description of Artificial Sequence Synthetic primer”
ggcaaagcctcttgttcgttc

SEQ ID NO: 371 - DNA, 24 bp, Artificial Sequence
  source: /note=“Description of Artificial Sequence Synthetic primer”
tgacgatgtccagtctcctgtaat